(19) United States
(12) Patent Application Publication
(10) Pub. No.: US 2025/0074942 A1
(43) Pub. Date: Mar. 6, 2025

(54) COMPOSITIONS AND METHODS FOR COVALENT PEPTIDE-BASED MODULATORS OF HLA-E

(71) Applicants: Calico Life Sciences LLC, South San Francisco, CA (US); Massachusetts Institute of Technology, Cambridge, MA (US)

(72) Inventors: Magdalena Preciado Lopez, South San Francisco, CA (US); Dan L. Eaton, South San Francisco, CA (US); Clint Remarcik, South San Francisco, CA (US); John Wang, South San Francisco, CA (US); Nathalie M. Grob, Cambridge, MA (US); Simon L. Rössler, Cambridge, MA (US); Anthony J. Quartararo, Boston, MA (US); Jason Tao, Cambridge, MA (US); Andrei Loas, Boston, MA (US); Stephen L. Buchwald, Cambridge, MA (US); Bradley L. Pentelute, Revere, MA (US); Ying Kit Jeffrey Wong, Cambridge, MA (US)

(21) Appl. No.: 18/952,930

(22) Filed: Nov. 19, 2024

Related U.S. Application Data
(63) Continuation of application No. PCT/US2023/022979, filed on May 19, 2023.
(60) Provisional application No. 63/344,512, filed on May 20, 2022.

(51) Int. Cl. C07K 7/06; A61K 47/64

(52) U.S. Cl. CPC C07K 7/06; A61K 47/645

(57) ABSTRACT

This disclosure relates to synthetic peptides, peptidomimetics, and complexes of synthetic peptides and peptidomimetics with HLA-E, methods of making such peptides, peptidomimetics, and complexes, and methods of using such peptides, peptidomimetics and complexes for blocking, inhibiting, or preventing the interaction of HLA-E with CD94/NKG2A or activation of CD94/NKG2A by HLA-E. The synthetic peptides, peptidomimetics, and complexes of synthetic peptides and peptidomimetics with HLA-E can further comprise warheads to introduce covalent linkages between the synthetic peptides and peptidomimetics with HLA-E.

### CROSS-REFERENCE TO RELATED APPLICATION

This application is a continuation of International Application No. PCT/US2023/022979, filed May 19, 2023, which claims the benefit of and priority to U.S. Provisional Patent Application No. 63/344,512, filed on May 20, 2022; the disclosure of each of which is hereby incorporated by reference in their entireties for all purposes.

### SEQUENCE LISTING

This application contains a Sequence Listing that has been submitted electronically in XML format and is hereby incorporated by reference in its entirety. The XML file, created on Oct. 13, 2023, is named CLS-027WO_SL.xml, and is 578,858 bytes in size.

### BACKGROUND

Natural Killer cells (NK cells) and T cells play an important role in the innate and adaptive immune response and in the prevention of cancer. These cells provide an efficient immunosurveillance mechanism by which undesired cells such as tumor cells or virally-infected cells can be eliminated. NK cell and T cell activity is regulated by a complex mechanism that involves both activating and inhibitory signals. The inhibitory NK cell receptor dimer CD94/NKG2A C-type lectin receptor complex has recently been identified as an immune checkpoint in the tumor microenvironment and is expressed on NK cells as well as some T cell subsets. Interactions of the CD94/NKG2A with its ligand a peptide loaded histocompatibility leucocyte antigen E (HLA-E) prevent NK cells or T cells from killing healthy cells. The expression of HLA-E has also been associated with different types of cancer as a mechanism to evade attacks by NK cells or T cells. High levels of HLA-E expression are reported in several cancer types, including gynecologic cancers (up to 90% of tumor samples) and up to 50% in breast cancer, non-small cell lung carcinoma (NSCLC), liver, pancreas, kidney, melanoma, prostate, head and neck, stomach, rectal, and colorectal cancer. Blocking of the CD94/NKG2A receptor on NK and T cells has been shown to increase cytotoxic activity of NK and T cells. Recently, the antibody Monalizumab, a humanized anti-NKG2A antibody, has shown to result in enhanced NK cell activity against various tumor cells and rescued CD8+ T cell function in combination with PD-x axis blockade.

Despite the efforts that have been made to date to block the inhibitory activity of CD94/NKG2A, there is an ongoing need for new and effective treatment modalities for inhibiting CD94/NKG2A function in cancer.

### SUMMARY

The present disclosure is directed, at least in part, to synthetic peptides, peptidomimetics, compositions, and methods for the modulation of HLA-E-CD94/NKG2A interaction (e.g., activation of CD94/NKG2A signaling). In some embodiments, disclosed herein are synthetic peptides comprising an amino acid sequence X-Met-X-X-Z-Ala-X-U-Leu (SEQ ID NO: 3), wherein X is 4Af, hAr, Ala, Dff, Ser, Msn, Gln, Cha, or Arg; Z is Ala, Cha, Tha, or Mff; and U is Arg, Msn, or hAR. In some embodiments, the amino acid sequence of the synthetic peptide is (a) NH2-4Af-Met-Dff-Ser-Ala-Ala-Cha-Arg-Leu-OH (SEQ ID NO: 5); (b) NH2-hAr-Met-Msn-Dff-Cha-Ala-Arg-Msn-Leu-OH (SEQ ID NO: 6); (c) NH2-Ala-Met-hAr-Dff-Tha-Ala-Cha-Arg-Leu-OH (SEQ ID NO: 7); (d) NH2-4Af-Met-Ala-Ser-Ala-Ala-Cha-Arg-Leu-OH (SEQ ID NO: 8); or (e) NH2-hAR-Met-hAR-Gln-Mff-Ala-Cha-hAR-Leu-OH (SEQ ID NO: 9).

In another aspect, the disclosure is directed to a synthetic peptide comprising an amino acid sequence hAr-X-hAr-Gln-Mff-A-Cha-hAr-Z (SEQ ID NO: 20) wherein X is NIe or Mox; and Z is Leu, Aoa, or Cha. In some embodiments, the amino acid sequence of the synthetic peptide is (a) NH2-hAr-Nle-hAr-Gln-Dff-Ala-Cha-hAr-Leu-OH (SEQ ID NO: 21); (b) NH2-hAr-Mox-hAr-Gln-Mff-Ala-Cha-hAr-Leu-OH (SEQ ID NO: 22); (c) NH2-hAr-Met-hAr-Gln-Mff-Ala-Cha-hAr-Nle-OH (SEQ ID NO: 23); (d) NH2-hAr-Nle-hAr-Gln-Mff-Ala-Cha-hAr-Aoa-OH (SEQ ID NO: 24); or (e) NH2-hAr-Nle-hAr-Gin-Mff-Ala-Cha-hAr-Cha-OH (SEQ ID NO: 25).

In another aspect, the disclosure is directed to a synthetic peptide comprising an amino acid sequence VMAPRT(L/V)(V/L/I/F)L wherein one or more amino acids are substituted with a Cys, Lys, Tyr, His, Ser, or Thr. In some embodiments, the amino acid sequence of the synthetic peptide is VMAPRTLFL In some embodiments, the synthetic peptide comprises a substitution of the V residue at a position 1; a substitution of the M residue at a position 2; a substitution of the A residue at a position 3; a substitution of the P residue at a position 4; a substitution of the R residue at a position 5; a substitution of the T residue at a position 6; a substitution of the L residue at a position 7; a substitution of the F residue at a position 8; a substitution of the L residue at a position 9; or a combination of any of the foregoing substitutions. In some embodiments, the substitution is at position 1 and the amino acid is substituted for a 4Af, hAr, Ala, Dff, Ser, Msn. Gln, Cha, or Arg. In some embodiments, the substitution is at position 3 and the amino acid is substituted for a 4Af, hAr, Ala, Dff, Ser, Msn, Gln, Cha, or Arg. In some embodiments, the substitution is at position 4 and the amino acid is substituted for a 4Af, hAr, Ala, Dff, Ser, Msn, Gln, Cha, or Arg. In some embodiments, the substitution is at position 5 and the amino acid is substituted for an Ala, Cha, Tha, or Mff, in some embodiments, the substitution is at position 7 and the amino acid is substituted for a 4Af, hAr, Ala, Dff, Ser, Msn, Gln, Cha, or Arg. In some embodiments, the substitution is at position 8 and the amino acid is substituted for a Arg, Msn, or hAR. In some embodiments, the substitution is at position 3 and the amino acid is substituted for a Cys, Lys, Tyr, His, Ser, or Thr. In some embodiments, the substitution is at position 8 and the amino acid is substituted for a Cys, Lys, Tyr, His, Ser, or Thr.

In some embodiments, one or more amino acids of the synthetic peptide is substituted with a Cys, Lys. Tyr, His, Ser, or Thr. In some embodiments, the substituted amino acid is at position 3 or position 8. In some embodiments, the substituted amino acid is at position 8 and is substituted with a Cys. In some embodiments, the substituted amino acid is at position 3 and is substituted with a Cys.

In some embodiments, one or more Cys, Lys, Tyr, His, Ser, or Thr of the synthetic peptide is arylated. In some embodiments, the one or more Cys, Lys, Tyr, His, Ser, or Thr is conjugated to a warhead. In some embodiments, the warhead is

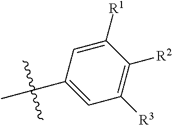

wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In some embodiments, the warhead is

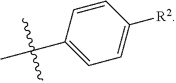

In some embodiments, the warhead is

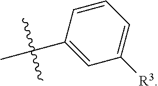

In some embodiments, the warhead is

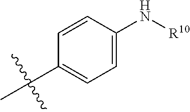

wherein R¹⁰ is hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂—N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In some embodiments, the warhead is

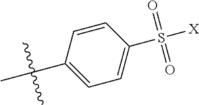

wherein X is a halogen.

In some embodiments, the warhead is

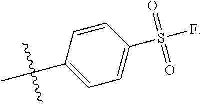 [SMILES: *c1ccc(S(=O)(=O)F)cc1]

In some embodiments, the warhead is selected from the group consisting of

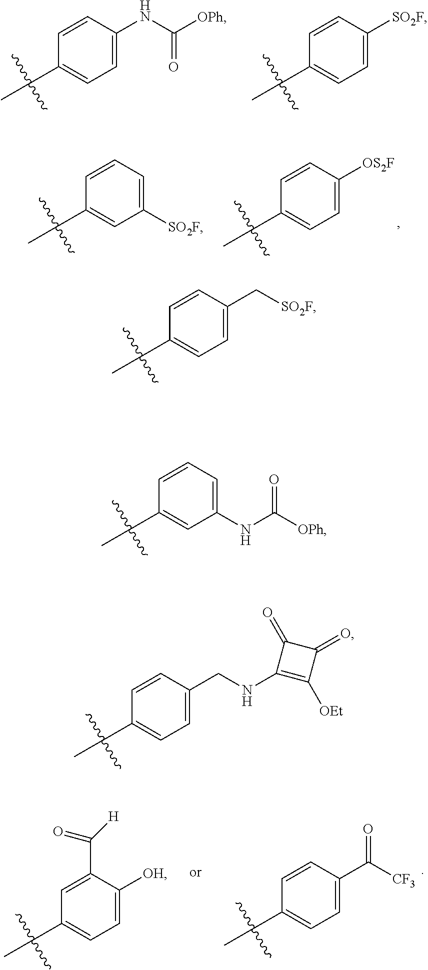

In some embodiments, the warhead is selected from the group of a sulfonyl fluoride, a phenyl carbamate, or a squareamate.

In some embodiments, the warhead is conjugated to the Cys via the Sulfur atom of the Cys.

In another aspect, the disclosure provides synthetic peptides with a warhead of the formula

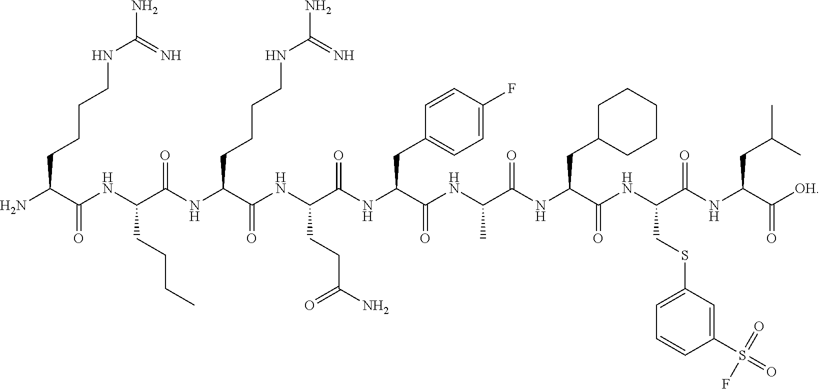 [SMILES: CCCC[C@H](NC(=O)[C@@H](N)CCCCNC(=N)N)C(=O)N[C@@H](CCCCNC(=N)N)C(=O)N[C@@H](CCC(N)=O)C(=O)N[C@@H](Cc1ccc(F)cc1)C(=O)N[C@@H](C)C(=O)N[C@@H](CC1CCCCC1)C(=O)N[C@@H](CSc1cccc(S(=O)(=O)F)c1)C(=O)N[C@@H](CC(C)C)C(=O)O]

In another aspect, the disclosure provides synthetic peptides with a warhead of the formula

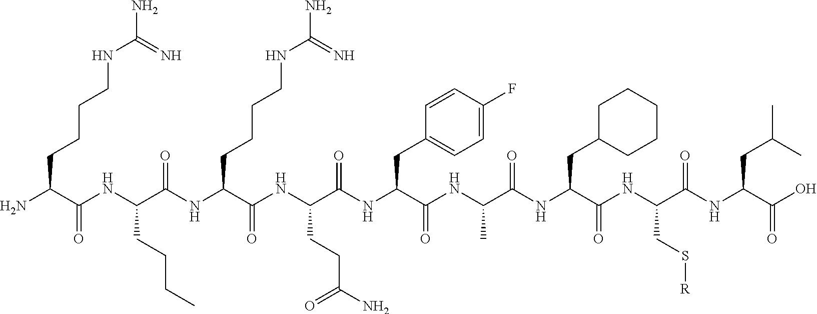

wherein the R is

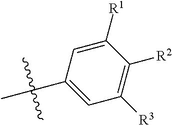

wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In another aspect, the disclosure provides synthetic peptides with a warhead of the formula

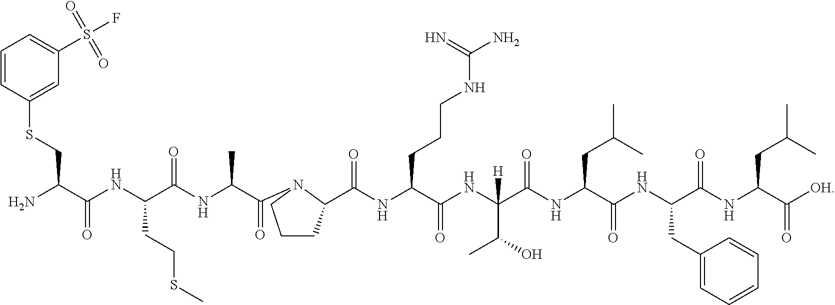 [SMILES: CSCC[C@H](NC(=O)[C@@H](N)CSc1cccc(S(=O)(=O)F)c1)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In another aspect the disclosure provide synthetic peptides with a warhead of the formula

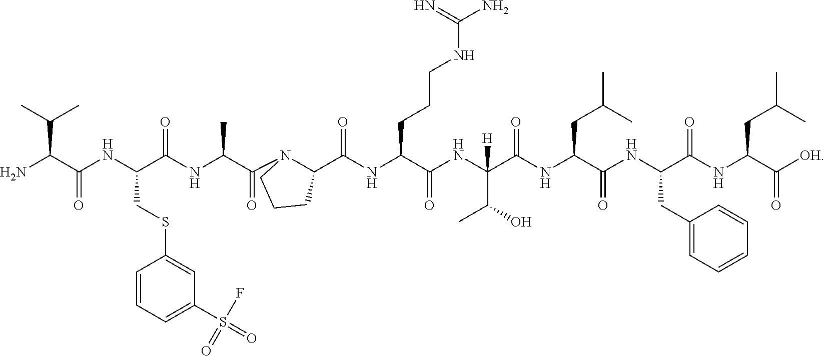 [SMILES: CC(C)C[C@H](NC(=O)[C@H](Cc1ccccc1)NC(=O)[C@H](CC(C)C)NC(=O)[C@@H](NC(=O)[C@H](CCCNC(=N)N)NC(=O)[C@@H]1CCCN1C(=O)[C@H](C)NC(=O)[C@H](CSc1cccc(S(=O)(=O)F)c1)NC(=O)[C@@H](N)C(C)C)[C@@H](C)O)C(=O)O]

In another aspect, the disclosure provides synthetic peptides with a warhead of the formula

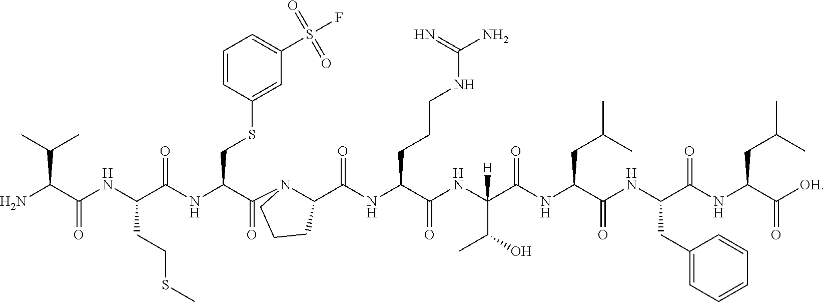 [SMILES: CSCC[C@H](NC(=O)[C@@H](N)C(C)C)C(=O)N[C@@H](CSc1cccc(S(=O)(=O)F)c1)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In another aspect, the disclosure provides synthetic peptides with a warhead of the formula

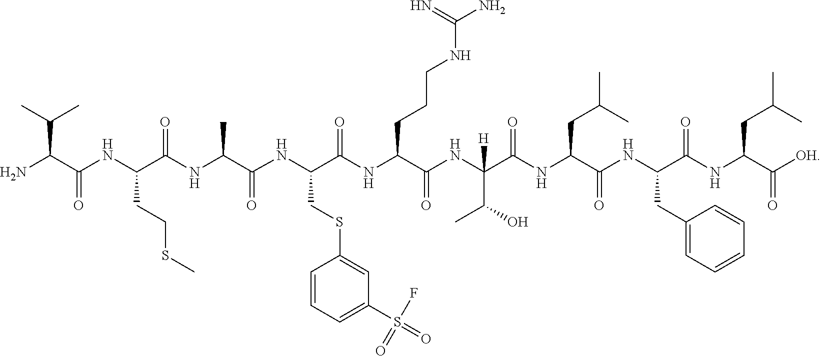 [SMILES: CSCC[C@H](NC(=O)[C@@H](N)C(C)C)C(=O)N[C@@H](C)C(=O)N[C@@H](CSc1cccc(S(=O)(=O)F)c1)C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In another aspect, the disclosure provides synthetic peptides with a warhead of the formula

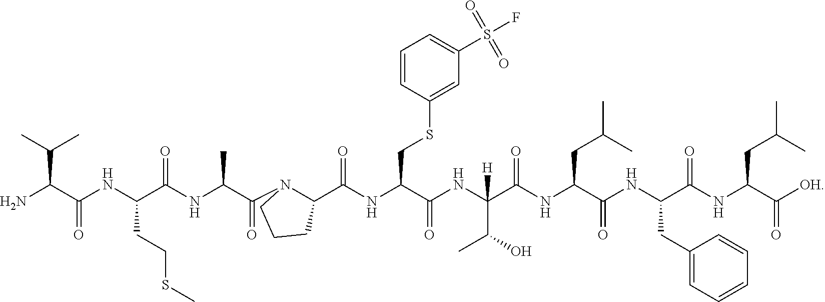 [SMILES: CSCC[C@H](NC(=O)[C@@H](N)C(C)C)C(=O)N[C@@H](C)C(=O)N1CCC[C@H]1C(=O)N[C@@H](CSc1cccc(S(=O)(=O)F)c1)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In another aspect the disclosure provides synthetic peptides with a warhead of the formula

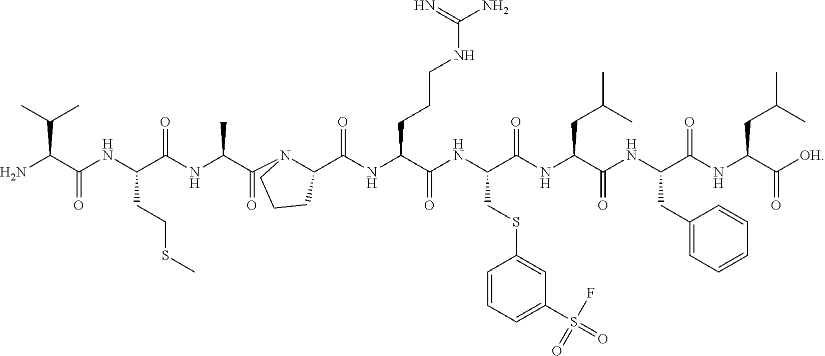 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@@H](CSc1cccc(S(=O)(=O)F)c1)C(=O)N[C@@H](CC(C)C)C(=O)N[C@@H](Cc1ccccc1)C(=O)N[C@@H](CC(C)C)C(=O)O]

In another aspect, the disclosure provides synthetic peptides with a warhead of the formula,

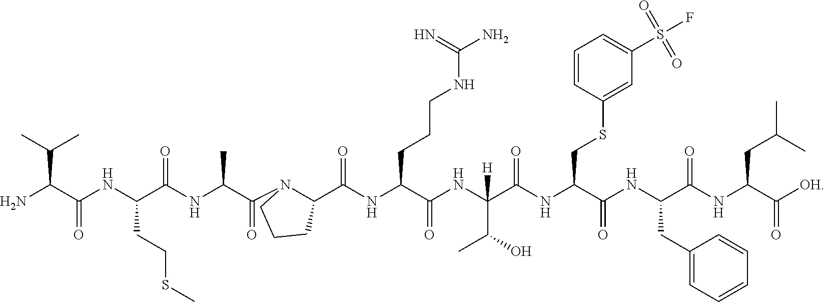 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CSc1cccc(S(=O)(=O)F)c1)C(=O)N[C@@H](Cc1ccccc1)C(=O)N[C@@H](CC(C)C)C(=O)O)[C@@H](C)O]

In another aspect the disclosure provides synthetic peptides with a warhead of the formula

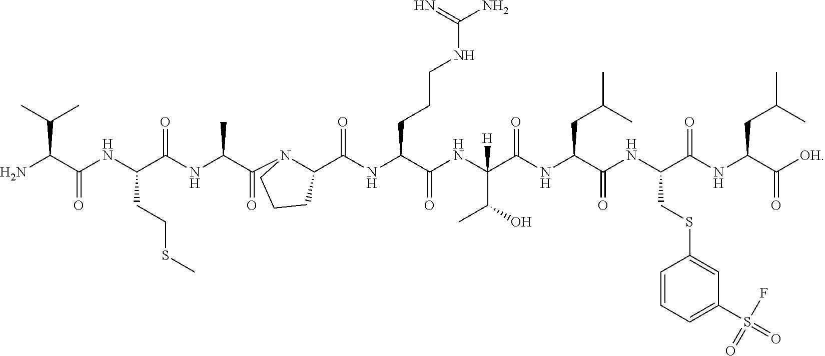 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](CSc1cccc(S(=O)(=O)F)c1)C(=O)N[C@@H](CC(C)C)C(=O)O)[C@@H](C)O]

In another aspect, the disclosure provides synthetic peptides with a warhead of the formula

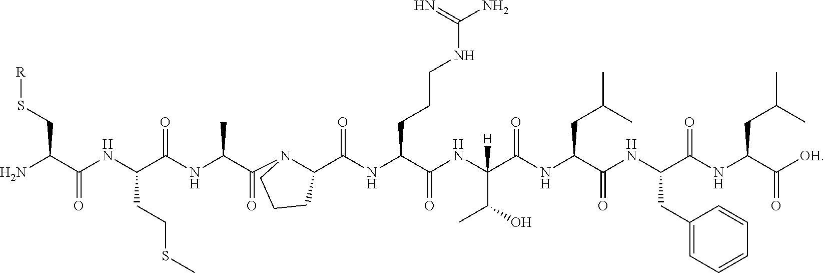

In another aspect, the disclosure provides synthetic peptides with a warhead of the formula

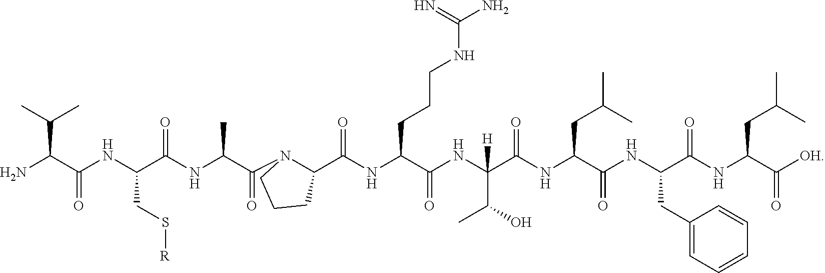

In another aspect, the disclosure provides synthetic peptides with a warhead of the formula

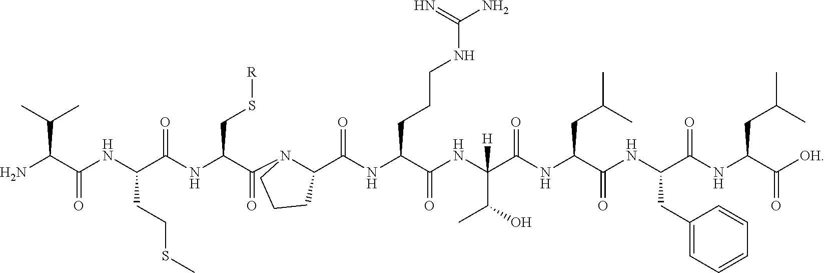

In another aspect, the disclosure provides synthetic peptides with a warhead of the formula

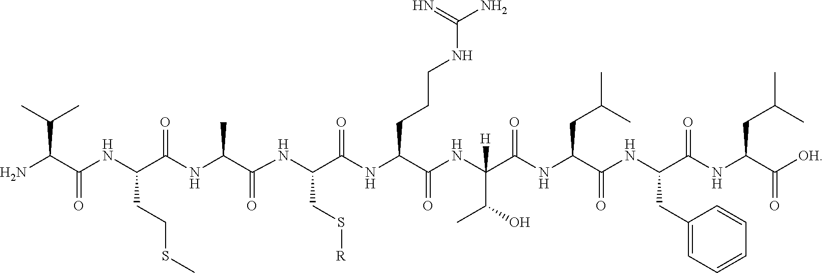

In another aspect, the disclosure provides synthetic peptides with a warhead of the formula

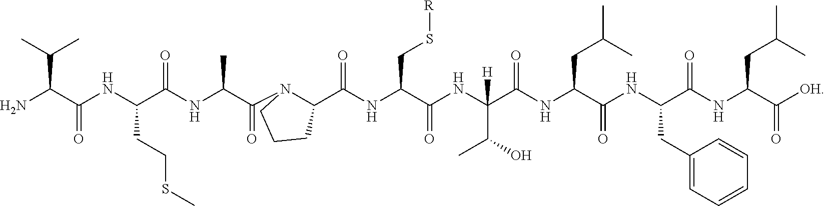

In another aspect, the disclosure provides synthetic peptides with a warhead of the formula

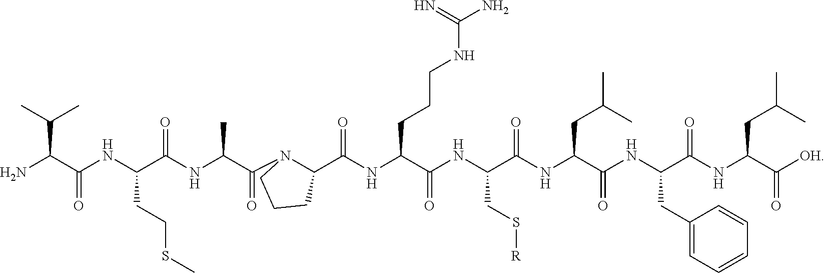

In another aspect, the disclosure provides synthetic peptides with a warhead of the formula

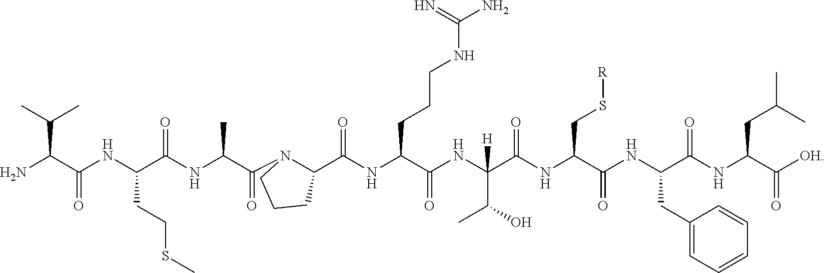

In another aspect, the disclosure provides synthetic peptides with a warhead of the formula

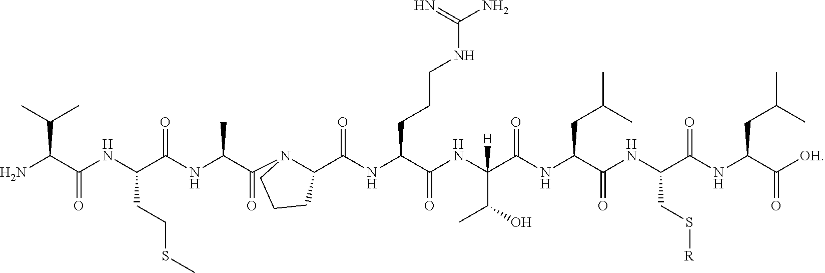

In some embodiments, R is

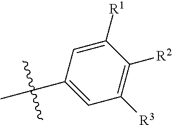

wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In another aspect, the disclosure provides synthetic peptides that are HLA-E-CD94/NKG2A complex specific inhibitor.

In some embodiments, the synthetic peptidic comprises one or more additional modifications selected from: acetylated, formylated, propanylated, hexanoylated, or myristoylated N-terminus; amidated C-terminus; substitution of one or more L-amino acid with a D-amino acid; substitution of one or more amino acid with a methyl-amino acid; or substitution of an α-amino acid with a β-amino acids.

In some embodiments, the disclosure provides a complex of the synthetic peptide with HLA-E complex. In some embodiments, the synthetic peptide and the HLA-E in the complex are covalently linked. In some embodiments, the HLA-E is human HLA-E. In some embodiments, in the synthetic peptide is covalently linked to amino acid residue Tyr-7, Lys-146, Tyr-159, or Tyr-171 of human HLA-E. In some embodiments, the synthetic peptide is covalently linked to an amino acid residue selected from the group of Tyr-7, His-9, Ser-24, Tyr-59, Arg-62, Glu-63, Ser-66, Thr-70, Gln-72, Asn-77, Thr-80, Tyr-84, Trp-97, His-99, Glu-114, Tyr-123, Trp-133, Ser-143, Lys-146, Ser-147, Glu-152, His-155, Gln-156, Tyr-159, Thr-163, Cys-164, Trp-167, and Tyr-171 of human HLA-E. In some embodiments, peptide/HLA-E complex is inhibited in binding of CD94/NKG2A or prevents activation of CD94/NKG2A.

In another aspect, the disclosure provides a synthetic peptide/HLA-E complex, wherein the synthetic peptide is covalently linked to amino acid residue Tyr-7, Tyr-171, Tyr-159, or Lys-146 of human HLA-E.

In some embodiments, the disclosure provides a pharmaceutical composition, comprising a synthetic peptide and a pharmaceutically acceptable salt or carrier.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 depicts non-canonical amino acids used in the design of a combinatorial library.

FIG. 2A-2F shows the chemical formulas for peptides B1-B5 (SEQ ID NOs: 5-9).

FIG. 3A-3F shows the chemical formulas for peptides B5.1 to B5.5, including a scrambled control peptide.

FIG. 4 illustrates the results of a peptide exchange experiment of HLA-E/VL9 with exemplary peptides B1, B2, B3, B4, B5, B5.1, B5.2, B5.3, B5.4 and B5.5.

FIG. 5A illustrates the results of a BLI experiment of binding of CD94/NKG2A to exemplary loaded HLA-E peptides (B1, B2, B3, B4, B5, and B5.1). FIG. 5B depicts the results of a BLI experiment of binding of CD94/NKG2A to exemplary loaded HLA-E peptides (B5, B5.1, B5.2, B5.3, B5.4 and B5.5). FIG. 5C depicts the results of a BLI experiment of binding of CD94/NKG2A to exemplary loaded HLA-E peptides (B5, B5.1, B5scrambled).

FIG. 6A illustrates a chemical reaction for installing an electrophilic aryl sulfonyl fluoride warhead by Pd-mediated coupling using an oxidative addition complex. FIG. 6B illustrates a reaction for cross-linking of electrophilic analogs of VL9 to HLA-E. FIG. 6C illustrates the results for exemplary cross-linking reactions with the electrophilic analogs of VL9 as measured my Mass Spectrometry (MS). FIG. 6D illustrates nucleophilic residues in a crystal structure on HLA-E situated within 6 A from the binding groove of VL9 (highlighted in yellow).

FIG. 7 illustrates an exemplary synthesis of Palladium Oxidative Addition Complex, (RuPhos)Pd(m-benzenefluorosulfonyl)Br, 1.

FIG. 8A illustrates an exemplary peptide with a warhead (B5.1_8*) that is a covalent binder selective for HLA-E and inhibits binding of CD94/NKG2A. B5.1 was equipped with an electrophilic warhead at position 8 for covalent binding. FIG. 8B illustrates the results of a BLI experiment of binding of CD94/NKG2A to exemplary loaded HLA-E peptides (B5.1_8* and VL-9_8*). FIG. 8C: shows the results of crosslinking experiments measured MS of exemplary peptide/HLA-E complexes (grey traces represent protein reference spectra prior to incubation).

### DETAILED DESCRIPTION

The present disclosure is based, in part, upon the development of synthetic peptides and peptidomimetics that bind HLA-E in a covalent or non covalent manner to form peptide-HLA-E complexes. Additionally, the peptide-HLA-E complexes can modulate or inhibit the binding of HLA-E to its cognate receptor CD94/NKG2A or prevent activation of CD94/NKG2A. The synthetic peptides and peptidomimetics can be used to modulate or abrogate HLA-E/CD94/NKG2A signaling in NK and T cells.

Various components and aspects of the disclosure are described in further detail in the subsections below.

### I. Definitions

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method provided and described herein can be combined in a variety of ways without departing from the spirit and scope of the present disclosure and invention(s) herein, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of invention(s) provided, described, and depicted herein.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

As used herein, “residue” refers to a position in a protein and its associated amino acid identity.

As used herein, “Natural Killer cell” or “NK cell” refers to a sub-population of lymphocytes that is involved in non-conventional immunity. NK cells can be identified by virtue of certain characteristics and biological properties, such as the expression of specific surface antigens including CD16, CD56, and/or CD57, the absence of the alpha/beta or gamma/delta T-cell receptor (TCR) complex on the cell surface, the ability to bind to and kill cells that fail to express “self MHC/HLA antigens by the activation of specific cytolytic enzymes, the ability to kill tumor cells or other diseased cells that express a ligand for NK-activating receptors, and the ability to release cytokines that stimulate or inhibit the immune response. Any of these characteristics and activities can be used to identify NK cells, using methods well known in the art such as fluorescence assisted cell sorting (FACS).

As used herein, “NKG2A” or “KLRC1” refers to the full length NKG2A. NKG2A (OMIM 161555, the entire disclosure of which is herein incorporated by reference) is a member of the NKG2 group of transcripts (see e.g., Houchins, et al. (1991) J. Exp.NKG2 family and their ligands). NKG2A and NKG2C form heterodimer receptors with CD94 and both target the same p/HLA-E complex, but ligation with the p/HLA-E complex induces an inhibitory signal for NKG2A and an activation signal for NKG2C. In contrast to the classical HLA class I molecules that present antigenic peptide epitopes to the TCR in complex with CD3, HLA-E presents a limited set of conserved signal peptides to NKG2A and NKG2C. These peptides bound and presented by HLA-E are derived from leader sequences of classical HLA class I molecules. The receptor dimer CD94/NKG2A found on natural killer (NK) cells recognizes these short peptides bound to human leukocyte antigen-E (HLA-E), which has an inhibitory effect on NK cells. The peptide-HLA-E complex is expressed in most human tissues as a marker of cell health and protects cells from the cytotoxic activation of NK cells. The expression of HLA-E has also been associated with different types of cancer as a mechanism to evade attacks by NK cells.

The terms “major histocompatibility complex” and “MHC” also refer to the polymorphic glycoproteins encoded by the MHC class I or class II genes, where appropriate in the context, and proteins comprising variants thereof that bind T cell epitopes (e.g., class I or class II epitopes). Such proteins are also referred to as “MHC molecule” or “MHC protein” herein. The terms “MHC class I” or “MHC I” are used interchangeably to refer to protein molecules comprising an a chain composed of three domains (α1, α2 and α3), and a second, invariant β2-microglobulin. The α3 domain is linked to the transmembrane domain, anchoring the MHC class I molecule to the cell membrane. Antigen-derived peptide epitopes, which are located in the peptide-binding groove, in the central region of the α1/α2 heterodimer. MHC Class I molecules such as HLA-A, HLA-B, HLA-C, and HLA-E are part of a process that presents short polypeptides to the immune system. These polypeptides are typically 8-11 amino acids in length and originate from proteins being expressed by the cell, which can be endogenous proteins or exogenous proteins (e.g., viral or bacterial proteins, vaccine proteins). MHC class I molecules present antigen to CD8+ cytotoxic T cells. Histocompatibility leucocyte antigen E (HLA-E), is a conserved nonclassical HLA class I molecule that binds a limited peptide repertoire. Antigens delivered endogenously to APCs are processed primarily for association with MHC class I. Antigens delivered exogenously to APCs are processed primarily for association with MHC class II. As used herein, MHC proteins (MHC Class I or Class II proteins) also includes MHC variants which contain amino acid substitutions, deletions or insertions and yet which still bind MHC peptide epitopes (MHC Class I or MHC Class II peptide epitopes). The term “MHC,” “MHC molecule,” or “MHC protein” also includes an extracellular fragment of a full-length MHC protein that retains the ability to bind the cognate epitope, for example, a soluble MHC. As used herein, the term “soluble MHC” refers to an extracellular fragment of a MHC comprising corresponding α1 and α2 domains that bind a class I T cell epitope or corresponding α1 and 31 domains that bind a class II T cell epitope, where the α1 and α2 domains or the α1 and p31 domains are derived from a naturally occurring MHC or a variant thereof. The classical MHC class I (termed “Ia”) molecules (HLA-A, HLA-B and HLA-C) are highly polymorphic and are ubiquitously expressed on most somatic cells. In contrast, non classical MHC class I (termed “Ib”) molecules (HLA-E, HLA-F and HLA-G) are broadly defined by a limited polymorphism and a restricted pattern of cellular expression.

The term “HLA-E” refers wild type, full length HLA-E. Among class Ib molecules, HLA-E is characterized by a low polymorphism and a broad mRNA expression on different cell types. Lee et al. (1988) J Immunol. 160:4951-60. HLA-E is nonpolymorphic with only two functional alleles present in the human population: the HLA-E*01:01 and the HLA-E*01:03 variants. These two alleles only differ in a single amino acid at position 107, being arginine (01:01) or glycine (01:03). This class I molecule is a heterodimer consisting of a heavy chain and a light chain (β2-microglobulin, β2m, B2M). The heavy chain is approximately 45 kDa and its gene contains 8 exons. Cell surface expression of HLA-E requires the availability of β2-microglobulin (Ulbrecht et al. (1999) Eur J Immunol. 29:537-47) and of a set of highly conserved nonameric peptides derived from the leader sequence of various HLA class I molecules including HLA-A, —B, —C, and -G (see e.g., Braud et al. (1997) Eur J Immunol. 27: 1164-9; Ulbrecht et al. (1998) J Immunol. 160:4375-85). HLA-E binds NK cells and some T cells, binding specifically to CD94/NKG2A, CD94/NKG2B, and CD94/NKG2C, and not to the inhibitory KIR receptors. See, e.g., Braud et al. (1998) Nature 391:795-799. Surface expression of HLA-E is sufficient to protect target cells from lysis by CD94/NKG2A+ NK cell clones.

The term “MHC protein” also includes MHC proteins of non-human species of vertebrates. MHC proteins of non-human species of vertebrates play a role in the examination and healing of diseases of these species of vertebrates, for example, in veterinary medicine and in animal tests in which human diseases are examined on an animal model, for example, experimental autoimmune encephalomyelitis (EAE) in mice (Mus musculus), which is an animal model of the human disease multiple sclerosis. Non-human species of vertebrates are, for example, and more specifically mice (Mus musculus), rats (Rattus norvegicus), cows (Bos taurus), horses (Equus equus) and green monkeys (Macaca mulatta). MHC proteins of mice are, for example, referred to as H-2-proteins, wherein the MHC class I proteins are encoded by the gene loci H2K, H2L, and H2D and the MHC class II proteins are encoded by the gene loci H2I.

The term “modulation” refers to an increase or decrease in the level of a target molecule or the function of a target molecule. The term “modulator” as used herein refers to modulation of (e.g., an increase or decrease in) the level of a target molecule or the function of a target molecule.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

When a range of values is listed, it is intended to encompass each value and sub-range within the range. For example, “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

“Aryl” refers to a radical of a monocyclic or polycyclic (e.g., bicyclic or tricyclic) 4n+2 aromatic ring system (e.g., having 6, 10, or 14 p electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). An aryl group may be described as, e.g., a C6-C10-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

The use of any and all examples, or exemplary language herein, for example, “such as” or “including,” is intended merely to illustrate better the present disclosure and does not pose a limitation on the scope of any invention(s) unless claimed. No language in the specification should be construed as indicating any non-claimed element as essential to the practice of that provided by the present disclosure.

### II. Peptides and Peptidomimetics

Disclosed herein are synthetic peptides, peptidomimetics, and libraries thereof. In some embodiments, the synthetic peptides and peptidomimetics are a peptide antigen bound to and presented by the MHC class I molecule major histocompatibility complex E (HLA-E).

In some aspects of the disclosure, the peptide or peptidomimetic has the amino acid sequence of an antigen. Peptide antigens comprise, but are not limited to peptides that have the amino acid sequence VMAPRT(L/V)(V/L/I/F)L (SEQ ID NO: 1), (referred to as VL9), derived from signal peptides of the MHC class I molecules HLA-A, —B, —C, and -G. In some embodiments, the peptide or peptidomimetic is based on the ligand for the NKG2A/CD94 inhibitory receptor in mice, the nonclassical MHC molecule Qa-1b, the mouse HLA-E ortholog, which presents the peptide AMAPRTLLL (SEQ ID NO: 355), referred to as Qdm (for Qa-1 determinant modifier). This dominant peptide is derived from the leader sequences of murine classical MHC class I encoded by the H-2D and -L loci.

In some embodiments the peptide sequence comprises the amino acid sequence VMAPRTLVL (SEQ ID NO: 2). In some embodiments, the peptide is 8, 9, or 10 amino acids long. In some embodiments one or more amino acids of the VL9 sequence are substituted. In some embodiments, the substitution is a substitution of the V residue at position 1 (Val1), the M residue at position 2 (Met2), the A residue at position 3 (Ala3), P residue at position 4 (Pro4), R residue at position 5 (Arg5), the T residue at position 6 (Thr6), the L residue at position 7 (Leu7), the F residue at position 8 (Phe8), the L residue at position 9 (Leu9), or a combination of any of the foregoing substitutions. In some embodiments, the anchor residues Met2 and Leu9 are constant. In some embodiments, the residue at position 10 is a Lys. In some embodiments, the R residue at position 5 is substituted with aliphatic and aromatic monomers, and the F residue at position 8 is substituted by polar and charged residues.

In some embodiments, the peptide antigen comprises, but are not limited to peptides that have the amino acid sequence VMAPRT(L/V)(V/L/I/F)L (SEQ ID NO: 1), (referred to as VL9), derived from signal peptides of the MHC class I molecules HLA-A, —B, —C, and -G. In some embodiments, the substituted amino acid is a canonical amino acid. Canonical amino acids for use in substitutions are listed in TABLE 1. In some embodiments, the canonical substituted amino acids are an Ala, a Ser, a Gin, or an Arg. TABLE 1 Canonical amino acids used in the peptides and peptidomimetics.

TABLE 1

| Amino Acid | One Letter Code | Three Letter Code |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

In some aspects of the disclosure, the synthetic peptide or peptidomimetic comprises one or more of 43 non-canonical amino acids. In some embodiments, amino acids in VL9 are substituted with non-canonical amino acids. Non-canonical amino acids that can be used for substitution are shown in TABLE 2.

TABLE 2 Non-canonical amino acids for use in the peptides and peptidomimetics.

TABLE 2

| Non canonical Amino Acid | Three Letter Code |
|---|---|
| 4-(Boc-amino)-L-Phe-OH | 4Af |
| 3-(4′-pyridyl)-L-Ala-OH | 4Py |
| L-α-aminoadipic acid(δtBu) | Aad |
| 3-aminopropanoic acid | Abu |
| α-aminoisobutyric acid | Aib |
| N-Methyl-L-alanine | Ala(NMe) |
| (4-aminomethyl) benzoic acid | Amb |
| L-2-aminooctanoic acid | Aoa |
| β-alanine | Bal |
| (S)-2-aminohept-6-ynoic acid | Bhp |
| L-canavanine | Cav |
| β-cyclobutyl-L-Ala-OH | Cba |
| β-cyclohexyl-L-Ala-OH | Cha |
| β-cyclopropyl-L-Ala-OH | Cpa |
| (S)-2,4-diaminobutanoic acid | Dab |
| 2-(Boc)aminobutyric acid | Dab |
| (S)-2,3-diaminopropanoic acid | Dap |
| β-azido-L-alanine | Dap(N3) |
| 3,4-difluoro-L-Phe-OH | Dff |
| 2-aminoacrylic acid | Dha |
| homo-D-arginine | DhAr |
| 2,3-dimethylbutanoic acid | DMBA |
| 3,4-dimethoxy-L-Phe-OH | Dmf |
| (13C5, 15N2)-L-glutamine | Gln(13C5, 15N2) |
| Nω-(Pbf)-L-homoArg-OH | hAr |
| homo-L-cysteine | hCys |
| (S)-2-hydroxy-3-methylbutanoic acid | HMBA |
| (S)-2-hydroxy-4-methylpentanoic acid | HMPA |
| homoPhe-OH (hPh), | hPh |
| L-hydroxyproline(tBu)—OH | Hyp |
| 4-fluoro-L-phenylalanine | Mff |
| O-methyl-L-homoserine | Mox |
| L-Met sulfone-OH | Msn |
| 3-(2-naphtyl)-L-Ala-OH | Nal |
| L-norleucine | Nle |
| L-ornithine(Boc)—OH | Orn |
| 2-(2-(2-aminoethoxy)ethoxy)acetic acid | PEG2 |
| 1-amino-3,6,9,12-tetraoxapentadecan-15-oic acid | PEG4 |
| (S)-2-aminopent-4-ynoic acid | Pgl |
| 4-phenylpiperidine-4-carboxylic acid | Php |
| 3,4,5-trifluoro-L-Phe-OH | Tff |
| 3-(4-thiazolyl)-L-Ala-OH | Tha |

In some embodiments, substitutions in the VL9 peptide comprise the non-canonical amino acids 4Af, hAr, Dff, Msn, or Cha. In some embodiments the amino acids of the synthetic peptide are mixed canonical and non-canonical amino acids.

In some embodiments, the substitution is at position 1 and the amino acid is substituted for a 4Af, hAr, Ala, Dff, Ser, Msn, Gln, Cha, or Arg. In some embodiments, the substitution is at position 3 and the amino acid is substituted for a 4Af, hAr, Ala, Dff, Ser, Msn, Gln, Cha, or Arg. In some embodiments, the substitution is at position 4 and the amino acid is substituted for a 4Af, hAr, Ala, Dff, Ser, Msn, Gln, Cha, or Arg. In some embodiments, the substitution is at position 5 and the amino acid is substituted for an Ala, Cha, Tha, or Mf. In some embodiments, the substitution is at position 7 and the amino acid is substituted for a 4Af, hAr, Ala, Dff, Ser, Msn, Gln, Cha, or Arg. In some embodiments, the substitution is at position 8 and the amino acid is substituted for a Arg, Msn, or hAR. In some embodiments, the substitution is at position 3 and the amino acid is substituted for a Cys. Lys, Tyr, His, Ser, or Thr. In some embodiments, the substitution is at position 8 and the amino acid is substituted for a Cys, Lys, Tyr, His, Ser, or Thr.

In some embodiments, the amino acid sequence of the peptide or peptidomimetic comprises X-Met-X-X-Z-Ala-X-U-Leu (SEQ ID NO: 3) or X-Met-X-X-Z-Ala-X-U-Leu-Lys (SEQ ID NO: 4), wherein X=Gly, Ala, Met, Pro, Cpa, Cha, Ser, Asn, Gln, Msn, Phe, Tyr, His, Trp, 4Py, 4Af, Tha, Dff; Asp, Glu, Lys, Arg, hAr, Aad; Z=Gly, Ala, Val, Leu, Met, Pm, Cpa, Cba, Cha, Aoa, Phe, Trp, Mff, Dff, Tff, Tha, Nal, hPh, Dmf, Php, Amb; and U=Ser, Thr, Asn, Gln, Msn, Hyp, Asp, Glu, Lys, Arg, Dab, Orn, Aad, hAr.

In some embodiments, the amino acid sequence of the peptide or peptidomimetic comprises a modified side chain. Exemplary side chains that can be used to modify the peptide or peptidomimetic of the disclosure are listed in TABLE A.

TABLE A

| Abbreviation | Chemical Name |
|---|---|
| 3SF | 3-(oxo-methyl)benzenesulfonyl fluoride |
| 4SF | 4-(oxo-methyl)benzenesulfonyl fluoride |
| 3SF4OMe | 4-methoxy-3-(oxo-methyl)benzenesulfonyl fluoride |
| 5SF2OMe | 2-methoxy-5-(oxo-methyl)benzenesulfonyl fluoride |
| CUM | 7-methoxy-4-(2-oxo-2-ethyl)-2H-chromen-2-one |
| DOTA | 2,2′,2″,2″′-(1,4,7,10-Tetraazacyclododecane-1,4,7,10- |
| | tetrayl)tetraacetic acid |
| FAM | 1-(3′,6′-dihydroxy-3-oxo-3H-spiro[isobenzofuran-1,9′- |
| | xanthen]-5-yl)-4-pyrrolidine-2,5-dione |
| FITC | Fluorescein isothiocyanate |
| ibu | ibuprofen |
| GalNAc | 3-(((2S,3S,4S,5S,6R)-3,4,5-trihydroxy-6-(2- |
| | phosphonoethyl)tetrahydro-2H-pyran-2 |
| | -yl)oxy)propanoic acid |
| Hya | 2-(hydroxyamino)-1-ethan-1-one |
| mFSF | 2-fluoro-5-benzenesulfonyl fluoride |
| mMeSF | 2-methyl-5-benzenesulfonyl fluoride |
| mOSF | 3-phenyl sulfurofluoridate |
| mPC | phenyl(-3-phenyl)carbamate |
| mPySF | 5-pyridine-3-sulfonyl fluoride |
| mSF | 3-benzenesulfonyl fluoride |
| mSN | 3-phenyl sulfamate |
| NHS | 2,5-dioxopyrrolidin-1-yl 3-(4-phenyl)propanoate |
| pPC | phenyl(-4-phenyl)carbamate |
| pSF | 4-benzenesulfonyl fluoride |
| pSN | 4-phenyl sulfamate |
| SAL | 2-hydroxy-5-benzaldehyde |
| Tz-PTAD | 4-(4-((1,2,3-triazol-4-yl)methoxy)phenyl)-1,2,4- |
| | triazolidine-3,5-dione |
| TAMRA | 5-carboxytetramethylrhodamine |
| Tz-SQ | 3-(((1,2,3-triazol-4-yl)methyl)amino)-4-ethoxycyclobut-3- |
| | ene-1,2-dione |
| mACA | 1-Phenylprop-2-en-1-one |
| mSAC | N-Phenylethene-1-sulfonamide |
| mAld | m-Benzaldehyde |
| oAld | o-Benzaldehyde |

In some embodiments, amino acid sequence of the peptide or peptidomimetic is selected form the amino acid sequences in TABLE 3. In some embodiments, the substituted peptide is selected from the group of SEQ ID NOs: 5-9.

TABLE 3 exemplary amino acid sequences of peptidomimetics disclosed herein.

TABLE 3

| Amino Acid Sequence | SEQ ID NO: |
|---|---|
| NH2-4Af-Met-Dff-Ser-Ala-Ala-Cha-Arg-Leu-OH | 5 |
| NH2-hAr-Met-Msn-Dff-Cha-Ala-Arg-Msn-Leu-OH | 6 |
| NH2-Ala-Met-hAr-Dff-Tha-Ala-Cha-Arg-Leu-OH | 7 |
| NH2-4Af-Met-Ala-Ser-Ala-Ala-Cha-Arg-Leu-OH | 8 |
| NH2-hAR-Met-hAR-Gln-Mff-Ala-Cha-hAR-Leu-OH | 9 |

In some embodiments, additional amino acids in SEQ ID NOs: 3-9 are substituted. In some embodiments one or more amino acid is substituted with a Cys, Lys, Tyr, His, Ser, or Thr. In some embodiments, the substituted amino acid is at position 3 or position 8. In some embodiments, the substituted amino acid is at position 8 and is substituted with a Cys. In some embodiments, the substituted amino acid is at position 3 and is substituted with a Cys. Exemplary Cys substituted peptides and peptidomimetic sequences are listed in TABLE 4. In some embodiments, the Cys substituted peptide is selected from the group of SEQ ID NOs: 10-19.

TABLE 4 exemplary amino acid sequences of peptidomimetics disclosed herein.

TABLE 4

| Amino Acid Sequence | SEQ ID NO: |
|---|---|
| NH2-4Af-Met-Dff-Ser-Ala-Ala-Cha-Cys-Leu-OH | 10 |
| NH2-hAr-Met-Msn-Dff-Cha-Ala-Arg-Cys-Leu-OH | 11 |
| NH2-Ala-Met-hAr-Dff-Tha-Ala-Cha-Cys-Leu-OH | 12 |
| NH2-4Af-Met-Ala-Ser-Ala-Ala-Cha-Cys-Leu-OH | 13 |
| NH2-hAR-Met-hAR-Gln-Mff-Ala-Cha-Cys-Leu-OH | 14 |
| NH2-4Af-Met-Cys-Ser-Ala-Ala-Cha-Arg-Leu-OH | 15 |
| NH2-hAr-Met-Cys-Dff-Cha-Ala-Arg-Msn-Leu-OH | 16 |
| NH2-Ala-Met-Cys-Dff-Tha-Ala-Cha-Arg-Leu-OH | 17 |
| NH2-4Af-Met-Cys-Ser-Ala-Ala-Cha-Arg-Leu-OH | 18 |
| NH2-hAR-Met-Cys-Gln-Mff-Ala-Cha-hAR-Leu-OH | 19 |

In some embodiments, the amino acid sequence of the peptide or peptidomimetic comprises hAr-X-hAr-Gln-Mff-A-Cha-hAr-Z (SEQ ID NO: 20) wherein X is Nle or Mox; and Z is Leu, Aoa, or Cha.

TABLE 5 exemplary amino acid sequences of peptidomimetics disclosed herein.

TABLE 5

| Amino Acid Sequence | SEQ ID NO: |
|---|---|
| NH2-hAr-Nle-hAr-Gln-Mff-Ala-Cha-hAr-Leu-OH | 21 |
| NH2-hAr-Mox-hAr-Gln-Mff-Ala-Cha-hAr-Leu-OH | 22 |
| NH₂-hAr-Met-hAr-Gln-Mff-Ala-Cha-hAr-Nle-OH | 23 |
| NH2-hAr-Nle-hAr-Gln-Mff-Ala-Cha-hAr-Aoa-OH | 24 |
| NH2-hAr-Nle-hAr-Gln-Mff-Ala-Cha-hAr-Cha-OH | 25 |

In some embodiments, additional amino acids in SEQ ID NOs: 21-25 are substituted. In some embodiments one or more amino acid is substituted with a Cys, Lys, Tyr, His, Ser, or Thr. In some embodiments, the substituted amino acid is at position 3 or position 8. In some embodiments, the substituted amino acid is at position 8 and is substituted with a Cys. In some embodiments, the substituted amino acid is at position 3 and is substituted with a Cys. Exemplary Cys substituted peptides and peptidomimetic sequences are listed in TABLE 4. In some embodiments, the Cys substituted peptide is selected from the group of SEQ ID NOs: 26-34.

TABLE 6 lists exemplary amino acid sequences of peptidomimetics disclosed herein.

TABLE 6

| Amino Acid Sequence | SEQ ID NO: |
|---|---|
| NH2-hAr-Nle-Cys-Gln-Mff-Ala-Cha-hAr-Leu-OH | 26 |
| NH2-hAr-Nle-hAr-Gln-Mff-Ala-Cha-Cys-Leu-OH | 27 |
| NH2-hAr-Mox-Cys-Gln-Mff-Ala-Cha-hAr-Leu-OH | 28 |
| NH2-hAr-Mox-hAr-Gln-Mff-Ala-Cha-Cys-Leu-OH | 29 |
| NH₂-hAr-Met-Cys-Gln-Mff-Ala-Cha-hAr-Nle-OH | 30 |
| NH₂-hAr-Met-hAr-Gln-Mff-Ala-Cha-Cys-Nle-OH | 31 |
| NH2-hAr-Cys-hAr-Gln-Mff-Ala-Cha-hAr-Aoa-OH | 32 |
| NH2-hAr-Nle-hAr-Gln-Mff-Ala-Cha-Cys-Aoa-OH | 33 |
| NH2-hAr-Nle-Cys-Gln-Mff-Ala-Cha-hAr-Cha-OH | 34 |
| NH2-hAr-Nle-hAr-Gln-Mff-Ala-Cha-Cys-Cha-OH | 35 |

In some aspects of the disclosure, the amino acid sequence of the peptide or peptidomimetic comprises VMAPRTLFL (SEQ ID NO:36) or VMAPRT(L/V)(V/L/I/F)L with one or more amino acid substitutions. In some embodiments, the amino acid sequence of the peptide or peptidomimetic comprises wherein one or more amino acids that are substituted with a Cys, Lys, Tyr, His, Ser, or Th.

In some embodiments, the amino acid sequence of the peptide or peptidomimetic comprises a substitution of the V residue at a position 1; a substitution of the M residue at a position 2; a substitution of the A residue at a position 3; a substitution of the P residue at a position 4; a substitution of the R residue at a position 5; a substitution of the T residue at a position 6; a substitution of the L residue at a position 7; a substitution of the F residue at a position 8; a substitution of the L residue at a position 9; or a combination of any of the foregoing substitutions. In some embodiments, the substitution is at position 1 and the amino acid is substituted for a 4Af, hAr, Ala, Dff, Ser, Msn, Gln, Cha, or Arg. In some embodiments, the substitution is at position 3 and the amino acid is substituted for a 4A, hAr, Ala, Dff, Ser, Msn, Gln, Cha, or Arg. In some embodiments, the substitution is at position 4 and the amino acid is substituted for a 4Af, hAr, Ala, Dff, Ser, Msn, Gln, Cha, or Arg. In some embodiments, the substitution is at position 5 and the amino acid is substituted for an Ala, Cha, Tha, or Mff. In some embodiments, the substitution is at position 7 and the amino acid is substituted for a 4Af, hAr, Ala, Dff, Ser, Msn, Gln, Cha, or Arg. In some embodiments, the substitution is at position 8 and the amino acid is substituted for a Arg, Msn, or hAR. In some embodiments, the substitution is at position 3 and the amino acid is substituted for a Cys. Lys, Tyr, His, Ser, or Thr. In some embodiments, the substitution is at position 8 and the amino acid is substituted for a Cys, Lys, Tyr, His, Ser, or Thr.

In another aspect of the disclosure, the synthetic peptides and peptidomimetics are designed to bind HLA-E. In some embodiments, the synthetic peptides and peptidomimetics bind HLA-E with low, medium, or high affinity. In some embodiments, the synthetic peptides and peptidomimetics bind HLA-E with higher affinity than VL9. In some embodiments, the synthetic peptides and peptidomimetics are covalently bound to HLA-E. In some embodiments, the covalent bond is between the synthetic peptides and peptidomimetics and HLA-E residues Tyr-7. His-9. Ser-24, Tyr-59, Arg-62, Glu-63, Ser-66, Thr-70, Gln-72, Asn-77, Thr-80, Tyr-84, Trp-97, His-99, Glu-114, Tyr-123, Trp-133, Ser-143, Lys-146, Ser-147, Glu-152, His-155, Gln-156, Tyr-159, Thr-163, Cys-164, Trp-167, or Tyr-171 of human HLA-E. In some embodiments, the covalent bond is between the synthetic peptides and peptidomimetics and HLA-E residues Tyr-7, Lys-146. Tyr-159, or Tyr-171 of human HLA-E.

In another aspect of the disclosure, the synthetic peptides and peptidomimetics are listed in TABLE 13, TABLE 14, TABLE 15, TABLE 16, TABLE 17, TABLE 18, or TABLE 19.

### Peptide Libraries

In some aspects, the disclosure is directed to libraries of synthetic peptides and peptidomimetics disclosed herein. In some embodiments, the peptide library has the design X-Met-X-X-Z-Ala-X-U-Leu (SEQ ID NO: 3) or X-Met-X-X-Z-Ala-X-U-Leu-Lys (SEQ ID NO: 4) is generated, wherein X=Gly, Ala, Met, Pro, Cpa, Cha, Ser, Asn, Gln, Msn, Phe, Tyr, His, Trp, 4Py, 4Af, Tha, Dff, Asp, Glu, Lys, Arg, hAr, Aad; Z=Gly, Ala, Val, Leu, Met, Pro, Cpa, Cba, Cha, Aoa, Phe, Trp, Mff, Dff, Tff, Tha, Nal, hPh, Dmf, Php, Amb and U=Ser, Thr, Asn, Gln, Msn, Hyp, Asp, Glu, Lys, Arg, Dab, Orn, Aad, hAr. In some embodiments, the half of the library has a Lys at the C-terminus. In some embodiments, anchor residues Met2 and Leu9 are set constant in the library design. In some embodiments, Arg5 is substituted with aliphatic and aromatic monomers, and Phe8 is replaced by polar and charged residues. In some embodiments, 21 non-canonical amino acids (shown in TABLE 2) are included in the library design.

In some embodiments the library is 1 million, 2 million, 3 million, 4 million, 5 million, 6 million, 7 million, 8 million, 9 million, or 10 million peptides in size. In some embodiments the library is 10 million, 20 million, 30 million, 40 million, 50 million, 60 million, 70 million, 80 million, 90 million, or 100 million, or 200 million peptides in size.

### III. Modifications

The central limitation in the development of peptide therapeutics is their short circulation time resulting from rapid enzymatic degradation and renal clearance. Methods to evade renal elimination by increasing the molecular weight have emerged, but extensive modifications can cause undesired steric hindrance during target binding. For small molecules, an alternative approach to modulate pharmacokinetic profiles and improve the potency and selectivity of a potential drug is the exploitation of covalent binding. Stability issues in peptides can be addressed via various strategies such as cyclization, incorporation of D- and non-canonical amino acids, and backbone modifications. A therapeutic small molecule ligand equipped with an electrophilic warhead binds covalently to nucleophilic groups of the target protein in a proximity-driven reaction. Irreversible covalent inhibition of an interaction can results in increased potency, selectivity, sustained pharmacodynamics, and could alleviate the effects of fast renal elimination. Therapeutic peptides may benefit from a covalent binding mode of action and alleviate pharmacokinetic limitations of this class of therapeutics.

In some aspects, the disclosure is directed to synthetic peptides and peptidomimetics that are chemically modified. In some embodiments, the peptide or peptidomimetic that is modified is selected from TABLE 3, TABLE 4, TABLE 5, or TABLE 6. In some embodiments, the peptide or peptidomimetic that is modified is selected from SEQ ID NO: 1-36. Modifications may comprise chemical modifications for example such as warheads, protective groups, and pegylation. In some embodiments, the modification is at the N- or C-terminus of the peptide or peptidomimetic. In some embodiments, the modification is on a side chain of an amino acid in the peptide or peptide. In some embodiments, the modification is on a Cys, Lys, Tyr, His, Ser, or Thr. In some embodiments, the modification is an arylation. In some embodiments, the arylation is on a Cys, Lys, Tyr, His, Ser, or Thr. In some embodiments, the modification is acetyladion, formylation, propanoylation, hexanoylation, or myristoylation. In some embodiments, the modification is an amidated C-terminus. In some embodiments, the modification is a substitution of one or more L-amino acid with a D-amino acid. In some embodiments, the modification is a substitution of one or more amino acid with a methyl-amino acid. In some embodiments, the modification is a substitution of an α-amino acid with a β-amino acids. In some embodiments, the arylation is on a Cys, and the Cys is a position 3 or 8 of the synthetic peptide or peptidomimetic.

### Warheads

In some aspects, the disclosure is directed to synthetic peptides and peptidomimetics comprising a warhead. In some embodiments, the peptide or peptidomimetic that is modified with a warhead is selected from TABLE 3, TABLE 4, TABLE 5, or TABLE 6. In some embodiments, the peptide or peptidomimetic that is modified is selected from SEQ ID NO: 1-36. In some embodiments, the warhead facilitates a covalent bond to a cognate protein after a chemical reaction. In some embodiments, the warhead is at the N- or C-terminus of the peptide or peptidomimetic. In some embodiments, the warhead is on a side chain of an amino acid in the peptide or peptide. In some embodiments, the warhead is on a Cys, Lys, Tyr, His, Ser, or Thr. In some embodiments, the warhead connected to the peptide by an arylation. In some embodiments, the arylation is on a Cys, Lys, Tyr, His, Ser, or Thr. In some embodiments, the warhead is on a Cys, and the Cys is a position 3 or 8 of the peptide or peptidomimetic. In some embodiments, the warhead is conjugated to the Cys via the Sulfur atom of the Cys.

In some embodiments, the peptide or peptidomimetic with a warhead has the following formula

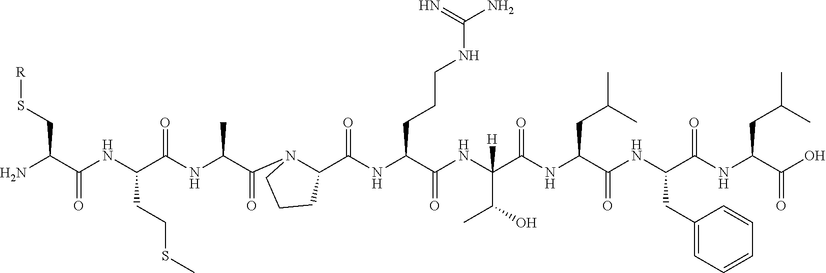

In some embodiments, the peptide or peptidomimetic with a warhead has the following formula

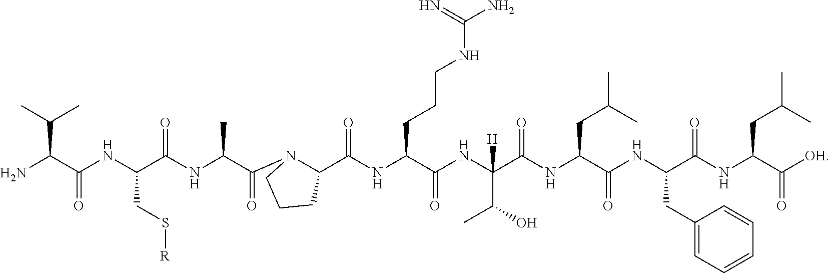

In some embodiments, the peptide or peptidomimetic with a warhead has the following formula

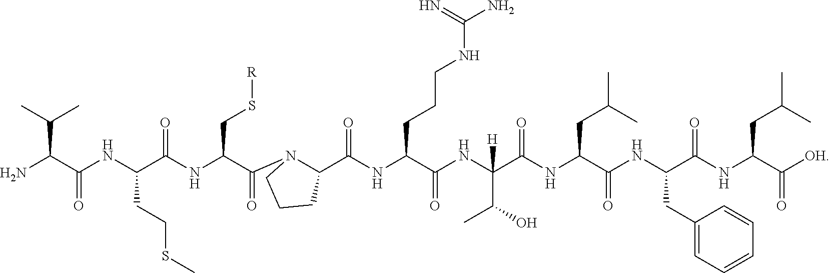

In some embodiments, the peptide or peptidomimetic with a warhead has the following formula

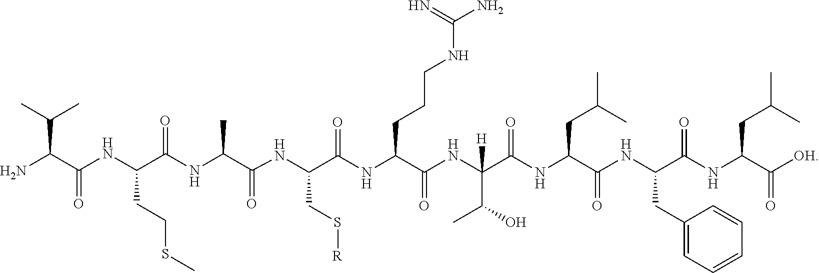

In some embodiments, the peptide or peptidomimetic with a warhead has the following formula

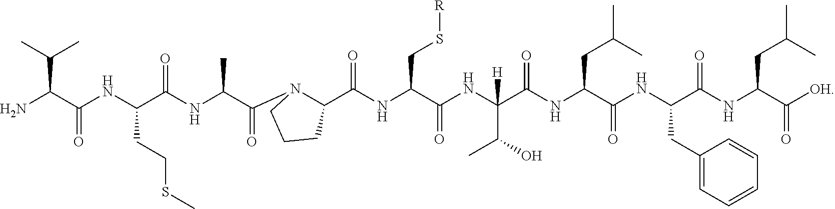

In some embodiments, the peptide or peptidomimetic with a warhead has the following formula

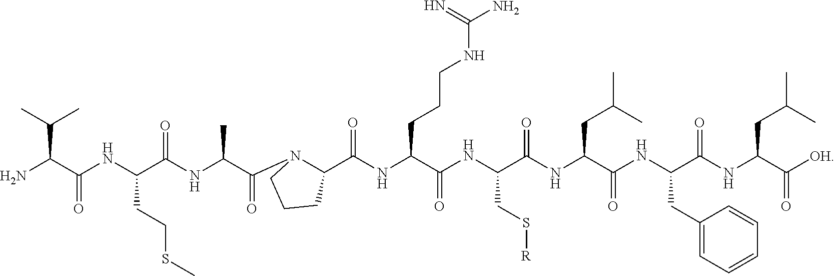

In some embodiments, the peptide or peptidomimetic with a warhead has the following formula

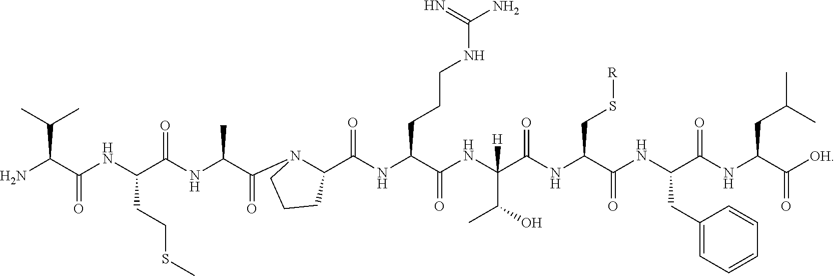

In some embodiments, the peptide or peptidomimetic with a warhead has the following formula

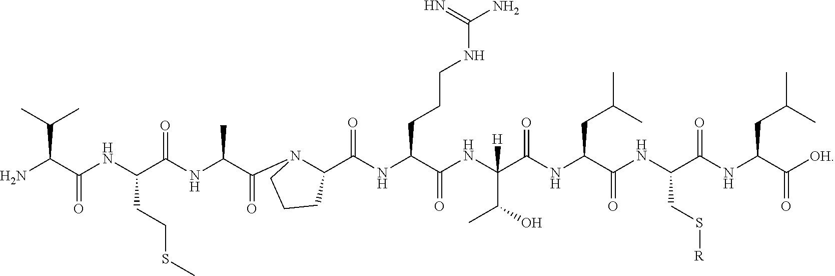

In some embodiments, the peptide or peptidomimetic with a warhead has the following formula

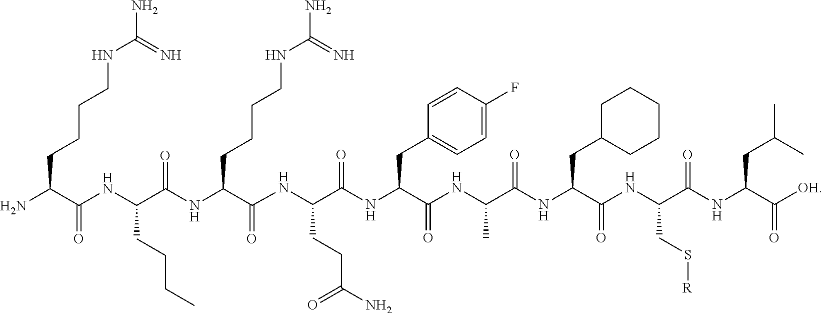

In some embodiments, the warhead is

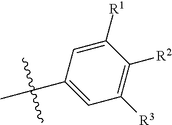

wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In some embodiments, the warhead is

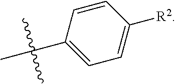

In some embodiments, the warhead is

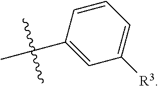

In some embodiments, the warhead is

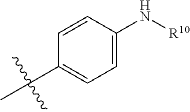

wherein R¹⁰ is hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂—N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

In some embodiments, the warhead is

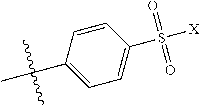

wherein X is a halogen.

In some embodiments, the warhead is

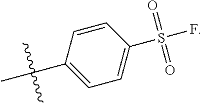 [SMILES: *c1ccc(S(=O)(=O)F)cc1]

In some embodiments, the warhead is selected from the group consisting of

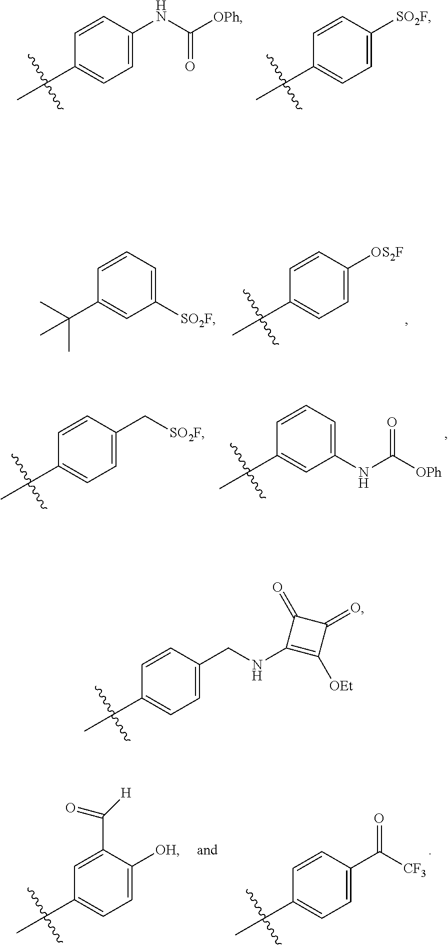

In some embodiments, the warhead is selected from the group of a sulfonyl fluoride, a phenyl carbamate, and a squareamate. In some embodiments, the warhead is conjugated to a Cys via the Sulfur atom of the Cys. Exemplary Cys substituted peptides and peptidomimetic sequences with a warhead are listed in TABLE 7. In some embodiments, the Cys substituted peptide with a warhead is selected from the group of SEQ ID NOs: 37-46.

TABLE 7 lists exemplary amino acid sequences of peptide and peptidomimetics with a warhead disclosed herein.

TABLE 7

| Amino Acid Sequence | SEQ ID NO: |
|---|---|
| NH₂-Cys(mSF)-Met-Ala-Pro-Arg-Thr-Leu-Phe-Leu-OH | 37 |
| NH₂-Val-Cys(mSF)-Ala-Pro-Arg-Thr-Leu-Phe-Leu-OH | 38 |
| NH₂-Val-Met-Cys(mSF)-Pro-Arg-Thr-Leu-Phe-Leu-OH | 39 |
| NH₂-Val-Met-Ala-Cys(mSF)-Arg-Thr-Leu-Phe-Leu-OH | 40 |
| NH₂-Val-Met-Ala-Pro-Cys(mSF)-Thr-Leu-Phe-Leu-OH | 41 |
| NH₂-Val-Met-Ala-Pro-Arg-Cys(mSF)-Leu-Phe-Leu-OH | 42 |
| NH₂-Val-Met-Ala-Pro-Arg-Thr-Cys(mSF)-Phe-Leu-OH | 43 |
| NH₂-Val-Met-Ala-Pro-Arg-Thr-Leu-Cys(mSF)-Leu-OH | 44 |
| NH₂-Val-Met-Ala-Pro-Arg-Thr-Leu-Phe-Cys(mSF)-OH | 45 |
| NH₂-hAr-Nle-hAr-Gln-Mff-Ala-Cha-Cys(mSF)-Leu-OH | 46 |

In some embodiments, the peptide or peptidomimetic with a warhead has the following formula

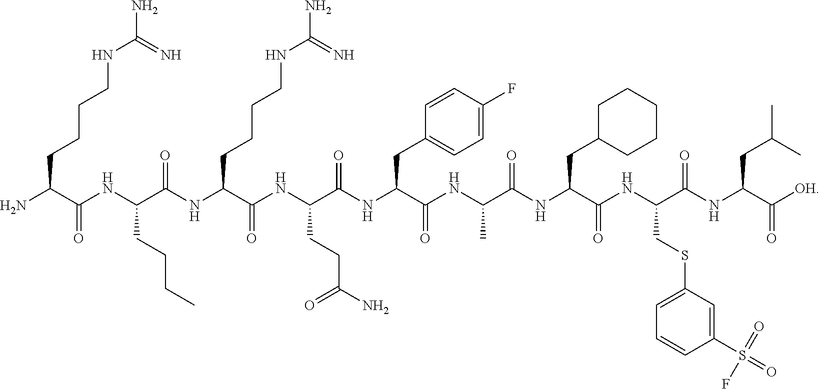 [SMILES: CCCC[C@H](NC(=O)[C@@H](N)CCCCNC(=N)N)C(=O)N[C@@H](CCCCNC(=N)N)C(=O)N[C@@H](CCC(N)=O)C(=O)N[C@@H](Cc1ccc(F)cc1)C(=O)N[C@@H](C)C(=O)N[C@@H](CC1CCCCC1)C(=O)N[C@@H](CSc1cccc(S(=O)(=O)F)c1)C(=O)N[C@@H](CC(C)C)C(=O)O]

In some embodiments, the peptide or peptidomimetic with a warhead has the following formula

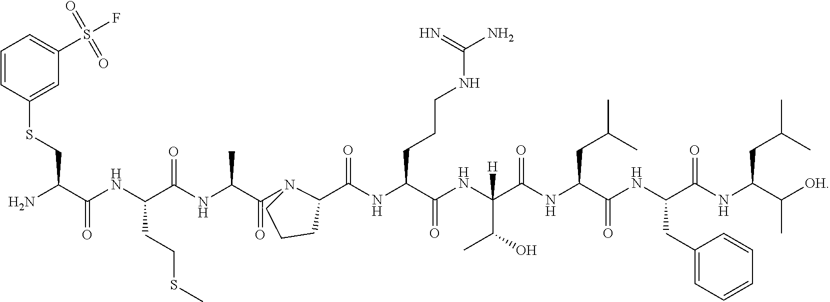 [SMILES: CSCC[C@H](NC(=O)[C@@H](N)CSc1cccc(S(=O)(=O)F)c1)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(C)O)[C@@H](C)O]

In some embodiments, the peptide or peptidomimetic with a warhead has the following formula

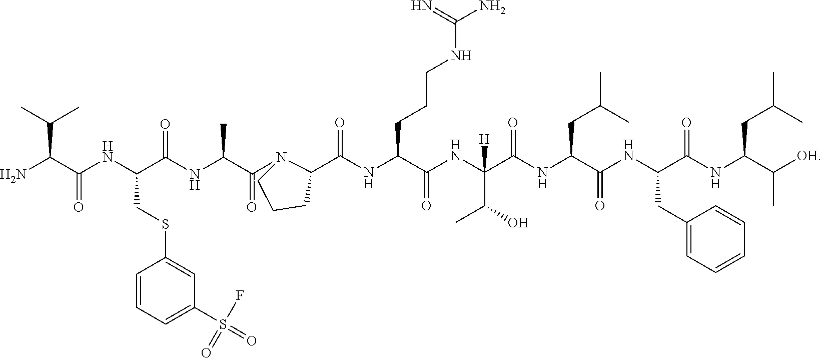 [SMILES: CC(C)C[C@H](NC(=O)[C@@H](NC(=O)[C@H](CCCNC(=N)N)NC(=O)[C@@H]1CCCN1C(=O)[C@H](C)NC(=O)[C@H](CSc1cccc(S(=O)(=O)F)c1)NC(=O)[C@@H](N)C(C)C)[C@@H](C)O)C(=O)N[C@@H](Cc1ccccc1)C(=O)N[C@@H](CC(C)C)C(C)O]

In some embodiments, the peptide or peptidomimetic with a warhead has the following formula

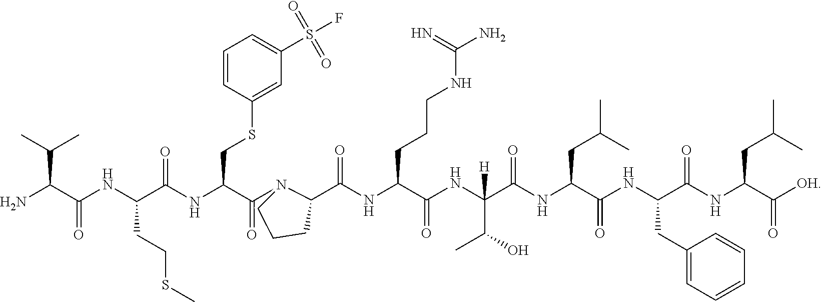 [SMILES: CSCC[C@H](NC(=O)[C@@H](N)C(C)C)C(=O)N[C@@H](CSc1cccc(S(=O)(=O)F)c1)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In some embodiments, the peptide or peptidomimetic with a warhead has the following formula

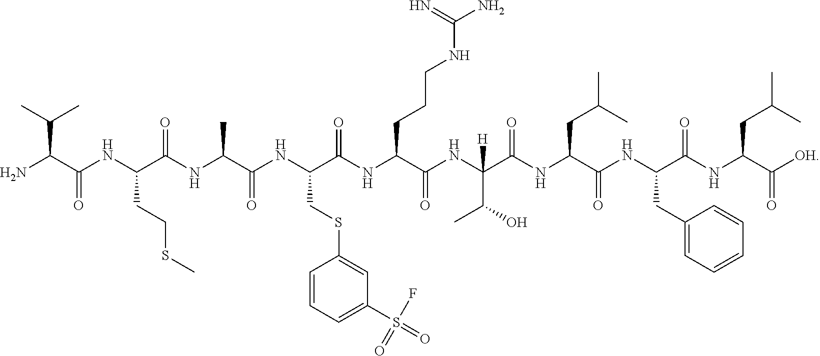 [SMILES: CSCC[C@H](NC(=O)[C@@H](N)C(C)C)C(=O)N[C@@H](C)C(=O)N[C@@H](CSc1cccc(S(=O)(=O)F)c1)C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In some embodiments, the peptide or peptidomimetic with a warhead has the following formula

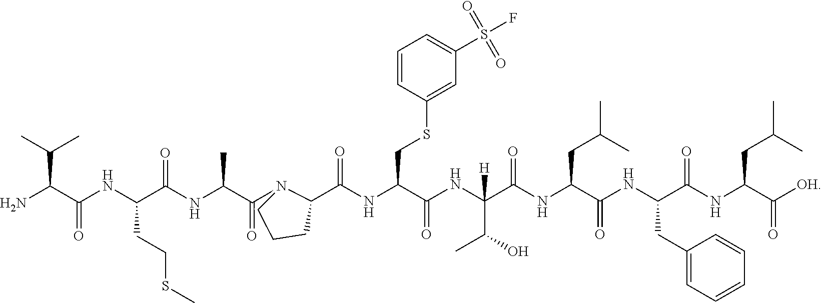 [SMILES: CSCC[C@H](NC(=O)[C@@H](N)C(C)C)C(=O)N[C@@H](C)C(=O)N1CCC[C@H]1C(=O)N[C@@H](CSc1cccc(S(=O)(=O)F)c1)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

In some embodiments, the peptide or peptidomimetic with a warhead has the following formula

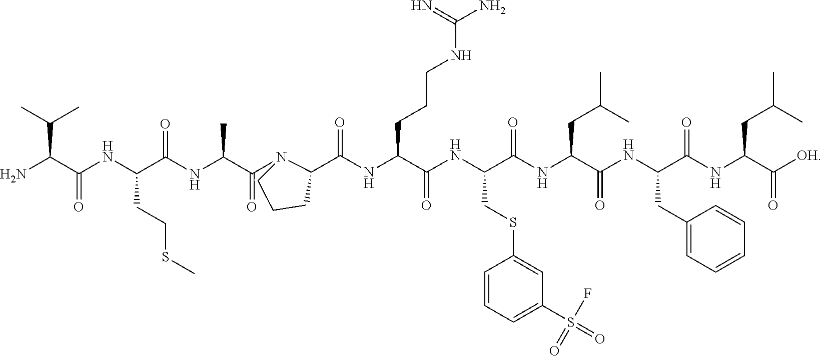 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@@H](CSc1cccc(S(=O)(=O)F)c1)C(=O)N[C@@H](CC(C)C)C(=O)N[C@@H](Cc1ccccc1)C(=O)N[C@@H](CC(C)C)C(=O)O]

In some embodiments, the peptide or peptidomimetic with a warhead has the following formula

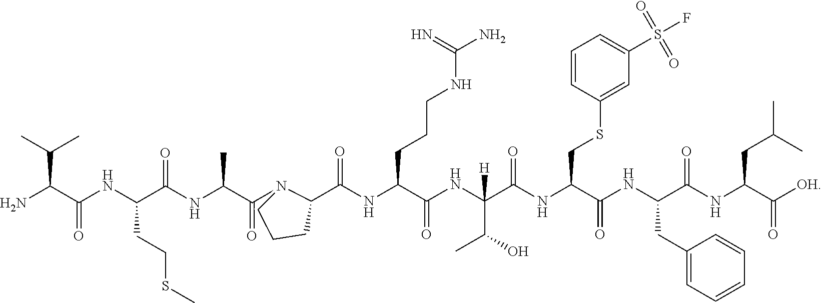 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CSc1cccc(S(=O)(=O)F)c1)C(=O)N[C@@H](Cc1ccccc1)C(=O)N[C@@H](CC(C)C)C(=O)O)[C@@H](C)O]

In some embodiments, the peptide or peptidomimetic with a warhead has the following formula

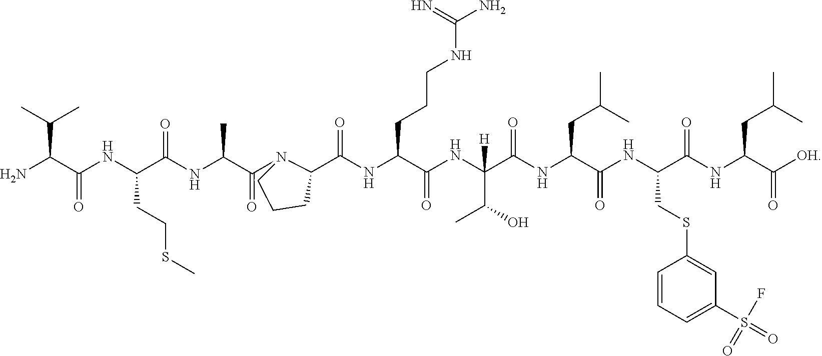 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](CSc1cccc(S(=O)(=O)F)c1)C(=O)N[C@@H](CC(C)C)C(=O)O)[C@@H](C)O]

### IV. Complexes

In some aspects, the disclosure is directed to synthetic peptides and peptidomimetics that are bound in a complex with HLA-E/β2m to form peptide/HLA-E/β2m. In some embodiments, the peptide or peptidomimetic that is complexed with HLA-E/β2m is selected from TABLE 3, TABLE 4, TABLE 5, TABLE 6, or TABLE 7. In some embodiments, the peptide or peptidomimetic that is complexed with HLA-E/β2m is selected from SEQ ID NOs: 1-47.

In some embodiments, the synthetic peptides and peptidomimetics are designed to bind HLA-E/β2m. In some embodiments, the synthetic peptides and peptidomimetics bind HLA-E/β2m with low, medium, or high affinity. In some embodiments, the synthetic peptides and peptidomimetics bind HLA-E with higher affinity than VL9. In some embodiments, the peptides and peptidomimetics are covalently bound to HLA-E/β2m. In some embodiments, the covalent bond is between the synthetic peptides and peptidomimetics and HLA-E residues Tyr-7, His-9, Ser-24, Tyr-59, Arg-62, Glu-63, Ser-66, Thr-70, Gln-72, Asn-77, Thr-80, Tyr-84, Trp-97, His-99, Glu-114, Tyr-123, Trp-133, Ser-143, Lys-146, Ser-147, Glu-152, His-155, Gln-156, Tyr-159, Thr-163, Cys-164, Trp-167, or Tyr-171 of human HLA-E. In some embodiments, the covalent bond is between the synthetic peptides and peptidomimetics and HLA-E residues Tyr-7, Lys-146, Tyr-159, or Tyr-171 of human HLA-E.

In some embodiments, the peptide/HLA-E/β2m is modulated in binding of CD94/NKG2A. In some embodiments, the peptide/HLA-E/β2m is inhibited in binding or engaging of CD94/NKG2A or prevents activation of CD94/NKG2A. In some embodiments, the peptide/HLA-E/β2m is located on a cell. In some embodiments, the peptide/HILA-E/β2m is soluble. In some embodiments, the cell is a cancer cell. In some embodiments, the CD94/NKG2A is located on a NK cell or a T cell. In some embodiments, the inhibition of binding or engaging of peptide/HLA-E/β2m complex to CD94/NKG2A on a NK cell or a T cell modulates activity of the NK cell or the T cell.

### V. Preparation of Peptides

Methods for producing synthetic peptide or peptidomimetic of the disclosure are known in the art such as solid phase peptide synthesis (SPPS), Fmoc-based synthesis, and Boc-based synthesis by an automatic peptide synthesizer. For example, peptides can be chemically synthesized using the sequence information provided herein and using peptide synthesis methods known in the art. The produced synthetic peptide or peptidomimetic can be modified during or after peptide synthesis with several modifications, for example with a warhead, a protective group, or pegylation. Alternatively or additionally, the peptide or peptidomimetic may be modified at its amino terminus or carboxy terminus or protected by various organic groups for protecting the peptide from protein-cleaving enzymes in vivo while increasing its stability. The produced synthetic peptide or peptidomimetic can then be purified further. Purification strategies for peptides or peptidomimetics are known in the art, and include FPLC and HPLC based methods.

### VI. Pharmaceutical Compositions

For therapeutic use, a synthetic peptide or peptidomimetic disclosed herein preferably is combined with a pharmaceutically acceptable carrier and/or an excipient. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing a synthetic peptide or peptidomimetic disclosed herein can be presented in a dosage unit form and can be prepared by any suitable method. A pharmaceutical composition should be formulated to be compatible with its intended route of administration, e.g., oral administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

The composition can be formulated as a solution, microemulsion, dispersion, liposome, or other ordered structure suitable for stable storage at high concentration. Sterile solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

### VII. Methods of Use

The synthetic peptides and peptidomimetics of the disclosure can be used in a variety of in vitro and in vivo methods, as research reagents, for diagnostic purposes, and for therapeutic uses, based on the binding specificity of the synthetic peptides and peptidomimetics to HLA-E and on the effect on HLA-E functions of the peptides and peptidomimetics.

Approximately half of peripheral NK cells display the CD94/NKG2A receptor and these cells are mostly present in the CD56high fraction, which contains the more immature cells. Intratumoral NK cells have somewhat higher frequencies of CD94/NKG2A. CD94/NKG2A is also expressed on intratumoral CD8+ T cells that often display a late effector memory phenotype. The inhibitory signals induced by NKG2A receptor engagement with peptide/HLA-E results in decreased capacity of NK cells and CD8+ T cells to lyse target cells. It is contemplated, that disrupting the HLA-E-CD94/NKG2A axis may lead to reversal of the inhibitory effect that leads to immune tolerance in these cells.

The synthetic peptides and peptidomimetics disclosed herein are designed to be recognized and bound covalently or non-covalently by HLA-E/β2m complexes. In some embodiments, the synthetic peptides and peptidomimetics can be used to modulate HLA-E/β2m function. In some embodiments, the synthetic peptides and peptidomimetics in complex with HLA-E modulate HLA-E engagement with the CD94/NKG2A receptor heterodimer on NK or T cells. In some embodiments, the synthetic peptides and peptidomimetics in complex with HLA-E block or inhibit HLA-E engagement with the CD94/NKG2A receptor heterodimer on NK or T cells. In some embodiments, the HLA-E/β2m complex is presented on the surface of a cancer cell and the peptide or peptidomimetic binds to the HLA-E/β2m complex in a manner that blocks the HLA-E/β2m complex from engaging with the CD94/NKG2A receptor on NK or T cells.

Methods for testing for peptide/HLA-E/β2m-CD94/NKG2A engagement and subsequent cell signaling are known in the art, for example by FACS, cytotoxicity assays, and cytokine release assays.

### VIII. Kits

In some embodiments, any of the synthetic peptides or peptidomimetics disclosed herein disclosed herein is assembled into a pharmaceutical or diagnostic or research kit to facilitate their use in therapeutic, diagnostic or research applications. A kit may include one or more containers housing any of the systems or vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

### EXAMPLES

Below are examples of specific embodiments for carrying out what is disclosed herein. The examples are offered for illustrative purposes only and are not intended to limit scope.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, PROTEINS: STRUCTURES AND MOLECULAR PROPERTIES (W.H. Freeman and Company, 1993); A. L. Lehninger, BIOCHEMISTRY (Worth Publishers, Inc., current addition); Sambrook, et al. MOLECULAR CLONING: A LABORATORY MANUAL (2nd Edition, 1989); METHODS IN ENZYMOLOGY (S. Colowick and N. Kaplan eds., Academic Press, Inc.); REMINGTON'S PHARMACEUTICAL SCIENCES, 18th Edition (Easton, Pennsylvania: Mack Publishing Company, 1990); Carey and Sundberg ADVANCED ORGANIC CHEMISTRY 3^{rd} Ed. (Plenum Press) Vols A and B (1992).

Unless otherwise stated, all reagents and chemicals were obtained from commercial sources and used without further purification.

### Example 1—Peptide Library Design and Synthesis

This example describes the design of a peptide library based on the HLA-E signal peptide VL9 VMAPRT(L/V)(V/L/I/F)L (SEQ ID NO:1) (VMAPRTLVL, SEQ ID NO:2) and subsequent library synthesis of a library of 200 million peptides synthesized by split-and-pool synthesis.

Briefly, the interaction between HLA-E and VL9 with CD94/NKG2A was analyzed in an HLA-E/VL9/CD94/NKG2A complex crystal structure (the crystal structure is represented in PDB ID 3cii). In the crystal structure, VL9 binds the groove formed by two α-helices of HLA-E and several amino acids within VL9 were found crucial for anchoring to HLA-E (Met2, Leu9) and binding to the receptor dimer (Arg5, Phe8). To discover peptides that occupy the binding groove of VL9 in HLA-E but do not enable binding of the CD94-NKG2A receptor dimer, a focused library of 9-mer peptides with the formula X1-Met2-X3-X4-Z5-Ala6-X7-U8-Leu9 (SEQ ID NO:3) and 10-mer peptides X1-Met2-X3-X4-Z5-Ala6-X7-U8-Leu9-Lys10 (SEQ ID NO:4) (where X=Gly, Ala, Met, Pro, Cpa, Cha, Ser, Asn, Gln, Msn, Phe, Tyr, His, Trp, 4Py, 4Af, Tha, Dff, Asp, Glu, Lys, Arg, hAr, Aad; Z=Gly, Ala, Val, Leu, Met, Pro, Cpa, Cba, Cha, Aoa, Phe, Trp, Mff, Dff, Tff, Tha, Nal, hPh, Dmf, Php, Amb (21 aliphatic or aromatic amino acids); and U=Ser, Thr, Asn, Gln, Msn, Hyp, Asp, Glu, Lys, Arg, Dab, Orn, Aad, hAr (charged or polar amino acids)). Half of the library had an extra amino acid (a Lys, Lys10) at the C-terminus at position 10. Canonical and non-canonical amino acids used in the peptide library are listed in TABLE 1, TABLE 2, and shown in FIG. 1. The anchor residues Met2 and Leu9 were set constant in the library design, to enable binding to HLA-E. Substitution of Thr6 with Ala was previously shown to reduce binding to CD94/NKG2A. According to the library design, Arg5 was substituted with aliphatic and aromatic amino acids, and Phe8 was replaced by polar and charged amino acids to antagonize binding to CD94/NKG2A. C-terminal Lys was installed on half of the library to increase sequencing confidence by augmented signal intensity of fragments in secondary mass spectra. To increase the chemical diversity of the peptide collection, the 21 non-canonical amino acids were included in the library design.

A focused library of ˜2×10⁸ 9- and 10-mer peptides was synthesized by split-and-pool synthesis (SPPS) on monosized resin.

Briefly, a peptide library with 200 million members was synthesized on Tentagel® M NH₂ resin (30 μm bead size, 0.72 mmol, 2.79 g, 70 million beads/g). The resin was placed in a fritted syringe and swollen in N,N-dimethylformamide (DMF) for 30 min. To the resin was added 4-(4-Hydroxymethyl-3-methoxyphenoxy)butyric acid (HMPB, 7 equiv.) with 1-[Bis(dimethylamino)methylene]-1H-1,2,3-triazolo[4,5-b]pyridinium 3-oxide PF₆ (HATU, 6.3 equiv.) and N,N-diisopropylethylamine (DIEA, 21 equiv.) in DMF for 30 min. The reactants were removed by washing with DMF (3×), and the resin was split into two parts to couple the C-terminal amino acids Fmoc-Leu-OH, and Fmoc-Lys(Boc)-OH for the 9-mer and 10-mer library members, respectively. The amino acids (7 equiv.) were dissolved in DMF (0.5 M), N,N′-diisopropylcarbodiimide (DIC, 5 equiv.) was added, and the solution was added to the resin after 2 min of activation. 4-Dimethylaminopyridine (DMAP, 0.1 equiv.) was added, and the reaction was left for 16 h. The Fmoc protecting group was removed with 20% piperidine in DMF (2×5 min). Fmoc-Leu-OH (5 equiv.) was coupled to the resin functionalized with Lys with HATU (4.5 equiv) and Dipea (15 equiv) in DMF for 15 min, and, after washing and Fmoc deprotection, the resins were combined for split and pool synthesis was continued by coupling amino acids (7 equiv.) by activation with HATU (6.3 equiv.) and DIEA (21 equiv.) for 15 min at RT followed by Fmoc deprotection according to the library design. After final deprotection and extensive washing by DMF and CH₂Cl₂, the resin was dried in a vacuum chamber for 16 h. Peptides were cleaved from solid support using 60 mL trifluoroacetic acid/H₂O/1,2-ethanedi-thiol/triisopropylsilane (TFA/H₂O/EDT/TIPS; 94:2.5:2.5:1) for 2 h at RT. TFA was evaporated to 20% of the initial volume by applying a stream of nitrogen, and the library was precipitated by addition of ice-cold Et₂O. The suspension was centrifuged (4000 rpm, 5 min, 5° C.), and the residue was subjected to two more rounds of precipitation and centrifugation. After evaporation of residual Et₂O, the precipitate was dissolved in 30% MeCN in H₂O (+0.1% TFA) and lyophilized. The crude library lyophilizate was dissolved in 5% MeCN in H₂O (+0.1% TFA) for solid phase extraction using Supelclean™ LC-18 SPE Tubes (100 mg crude library per gram of resin bed). The purified library was lyophilized and dissolved with phosphate-buffered saline (PBS)+10% DMF to a concentration of 4 mM (20 pM per library member) for storage as single-use aliquots of 1 mL at −80° C.

### Example 2—Protein Expression and Purification

This example describes the expression, purification, and refolding of HLA-E and 02m protein from inclusion bodies in E. coli and the expression and purification of CD94/NKG2A single-chain dimer from mammalian cells.

Preparation of HLA-E and B2M Proteins from Inclusion Bodies

The coding sequences for HLA-E*0103 (human, residues 22-305)(SEQ ID NO:48) with a C-terminal Avitag and β2m (human, residues 21-119) (SEQ ID NO:49) were synthesized and cloned into pET29b(+) (pET29b(+)-HLAE*(hu)(22-305)-Avitag and pET29b(+)-(β2m (h)(21-119)).

The proteins were expressed in E. coli BL21 (DE3) at 37° C. until an OD₆₀₀ of 0.7 and then induced with 1.0 mM IPTG for 3 hours at 37° C. For purification of proteins from inclusion bodies, pellets from 10 L cultures were resuspended in 200 mL of sucrose buffer (50 mM Tris pH 8.0, 1 mM EDTA and 25% sucrose), lysed with the addition of 0.2 g of lysozyme. After 10 minutes of lysis the solution was diluted with 500 mL of deoxycholate solution (20 mM Tris pH 7.5, 100 mM NaCl, 1% deoxycholic acid, and 1% Triton). The mixture was then adjusted to 5 mM MgCl₂ and treated with 4 mg of DNAse (Sigma D-5025) until viscosity was reduced to that of water. Inclusion bodies were pelleted at 8K×g for 20 minutes after DNAse treatment and in between subsequent washes. Pellets were washed 3 times with Triton solution (50 mM Tris pH 8.0, 100 mM NaCl, 1 mM EDTA, and 0.5% Triton X-100) and further 3 times with Tris solution (50 mM Tris pH 8.0, 100 mM NaCl, and 1 mM EDTA). Finally, pellets were resuspended in urea solution (25 mM MES pH 6.0, 8 M urea, 10 mM EDTA, and 0.1 mM DTT). Protein concentrations were determined by A₂₈₀ using extinction coefficients for individual proteins.

### Refolding of Biotinylated HLA-E Complexes

MHC complexes were refolded as previously described in Braud et al., Nature 1998, 391 (6669), 795-799 and Altman et al., Science 1996, 274 (5284), 94-96. Briefly, 34.7 mg of [HLAE(hu)(22-305)]-Avitag and 23.7 mg [1B2M(h)(21-119)] were refolded with 30 mg of either VMAPRTLFL (VL9) (SEQ ID NO: 36) to form HLA-E+VL9 or VMAP(Anpp)TLFL (SEQ ID NO: 47) (UV-labile VL9, VL9^{UV}) to form HLA-E+VL9^{UV} by dilution into 1 L of refolding buffer (400 mM L-arginine, 100 mM Tris, pH 8.0, 2 mM EDTA, 5 mM reduced glutathione, and 0.5 mM oxidized glutathione). Prior to addition, each protein was diluted with 4 mL of injection buffer (3 M guanidine HCl, 10 mM sodium acetate, and 10 mM EDTA). An additional 34.7 mg [HLAE(hu)(22-305)]-Avitag, diluted in injection buffer was added twice more at 12-hour intervals. Refolding solution was then subjected to buffer exchange by tangential flow filtration over a 30K MWCO PALL Omega TFF Cassette into 20 mM Tris pH 8.0. MHC complexes were then purified on an AKTA PURE using a 5 mL HiTrap Q FF column with a 25 CV gradient from 0 to 500 mM NaCl. Fractions were pooled corresponding to the refolded complex and dialyzed into 20 mM Tris pH 8.0. Complexes were then biotinylated with BirA Ligase (Avidity) and subjected to buffer exchange into TBS (20 mM Tris pH 7.5, 150 mM NaCl). Protein concentrations were determined using the extinction coefficient of the MHC complexes.

### CD94-NKG2A Single-Chain Dimer Expression and Purification

A single-chain dimer of CD94/NKG2A (hSCD) was generated by fusing human CD94 (K32-I179, Uniprot Q13241) via a GS(G4S)₇GG linker to human NKG2A (P94-L333, Uniprot P26715) (SEQ ID NO:50). An N-terminal 8×His tag was added for IMAC purification, and the whole construct was subcloned into a mammalian expression vector with a puromycin selection cassette. In a second construct (oaFc-hSCD)(SEQ ID NO:51), the N-terminal 8×His tag was replaced by an AviTag™ followed by a one-armed human IgG1 Fc (L234A, L235A, L351K, T366S, P395V, F405R, Y407A, K409Y), linked to the hSCD insert with a GGG linker. Stable cell lines for both constructs were generated in HEK293F cells using puromycin selection. Cells were grown in Expi293 Expression Media (Thermo A1435101), and the protein was purified from 1-2 L of conditioned media at a cell density of ˜3×10⁶ cells/mL. Conditioned media was collected by centrifugation at 3000 rpm for 20 min at 4° C. and filtered through a 0.2 uM filter unit (Corning 430515). For the his-tagged hSCD, the filtered conditioned media was loaded onto a 5 mL HisTrap™ FF column (Cytiva, 17-5255-01) on an Akta FLPC at 1 mL/min flow rate. The column was then washed with 10 CV of HBS (50 mM HEPES, 300 mM NaCl, pH 7.5) with 20 mM imidazole. The protein was eluted using a gradient elution of 20 mM-1 M imidazole in HBS over 6 CV. Fractions were pooled, concentrated, and passed through a Superdex® 200 Increase 10/300 GL column (Sigma GE28-9909-44) in HBS. For oaFC-hSCD purification, the filtered conditioned media was loaded on a 5 mL HiTrap™ Protein G HP (Cytiva, 17-0404-01) on an Akta FPLC at 1 mL/min flow rate. The column was then washed with 10 CV of PBS (Corning 21-040-CV). The protein was eluted using 0.1 M acetic acid (pH 2.7) and immediately neutralized with 1:10 the volume of 1 M Tris pH 8 and 1 M NaCl. Peak fractions were pooled, concentrated, and injected onto a Superdex® 200 Increase 10/300 GL column (Sigma GE28-9909-44) equilibrated in PBS running at 0.5 mL/min. The main peak was collected and further concentrated for downstream applications. The final protein concentration was quantified using Pierce™ 660 nm Protein Assay Reagent (Thermo 22660) before the protein was aliquoted, flash frozen in liquid nitrogen, and stored at −80° C. until use.

### Example 3—Peptide Selection by Binding to HLA-E

This example describes the selection of HLA-E peptide binders from the focused library by nano liquid chromatography-tandem mass spectrometry (nLC-MS/MS).

Affinity selections with HLA-E bound to UV-labile VL9 ([HLA-E+VL9^{UV}]; [HLAE(hu)(22-305)]-Avitag+BM(h)(21-119)]+VMAP(Anpp)TLFL]*Biotin) were performed following adapted procedures for discovery of peptides from ultra-large peptide libraries described by Quartararo et al. Nature Communications 2020, 11 (1), 3183.

Briefly, purified biotinylated HLA-E-B2M complex as described in EXAMPLE 2 was pre-charged with a UV-cleavable peptide resulting in HLA-E+VL9^{UV}. Then affinity selections against [HLA-E+VL9^{UV}] immobilized on magnetic beads were performed with the focused library from EXAMPLE 1 at a concentration of 10 pM per member on a 1 mL scale (10 fmol/peptide). MyOne Streptavidin T1 DynaBeads (10 mg/mL; 1 mg; 0.13 nmol protein binding capacity, 1 equiv.) were functionalized with biotinylated [HLA-E+VL9^{UV}] or off-target control protein (0.156 nmol, 1.2 equiv.) in wash buffer (PBS (+10% FCS, +0.02% Tween 20)) in a 1.7 mL microcentrifuge tube on a nutating mixer for 30 min at 4° C. For washing, the beads were subjected to three cycles of suspending in 1 mL wash buffer followed by separation enabled by a magnetic rack. The washed beads were suspended in PBS (+10% FCS), and the library was added a concentration of 10 pM/member in 1.7 mL centrifuge tubes. Selections were performed under UV irradiation to cleave UV-labile VL9 and liberate the binding groove of HLA-E in presence of the peptide library. The tubes were placed on a nutating mixer and irradiated by a handheld UV lamp in 3 cm distance (λ=366 nm) for 1 h at 4° C. After incubation, the solution was removed on the separating rack, and the beads were subjected to three cycles of wash with PBS and separation enabled by the magnetic rack. Finally, the beads were treated with 6 M guanidine in 0.2 M phosphate buffer (pH 6.8) to denaturate the proteins and elute bound peptides. The samples were desalted by a C18 ZipTip prior to lyophilization, and dissolved in 100 mM guanidine in H₂O (+0.1% formic acid) for analysis by nano liquid chromatography-tandem mass spectrometry (nLC-MS/MS) on an Orbitrap Fusion Lumos Tribrid Mass Spectrometer.

nLC-MS/MS

Briefly, samples from affinity selections were analyzed on a Thermo Fisher Orbitrap Fusion Lumos Tribrid Mass Spectrometer with an EASY-Spray source using a Thermo Fisher EASY-nLC 1200 System and Acclaim™ PepMap™ 100 C18 trap columns (20 mm×75 μm, 3 μm particle size, 100 Å pore size, PN164946) and Acclaim™ PepMap™ RSLC C18 HPLC columns (150 mm×50 μm, 2 μm particle size, 100 Å pore size, PN ES901). LC was performed with 0.1% formic acid (FA) in water (solvent A) and 80% MeCN with 0.1% formic acid in water (solvent B) prepared with LiChrosolv® water and MeCN suitable for MS from Millipore Sigma and Optima™ LC/MS grade formic acid from Thermo Fisher Scientific. Chromatography was performed at 40° C., with a flow rate of 300 nL/min using either of the following gradient: 1% B to 45% B (0-100 min), 45% B to 90% B (100-102 min), 90% B (102-100 min) or 1% B to 51% B (0-120 min), 51% B to 90% B (120-130 min), 90% B (130-140 min). 5 minutes after start of the gradient, MS/MS were recorded in a data-dependent method. Full MS cycle time=3 s. Detector Type=Orbitrap. Resolution=120000. Mass Range=Normal. Quadrupole Isolation=True. Scan Range (m/z)=200-1400. RF Lens (%)=30. AGC Target=250%. Maximum Injection Time=Auto. The following filters were applied for precursor selection: Monoisotopic Precursor Selection=Peptides. Precursor Selection Range (m/z)=200-1400. Intensity Threshold=4.0e4. Charge States=2-10. Dynamic Exclusion (exclusion after 1n for 30 s, mass tolerance=10 ppm). Fragmentation was induced by collision-induced dissociation (CID), higher-energy collisional dissociation (HCD), and electron-transfer dissociation with higher-energy collision (EThcD). Specifications CID: Isolation Mode=Quadrupole. Isolation Window (m/z)=1.3. Isolation Offset=off. CID Collision Energy (%)=30, 10 ms Activation Time. Activation Q=0.25. Detection=Orbitrap. Orbitrap Resolution=30000. Mass Range=Normal. Scan Range Mode=Auto. AGC Target=Standard. Maximum Injection Time=Auto. 1 Microscan, Centroid Data, no Internal Calibration. Specifications HCD: Isolation Mode=Quadrupole. Isolation Window (m/z)=1.3. Isolation Offset=Off. Collision energy (%)=25. Detection=Orbitrap. Resolution=30000. Mass Range=Normal. 1 Microscan, Centroid Data, no Internal Calibration. Specifications EThcD: Isolation Mode=Quadrupole. Isolation Window (m/z)=1.3. Isolation Offset=Off. Use Calibrated Charge-Dependent ETD Parameters=True. ETD Supplemental Activation=EThcD. SA Collision Energy=25%. Detection=Orbitrap. Orbitrap Resolution=30000. Mass Range=Normal. Scan Range Mode=Auto. AGC Target=Standard. Maximum Injection Time=Auto. 1 Microscan, Centroid Data, no Internal Calibration. ThermoFisher Xcalibur software package and PEAKS Studio 8.5 were used for data analysis.

Selections with [HLA-E+VL9^{UV}] and the control protein were performed in triplicates.

### De Novo Sequencing and Hit Identification

PEAKS Studio (V8.5, Bioinformatics Solutions) was used to process raw nLC-MS/MS data and perform de novo peptide sequencing. Automated de novo sequencing was performed with a 15 ppm parent mass error tolerance, and a 0.02 Da fragment mass error tolerance. The following variable post-translation modifications were defined to resolve peptides with non-canonical amino acids: Cpa=Val+12.00; Cba=Val+26.02; Cha=Phe=6.05; 4Py=Phe +1.00; 4Af=Phe+15.01; Tha=His+16.96; Mff=Phe+17.99; Dff=Phe+35.98; Tff=Phe +53.97; Msn=Met+31.99; hAr=Arg+14.02; Aad=Glu+14.02; Aoa=Leu+28.03; Nal=Phe+50.02; hPh=Phe+14.02; Amb=Gly+76.03; Dmf=Phe+60.02; Php=Pro+90.05; Orn=Val+11.07; Hyp=Pro+15.99; Dab=Gly+43.04; Met(oxide)=Met+15.99. Up to 20 candidates were reported per scan. Data cleaning and hit identification was performed as previously described in Vinogradov et al., ACS Combinatorial Science 2017, 19 (11), 694-701.

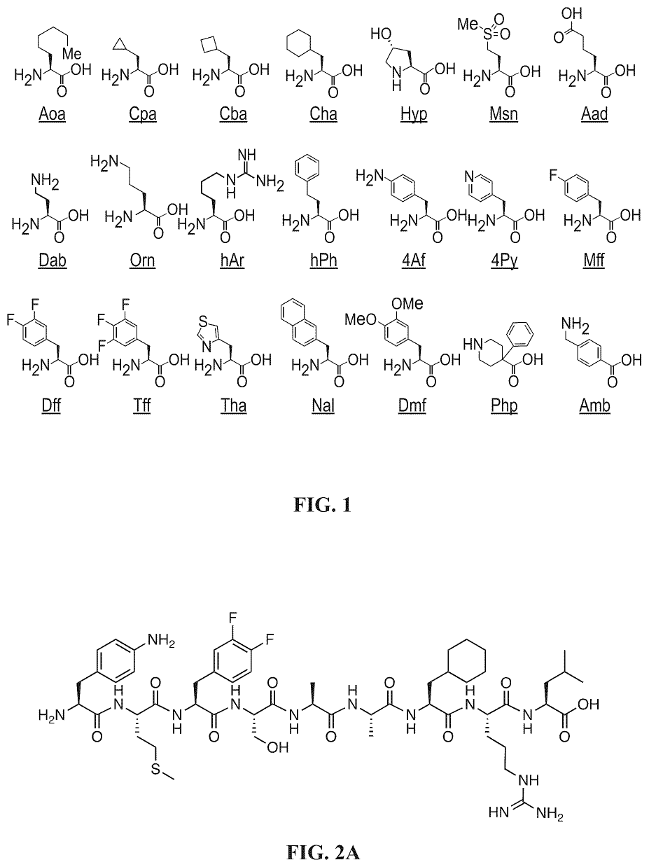

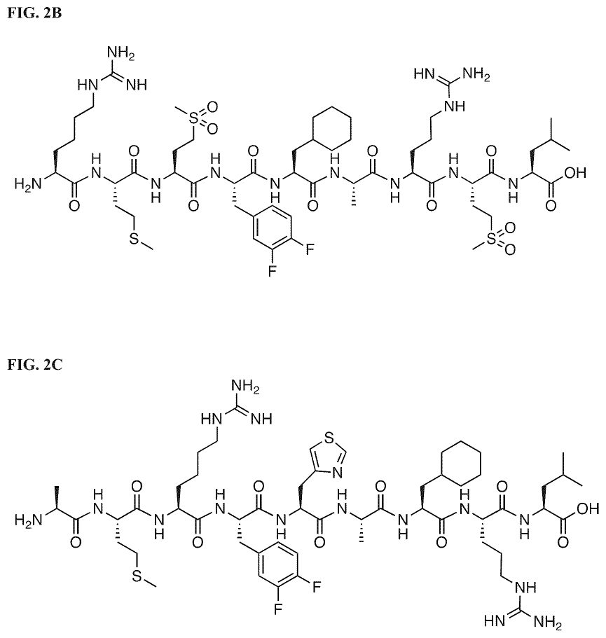

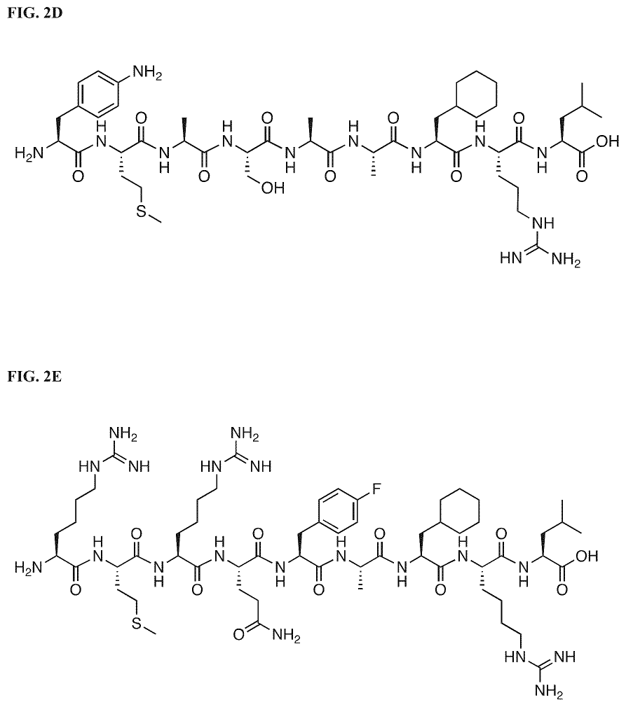

After de novo sequencing of the fragmented ions and data filtering, five nonameric peptides matching the library design were identified with high average local confidence (ALC) scores and displayed selective binding to HLA-E. The sequences of the five 9-mer peptides, B1, B2, B3, B4, and B5 are listed in TABLE 8. Exemplary formulas for the peptides B1-B5 are shown in FIGS. 2A-2F.

TABLE 8

| | | SEQ |
|---|---|---|
| Peptide | Amino Acid Sequence | ID NO: |
| B1 | NH2-4Af-Met-Dff-Ser-Ala-Ala-Cha-Arg-Leu-OH | 5 |
| B2 | NH2-hAr-Met-Msn-Dff-Cha-Ala-Arg-Msn-Leu-OH | 6 |
| B3 | NH2-Ala-Met-hAr-Dff-Tha-Ala-Cha-Arg-Leu-OH | 7 |
| B4 | NH2-4Af-Met-Ala-Ser-Ala-Ala-Cha-Arg-Leu-OH | 8 |
| B5 | NH2-hAR-Met-hAR-Gln-Mff-Ala-Cha-hAR-Leu-OH | 9 |

No 10-mer peptide was discovered with selective binding, indicating that the C-terminal Lys extension might be unfavorable for binding HLA-E.

Additional peptides identified in the selective binding assay are shown in TABLE B.

TABLE B

| SEQ ID NO: | Peptide |
|---|---|
| 52 | AMkhfAcRL |
| 53 | aMShWAekL |
| 54 | AvDaMAcRL |
| 55 | AvlSmAvSL |
| 56 | avShWAekL |
| 57 | cMDcLAfDL |
| 58 | cMjkLAhlL |
| 59 | cvDcLAfDL |
| 60 | DMAciAlQL |
| 61 | DMWfVAljL |
| 62 | DvAciAlQL |
| 63 | eMAaoAdEL |
| 64 | eMASAAcRL |
| 65 | eMFSAAcRL |
| 66 | eMhSAAcRL |
| 67 | evASAAcRL |
| 68 | evFSAAcRL |
| 69 | evhDAAcRL |
| 70 | evhSAAcRL |
| 71 | FMAYAAlRL |
| 72 | fMYdnASRL |
| 73 | fvkfcAaRL |
| 74 | hMAYFAdEL |
| 75 | hMAYFAeDL |
| 76 | hMEcmAlKL |
| 77 | hMYSiAlRL |
| 78 | hvaSiAHjL |
| 79 | hvAYFAdEL |
| 80 | hvAYFAeDL |
| 81 | hvEcmAlKL |
| 82 | hvlaFAdEL |
| 83 | jvAYFAdEL |
| 84 | kMAYFAdEL |
| 85 | kMjhcARjL |
| 86 | kMkQgAckL |
| 87 | kvAYFAdEL |
| 88 | kvAYFAjsL |
| 89 | kvjhcARjL |
| 90 | lMfaoAdEL |
| 91 | lMfaoAjsL |
| 92 | lvfaoAdEL |
| 93 | lvfaoAeDL |
| 94 | MMWcMAhkL |
| 95 | PMYdnARSL |
| 96 | PMYdnASRL |
| 97 | PvFamASKL |
| 98 | QMWjaAMkL |
| 99 | QvWjaAvkL |
| 100 | RMkfFAPkL |
| 101 | SMDaMAcRL |
| 102 | SMDavAcRL |
| 103 | SvDaMAcRL |
| 104 | SvDavAcRL |
| 105 | SvRcVAhkL |
| 106 | SvYhVAfsL |
| 107 | YMAYFAeDL |
| 108 | YvAYFAdEL |
| 109 | YvhSAAcRL |

a = Cpa, b = Cba, c = Cha, d = 4Py, e = 4Af, f = Tha, g = Mff, h = Dff, i = Tff, j = Msn, k = hAr, l = Aad, m = Aoa, Nal, o = hPh, p = Amb, q = Dmf, r = Php, s = Orn, t = Hyp, u = Dab, v = Met(O), canonical amino acids are represented by their one-letter code.

### Example 4—Inhibition of CD94-NKG2A Binding to HLA-E by Peptides

This example describes the analysis of inhibition of CD94-NKG2A binding to HLA-E by the peptides B1-B5 by bio-layer interferometry (BLI).

### Synthesis of Peptides

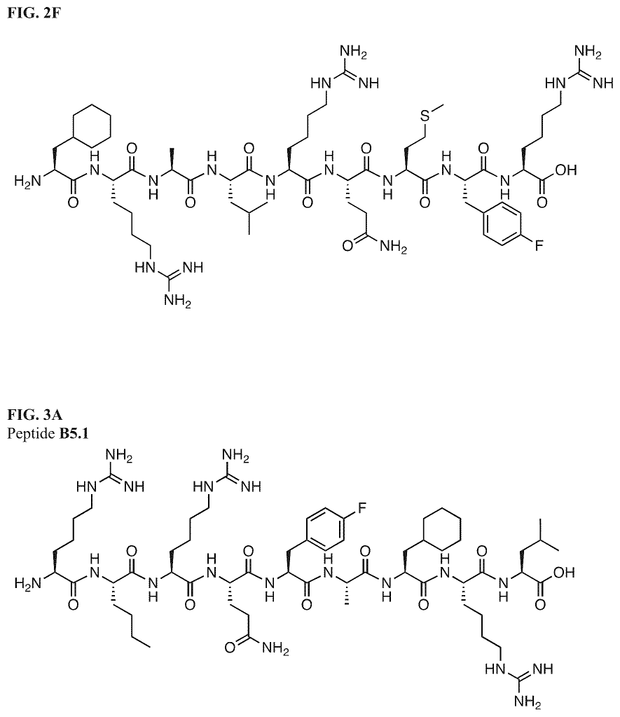

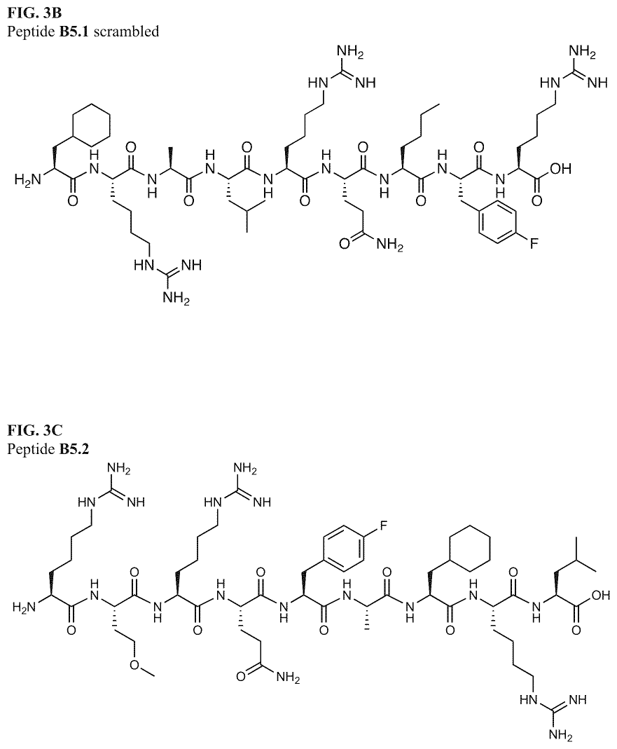

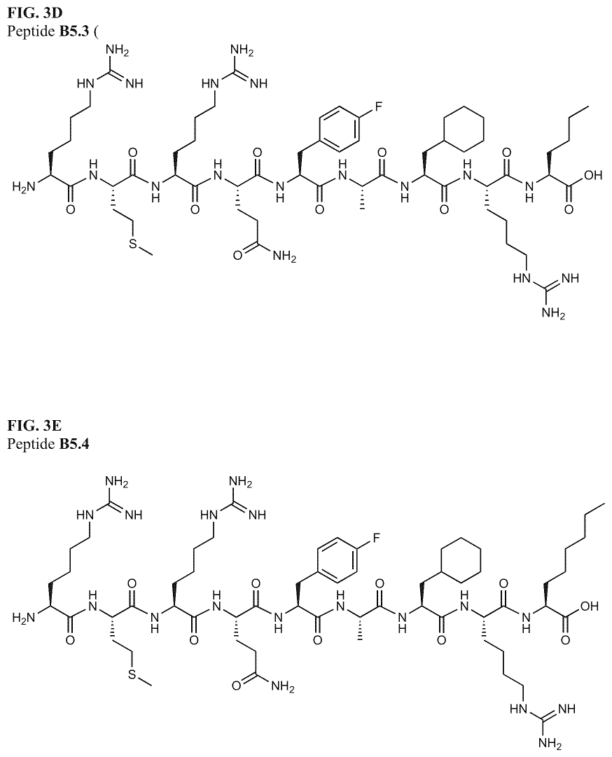

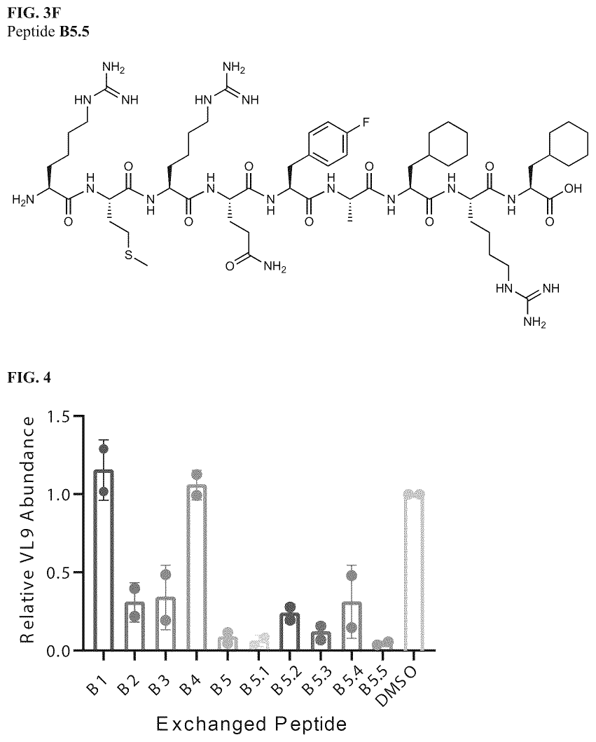

Peptides B1-B5 identified in EXAMPLE 3 and a series of peptides with single substitution of anchor residues Met2 or Leu9 of B5 (B5-1, B5-2, B5-3, B5-4, and B5-5, shown in TABLE 9 and FIGS. 3A-3F) and 2 scrambled control peptides (B5. Scrambled and B5.1 scrambled) were synthesized.

TABLE 9

| | | SEQ |
|---|---|---|
| Peptide | Sequence | ID NO: |
| B5.1 | NH₂-hAr-Nle-hAr-Gln-Mff-Ala-Cha-hAr- | 21 |
| | Leu-OH | |
| B5.2 | NH₂-hAr-Mox-hAr-Gln-Mff-Ala-Cha-hAr- | 22 |
| | Leu-OH | |
| B5.3 | NH₂-hAr-Met-hAr-Gln-Mff-Ala-Cha-hAr- | 23 |
| | Nle-OH | |
| B5.4 | NH₂-hAr-Nle-hAr-Gln-Mff-Ala-Cha-hAr- | 24 |
| | Aoa-OH | |
| B5.5 | NH₂-hAr-Nle-hAr-Gln-Mff-Ala-Cha-hAr- | 25 |
| | Cha-OH | |
| B5.scrambled | NH₂-Cha-hAr-Ala-Leu-hAr-Gln-Met-Mff- | 110 |
| | hAr-OH | |
| B5.1.scrambled | NH₂-Cha-hAr-Ala-Leu-hAr-Gln-Nle-Mff- | 111 |
| | hAr-OH | |

Briefly, peptides were synthesized by SPPS on a 0.05 mmol scale with a HMPB ChemMatrix resin in fritted syringes. The C-terminal amino acid (7 equiv.) was coupled to the resin by DIC (5 equiv.) and DMAP (0.1 equiv.) for 16 h at rt. The Fmoc protecting group was removed using 20% piperidine in DMF for 2×5 min, and the subsequent amino acids (5 equiv.) were coupled using HATU (4.5 equiv.) and DIEA (15 equiv.) in DMF for 15 min at room temperature (rt). Upon completion of the sequence and deprotection of the N-terminal amine, the resin was washed with DMF and CH₂Cl₂, and dried in a vacuum chamber for 16 h. Cleavage from solid support and global deprotection was achieved with TFA/H₂O/EDT/TIPS (94:2.5:2.5:1) for 2 h at rt. The solution was concentrated to 10% of its initial volume by a stream of nitrogen, and the peptides were isolated by three cycles of precipitation by ice-cold Et₂O and centrifugation. The dried, crude peptides were dissolved in 30% MeCN (+0.1% TFA) and lyophilized.

Crude peptides were dissolved in 10% MeCN in water (+0.1% TFA) and purified by reverse-phase HPLC or reverse-phase flash chromatography. The purity of fractions was determined by LC-MS, and pure fractions were pooled and lyophilized. The synthesis yield (%) was calculated as pure isolated material divided by theoretical amount (based on synthesis scale) adjusted for the fraction of crude material used for purification. Exemplary yields are listed in TABLE 10.

TABLE 10

| Peptide | Total Yield | Calculated and LC MS Mass |
|---|---|---|
| B1 | 4.8 mg, 3.5 μmol, | Exact mass: calculated for |
| | 39% yield | C₅₃H₈₁F₂N₁₃O₁₁S: 1145.59 |
| | | Da, found: 1145.60 Da |
| B2 | 8.0 mg, 5.2 μmol, | Exact mass: calculated for |
| | 20% yield | C₅₅H₉₃F₂N₁₅O₁₄S₃: 1321.62 |
| | | Da, found: 1321.61 Da |
| B3 | 8.2 mg, 5.7 μmol, | Exact mass: calculated for |
| | 38% yield | C₅₄H₈₆F₂N₁₆O₁₀S₂: 1220.61 |
| | | Da, found: 1220.61 Da |
| B4 | 7.3 mg, 5.8 μmol, | Exact mass: calculated for |
| | 39% yield | C₄₇H₇₉N₁₃O₁₁S: 1033.57 |
| | | Da, found: 1033.58 Da |
| B5 | 7.5 mg, 4.6 μmol, | Exact mass: calculated for |
| | 31% yield | C₅₈H₁₀₀FN₁₉O₁₁S: 1289.76 |
| | | Da, found: 1289.76 Da |
| B5 | 2.0 mg, 1.2 μmol, | Exact mass: calculated for |
| scrambled | 9% yield | C₅₈H₁₀₀FN₁₉O₁₁S: 1289.76 |
| | | Da, found: 1289.90 Da |
| B5.1 | 8.8 mg, 5.5 μmol, | Exact mass: calculated for |
| | 27% yield | C₅₉H₁₀₂FN₁₉O₁₁: 1271.80 |
| | | Da, found: 1271.95 Da |
| B5.1 | 15.7 mg, 9.7 μmol, | Exact mass: calculated for |
| scrambled | 18% yield | C₅₉H₁₀₂FN₁₉O₁₁: 1271.80 |
| | | Da, found: 1271.80 Da |
| B5.2 | 6.1 mg, 3.8 μmol, | Exact mass: calculated for |
| | 18% yield | C₅₈H₁₀₀FN₁₉O₁₂: 1273.78 |
| | | Da, found: 1273.93 Da |
| B5.3 | 1.9 mg, 1.2 μmol, | Exact mass: calculated for |
| | 6% yield | C₅₈H₁₀₀FN₁₉O₁₁S: 1289.76 |
| | | Da, found: 1289.90 Da |
| B5.4 | 9.7 mg, 5.8 μmol, | Exact mass: calculated for |
| | 29% yield | C₆₀H₁₀₄FN₁₉O₁₁S: 1317.79 |
| | | Da, found: 1317.94 Da |
| B5.5 | 5.9 mg, 3.5 μmol, | Exact mass: calculated for |
| | 18% yield | C₆₁H₁₀₄FN₁₉O₁₁S: 1329.79 |
| | | Da, found: 1329.94 Da |

### Peptide Exchange on Refolded HLA-E Complexes

The biotinylated complex of HLA-E and B2M with VL9 (abbreviated as [HLA-E+VL9]) was used to evaluate the potential of de novo discovered peptides B1-B5. Ligand exchange of the peptides B1-B5 with VL9 on HLA-E was studied by incubating [HLA-E+VL9] with the competitor peptides.

Briefly, 10 μM competitive peptides, VL9 peptide, or DMSO were added to 1 μM of refolded HLA-E/B2M complexes loaded with VL9 in 1×PBS (Corning 21-040-CV) and allowed to exchange for 4 h at 25° C.

### HLA-E Peptide Exchange Analysis by SEC and LC-MS

To determine the exchanged peptides in the HLA-E/B2M complexes, the exchanged complexes were analyzed by SEC and LC-MS. Briefly, an Agilent 1200 Series Infinity II HPLC coupled to an analytical fraction collector (Agilent, G1364F) was used to separate peptide-loaded HLA-E/B2M protein from free peptide after peptide exchange as described above. Briefly, 50 μL of peptide-exchange solution was loaded onto a Superdex 200 Increase size exclusion column (Cytiva, 28990945) at a flow rate of 0.45 mL/min in PBS at room temperature. The main UV absorbance peak at 280 nm was collected over a 200 μL volume fraction for downstream analysis. 20 μL of the collected fraction was injected into an Agilent 1200 Series Infinity II HPLC with a 2.1×50 mm ZORBAX 80 Å Extend-C18 reverse phase column (Agilent, Part Number: 727700-902) equilibrated in water with 0.1% (v/v) formic acid and 10% (v/v) acetonitrile flowing at 0.2 mL/min with a column temperature of 40° C. The peptide peaks were resolved using a 10-60% gradient of acetonitrile in 0.1% (v/v) formic acid and eluted into a Dual Agilent Jet Steam electrospray ionization source operating at a Gas Temp of 350° C., Drying Gas at 10 L/min, Nebulizer at 30 psig, Sheath Gas Temp at 350° C., Sheath Gas Flow at 11 L/min, VCap voltage at 3500 V, and Nozzle Voltage at 1000 V. Peptide ions were detected with an Agilent 6230 Time-of-Flight mass spectrometer operating in positive ion mode with a Fragmentor Voltage of 150 V and Skimmer Voltage of 65 V. Spectra were analyzed using MassHunter software version B.07. Extracted ion chromatograms of base peaks associated with VL9 (VMAPRTLFL) or competitive peptides B1-B5 were integrated, and the area under the curve was used for relative quantification of peptide exchange. Exemplary results are shown in FIG. 4. Peptides B2, B3, and B5 showed over 60% ligand exchange after 4 h of incubation. B5-1, B5-2, B5-3, B5-4, and B5-5) showed comparable exchange with VL9.

### Biolayer Interferometry (BLI)

The inhibitory potential of peptides B1-B5, and B5.1 was evaluated by measuring binding of CD94/NKG2A to HLA-E after incubation with the peptides. Proteins were produced as described in EXAMPLE 2 and peptides were synthesized as described in EXAMPLE 3. Biotinylated [HLA-E+VL9](1 μM) was co-incubated with individual peptides B1-B5 (10 PM) overnight at room temperature, and the biotinylated complex was subsequently loaded on streptavidin-coated probes for analysis by BLI. Binding inhibition of CD94-NKG2A (200 nM) to the HLA-E-loaded probes was determined relative to the parent [HLA-E+VL9] complex.

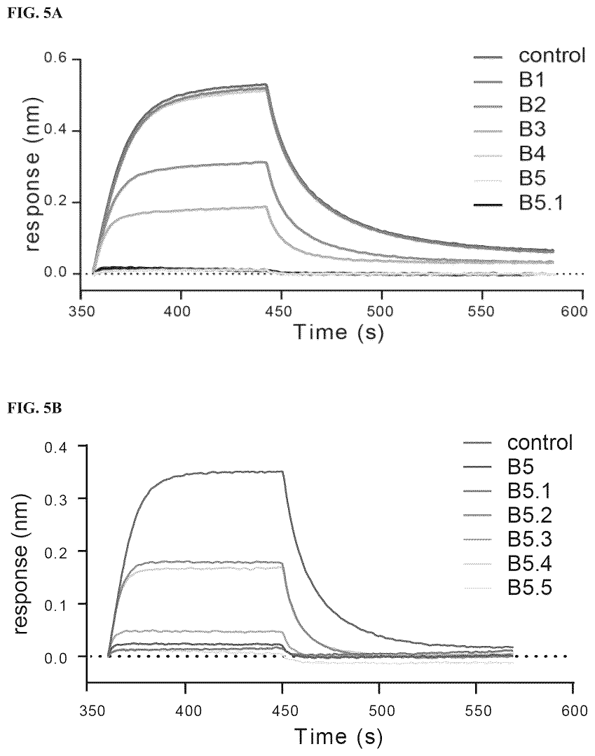

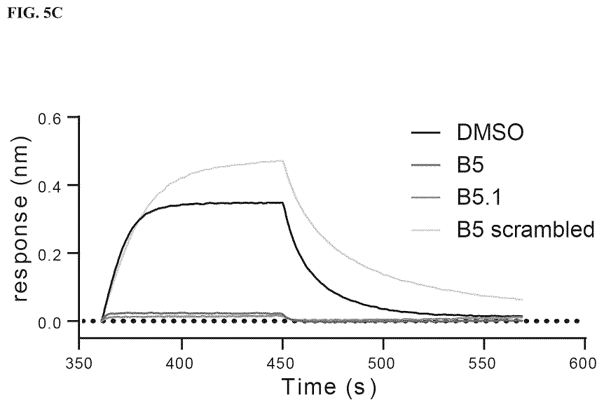

BLI was performed on a ForteBio Octet Red96e instrument. All proteins were diluted in 1× Kinetic Buffer (10× Kinetic buffer, Sartorius 181105) diluted in 1×PBS. A Blocking Buffer step was introduced to lower non-specific binding to the streptavidin (SA) tips by diluting 5% bovine serum albumin (BSA) and 20 μg/mL Biocytin (Sigma B4261) in 1× Kinetic Buffer. Refolded and biotinylated [HLA-E+VL9] complexes were loaded onto SA biosensors (Forte 18-5019) at ˜5 μg/ml (100 nM) with the following steps: 60 sec baseline in 1× Kinetic Buffer, 180 sec loading, 60 sec blocking in Blocking Buffer and 60 sec baseline in 1× Kinetic Buffer, all at 25° C. and 1000 rpm. hSCD and oaFC-hSCD binding to immobilized HLA-E/B2M complexes was monitored at 200 nM by a 90 sec association step, followed by a 120 sec dissociation step. Data correction was performed as follows: first, aligning the data to the average of the last baseline step on the y-axis; second, by aligning the data to the dissociation step for inter-step correction; and third by filtering the data using Savitzky-Golay Filtering. B2, B3, and B5 showed 41-98%. inhibition of CD94/NKG2A binding in the biophysical assay. In particular, B5 showed only 2% of residual binding of CD94/NKG2A compared to untreated control (FIG. 5A-5C).

A series of peptides with single substitution of anchor residues Met2 or Leu9 of B5 (B5-1, B5-2, B5-3, B5-4, and B5-5) showed comparable inhibition of CD94/NKG2A binding (FIG. FIG. 5A-5C). A Met/Nle substitution (peptide B5.1) prepared to avoid the formation of oxidative side-products during synthesis, purification, and handling demonstrated comparable activity in this assay. A scrambled analog of B5 (control) showed no inhibition of binding with CD94/NKG2A

### Example 5—Covalent Binding of VL9 Analogs to HLA-E

This example describes the introduction of electrophilic warheads into VL9 derived peptides and the covalent binding of the armed peptides to HLA-E.

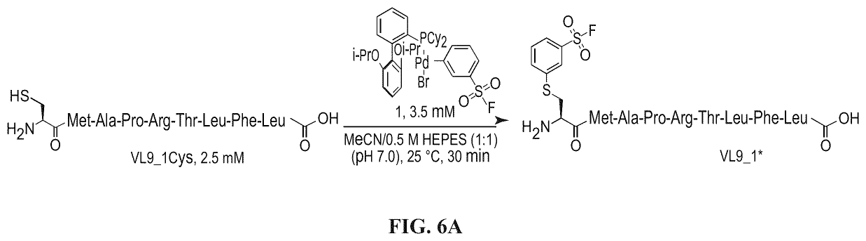

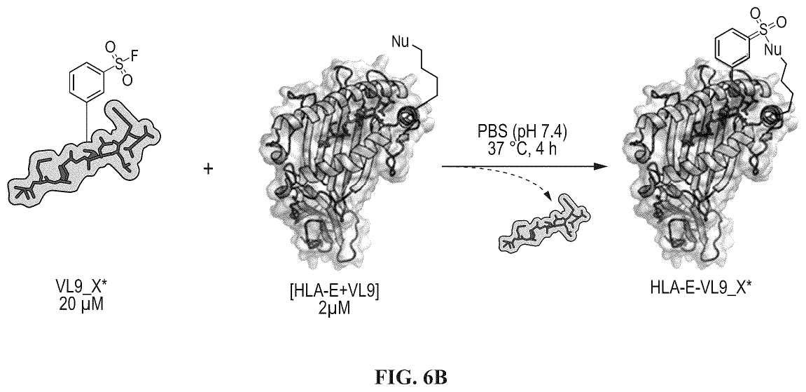

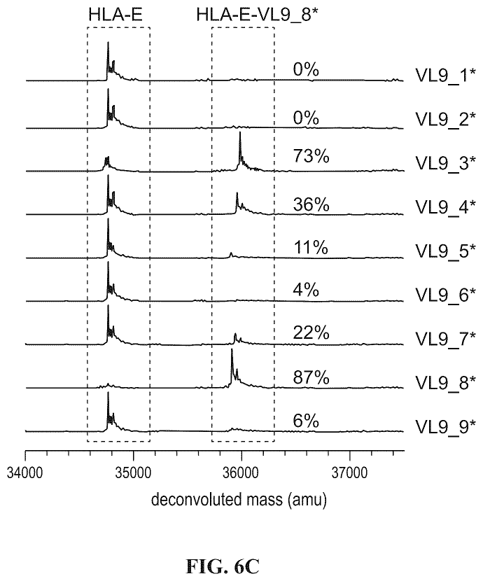

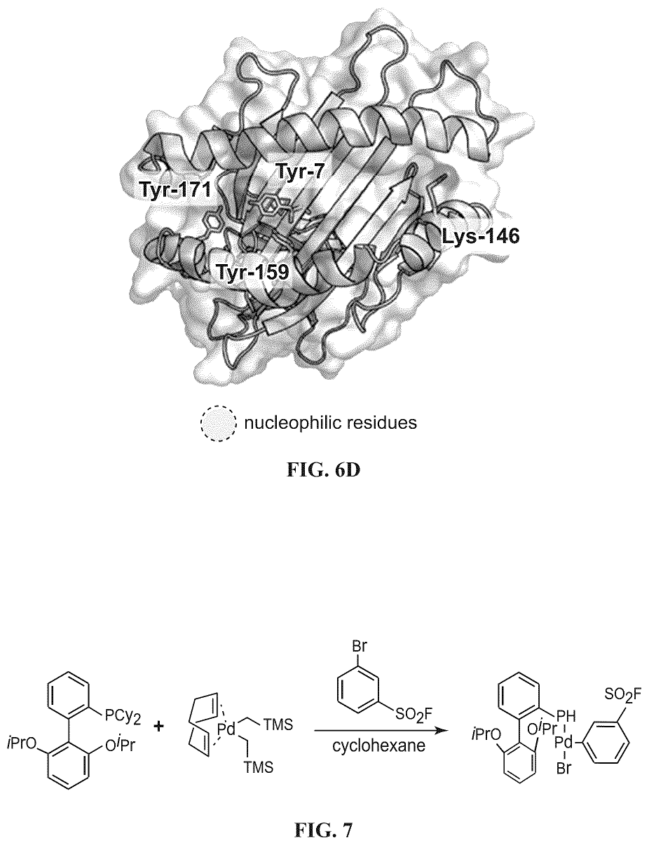

Covalent inhibition is a useful strategy to increase potency, selectivity, and pharmacodynamics of drugs, and alleviates the effects of fast renal elimination of peptides. Several residues in the proximity of VL9 in the binding groove of HLA-E, e.g., Tyr-7, Lys-146, Tyr-159, Tyr-171, bear nucleophilic groups potentially amenable for covalent binding through a Sulfur(VI) Fluoride Exchange (SuFEx) electrophile contained by the meta-substituted aryl sulfonyl fluoride (mSF) warhead (FIG. 6A).

To determine the optimal site on an HLA-E-binding peptide for efficient cross-linking with the target protein, an electrophile scan with VL9 the endogenous ligand of HLA-E was performed. A library of peptide variants of VL9 with single Cys mutations were synthesized for every position in the sequence. Additionally, peptide B5.1 (described in EXAMPLE 4) was equipped with an electrophilic warhead to further increase its potential as inhibitor of the HLA-E/CD94-NKG2A interaction. Position 8 of the B5.1 peptide (B5.1_8*) was selected for the installation of the aryl sulfonyl fluoride warhead, as this position led to the highest conversion in the electrophile scan with VL9. The sequences of the Cys substituted peptides VL9-1 Cys(mSF) to VL9-9 Cys(mSF) (also denoted as VL9-1* to VL9-9*) and B5.1_8 Cys(mSF) (also denoted as B5.1_8*) with the warhead position marked as mSF are shown in TABLE 11.

TABLE 11

| | | SEQ |
|---|---|---|
| Peptide | Amino Acid Sequence | ID NO: |
| VL9-1 | NH₂-Cys(mSF)-Met-Ala-Pro-Arg-Thr-Leu-Phe- | 37 |
| Cys(mSF) | Leu-OH | |
| VL9-2 | NH₂-Val-Cys(mSF)-Ala-Pro-Arg-Thr-Leu-Phe- | 38 |
| Cys(mSF) | Leu-OH | |
| VL9-3 | NH₂-Val-Met-Cys(mSF)-Pro-Arg-Thr-Leu-Phe- | 39 |
| Cys(mSF) | Leu-OH | |
| VL9-4 | NH₂-Val-Met-Ala-Cys(mSF)-Arg-Thr-Leu-Phe- | 40 |
| Cys(mSF) | Leu-OH | |
| VL9-5 | NH₂-Val-Met-Ala-Pro-Cys(mSF)-Thr-Leu-Phe- | 41 |
| Cys(mSF) | Leu-OH | |
| VL9-6 | NH₂-Val-Met-Ala-Pro-Arg-Cys(mSF)-Leu-Phe- | 42 |
| Cys(mSF) | Leu-OH | |
| VL9-7 | NH₂-Val-Met-Ala-Pro-Arg-Thr-Cys(mSF)-Phe- | 43 |
| Cys(mSF) | Leu-OH | |
| VL9-8 | NH₂-Val-Met-Ala-Pro-Arg-Thr-Leu-Cys(mSF)- | 44 |
| Cys(mSF) | Leu-OH | |
| VL9-9 | NH₂-Val-Met-Ala-Pro-Arg-Thr-Leu-Phe- | 45 |
| Cys(mSF) | Cys(mSF)-OH | |
| B5.1_8 | NH₂-hAr-Nle-hAr-Gln-Mff-Ala-Cha-Cys(mSF)- | 46 |
| Cys(mSF) | Leu-OH | |

Synthesis of Palladium Oxidative Addition Complex, (RuPhos)Pd(m-benzenefluorosulfonyl)Br, 1

To a 1 dram vial equipped with a magnetic stirbar was added 3-bromobenzenesulfonyl fluoride (33 mg, 0.14 mmol, 1.1 equiv) and RuPhos (65 mg, 0.14 mmol, 1.1 equiv). The vial was loosely sealed with a screw cap and brought into a nitrogen-filled glovebox. Cyclohexane (1.5 mL) and (cod)Pd(CH₂TMS)₂ (50 mg, 0.13 mmol, 1.0 equiv) were added in that order, resulting in a clear solution. The reaction vessel was sealed tightly, removed from the glovebox and allowed to stir at room temperature overnight. The reaction mixture was opened to atmosphere, pentane (1.5 mL) was added, and the mixture allowed to stand in at −20° C. for 2 h. The resulting precipitate was collected by vacuum filtration and washed twice with a minimal amount ice-cooled pentane. Drying under high vacuum afforded the desired product as a grey solid (38 mg, 33% yield), which was used without further purification. An exemplary synthesis diagram is shown in FIG. 7. The identity and integrity of the Palladium Oxidative Addition Complex was analyzed with ¹H NMR, ¹³C NMR, ³¹P NMR, and ¹⁹F NMR.

(¹H NMR (400 MHz, CD₂Cl₂) δ 7.71-7.57 (m, 4H), 7.55-7.44 (m, 2H), 7.44-7.33 (m, 1H), 7.17 (t, J=7.8 Hz, 1H), 6.89 (ddd, J=7.7, 3.1, 1.3 Hz, 1H), 6.68 (d, J=8.5 Hz, 2H), 4.64 (hept, J=6.0 Hz, 2H), 2.15-2.02 (m, 2H), 1.89-1.48 (m, 13H), 1.38 (d, J=5.8 Hz, 6H), 1.27-1.06 (m, 5H), 1.02 (s, 6H), 0.84 (s, 1H), 0.58 (s, 1H) ppm. 13C NMR (101 MHz, CD₂Cl₂) δ 146.4, 146.3, 145.2 145.0, 140.7, 136.10, 136.06, 135.8, 133.3, 133.2, 133.1, 132.7, 131.52, 131.49, 131.47, 130.77, 130.75, 127.19, 127.13, 127.0, 123.55, 111.0, 108.2, 71.9, 28.95, 28.90, 28.82, 27.48, 27.32, 27.29, 27.22, 26.5, 22.4, 21.8. (Observed complexity due to C-F and C-P coupling) ppm.

³¹P NMR (162 MHz, CD₂Cl₂) δ 31.72 ppm

¹⁹F NMR (377 MHz, CDCl₃) δ 66.54 ppm.)

FT-IR (Diamond-ATR, neat) \tilde{ν}ₘₐₓ 2973.46 (w), 2923.73 (m), 2853.05 (w), 1455.98 (m), 1400.82 (s), 1204.79 (s), 1112.97 (m).

HRMS calcd for C₃₆H₄₇O₄FPPdS [M-Br]⁺: 731.1946 Da, found: 731.1962 Da.

Synthesis of Peptides with Covalent Warheads

Cys-modified peptides were synthesized as outlined in EXAMPLE 4 and cleaved from the resin using TFA/phenol/H₂O/thioanisole/EDT (82.5:5:5:5:2.5) for 2 h at rt and isolated by three cycles of precipitation with ice-cold diethyl ether and centrifugation. The palladium oxidative addition complex of meta-substituted aryl sulfonyl fluoride electrophilic warhead (2.25 equiv.) was dissolved in MeCN and added to crude peptides (1 equiv.) dissolved in HEPES (0.5 M, pH=7.0) over 30 sec. The solution was mixed thoroughly and allowed to react for 30 min at rt. AcOH was added, and the solution was diluted with H₂O. The peptide was isolated from the reaction mixture by reverse-phase flash chromatography using a Sfar Duo C18 column (12 g). Yield is expressed as % of isolated, pure peptides over crude peptides used for conjugation of the warhead. Exemplary peptide yields and calculated peptide mass are shown in TABLE 12.

TABLE 12

| Peptide | Total Yield | Calculated and LC MS Mass |
|---|---|---|
| VL9-1 | 0.3 mg, 0.2 μmol, | Exact mass: calculated for |
| Cys(mSF) | 4.5% yield | C₅₃H₈₁FN₁₂O₁₃S₃: 1208.52 Da, |
| | | found: 1208.53 Da |
| VL9-2 | 1.2 mg, 0.9 μmol, | Exact mass: calculated for |
| Cys(mSF) | 17% yield | C₅₃H₈₁FN₁₂O₁₃S₂: 1176.55 Da, |
| | | found: 1175.56 Da |
| VL9-3 | 2.0 mg, 1.5 μmol, | Exact mass: calculated for |
| Cys(mSF) | 29% yield | C₅₅H₈₅FN₁₂O₁₃S₃: 1236.55 Da, |
| | | found: 1237.56 Da |
| VL9-4 | 3.5 mg, 2.6 μmol, | Exact mass: calculated for |
| Cys(mSF) | 44% yield | C₅₃H₈₃FN₁₂O₁₃S₃: 1210.54 Da, |
| | | found: 1210.55 Da |
| VL9-5 | 2.6 mg, 2.3 μmol, | Exact mass: calculated for |
| Cys(mSF) | 36% yield | C₅₂H₇₈FN₉O₁₃S₃: 1151.49 Da, |
| | | found: 1151.50 Da |
| VL9-6 | 3.9 mg, 3.0 μmol, | Exact mass: calculated for |
| Cys(mSF) | 42% yield | C₅₄H₈₃FN₁₂O₁₂S₃: 1206.54 Da, |
| | | found: 1206.55 Da |
| VL9-7 | 3.2 mg, 2.4 μmol, | Exact mass: calculated for |
| Cys(mSF) | 41% yield | C₅₂H₇₉FN₁₂O₁₃S₃: 1195.50 Da, |
| | | found: 1195.52 Da |
| VL9-8 | 2.4 mg, 1.9 μmol, | Exact mass: calculated for |
| Cys(mSF) | 28% yield | C₄₉H₈₁FN₁₂O₁₃S₃: 1160.52 Da, |
| | | found: 1161.54 Da |
| VL9-9 | 3.3 mg, 2.5 μmol, | Exact mass: calculated for |
| Cys(mSF) | 14% yield | C₅₂H₇₉FN₁₂O₁₃S₃: 1194.50 Da, |
| | | found: 1194.51 Da |
| B5.1_8 | 4.3 mg, 2.7 μmol, | Exact mass: calculated for |
| Cys(mSF) | 39% yield | C₆₁H₉₆F₂N₁₆O₁₃S₂: 1362.68 Da, |
| | | found: 1362.63 Da |

The Cys peptide variants were equipped with a mSF warhead in a Pd-mediated coupling using Pd oxidative addition complex 1 (FIGS. 6A and 6B) X* indicating position functionalized with the electrophilic warhead). Electrophilic analogs of VL9 were subjected to cross-linking reactions in a 10-fold excess with [HLA-E+VL9] for 4 h at 37° C., and the amount of covalently bound HLA-E was determined by LC-MS as described in EXAMPLE 4. Installing the electrophilic warhead at positions 1, 2, 5, 6, or 9 led to no or only trace amounts of cross-linking with HLA-E, whereas substantial cross-linking was observed with the aryl sulfonyl fluoride at positions 4 (36%), 7 (22%), and in particular at positions 3 (73%) and 8 (87%) (FIG. 6C).

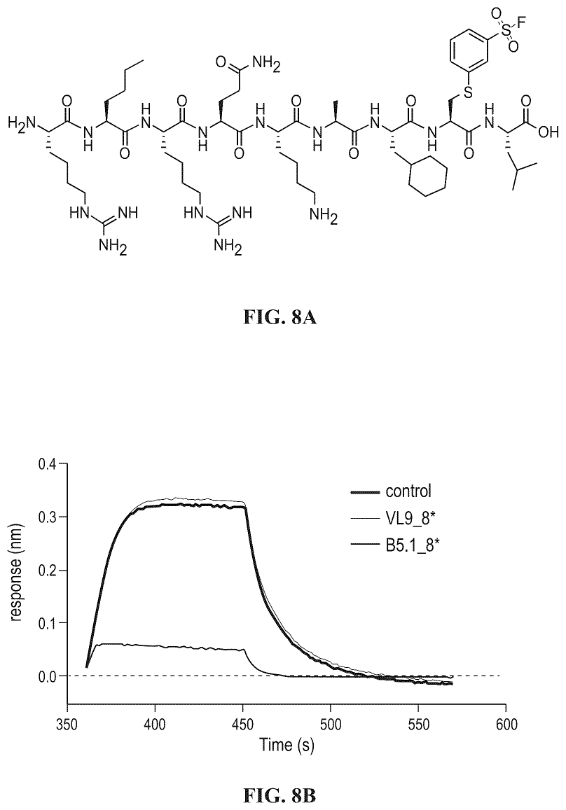

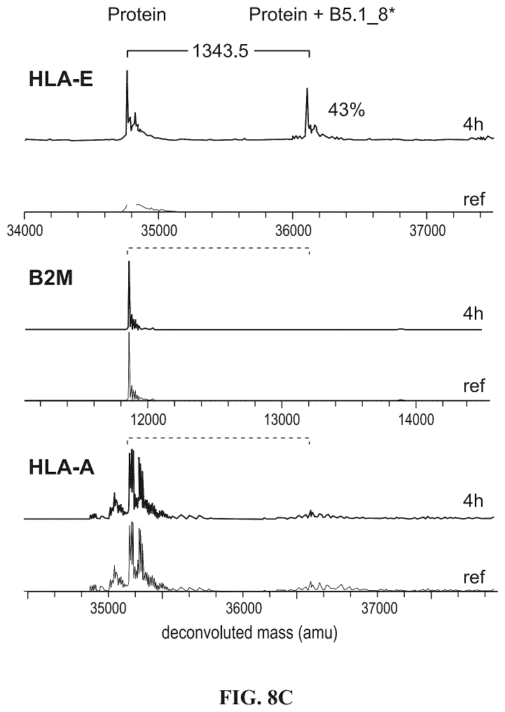

The electrophilic designer peptide B5.1_8* (FIG. 8A) but not electrophilic VL9_8*, derived from the endogenous ligand of HLA-E, was able to reduce the binding of CD94-NKG2A to HLA-E by 85% as was observed by BLI after a 2 h incubation of [HLA-E+VL9] with the electrophilic peptides (FIG. 8B). 43% of cross-linked HLA-E-B5.1_8* could be observed after 4 h of incubation illustrated in FIG. 6B as determined by LC-MS (FIG. 8C). B2M, which is co-expressed with HLA-E for stabilization of the MHC class I molecule and is present at equimolar concentrations in the cross-linking reaction, bears several nucleophilic residues (Lys, Cys, His, Ser, Thr, Tyr). No mass shift was observed for B2M in the cross-linking experiment, indicating that B5.1_8* binds and reacts specifically with HLA-E (FIG. 8C). Cross-linking reactions with HLA-A, another MHC class I molecule, showed no conversion with B5.1_8* within 4 h.

### Example 6—Determination of Inhibition of CD94-NKG2A Binding to HLA-E by Peptides

This example describes the analysis of inhibition of CD94-NKG2A binding to HLA-E by exemplary modified peptides by bio-layer interferometry (BLI).

Additional VL9 based HLA-E binding peptides were designed either based on the library described in EXAMPLE 1.

Further, peptides based on the ligand for the NKG2A/CD94 inhibitory receptor in mice, the nonclassical MHC molecule Qa-1b, the mouse HLA-E ortholog, which presents the peptide AMAPRTLLL, referred to as Qdm (for Qa-1 determinant modifier). This dominant peptide is derived from the leader sequences of murine classical MHC class I encoded by the H-2D) and -L loci.

Briefly, proteins and VL9 or Qdm derived peptides were produced as described in EXAMPLE 2 and EXAMPLE 3. BLI measurements to determine inhibition were performed as described in EXAMPLE 4.

Exemplary modified peptides that were used in the inhibition of CD94-NKG2A binding to HLA-E measured by measurements are shown in TABLES 13, 14, and 15.

TABLE 13

| | | SEQ | |
|---|---|---|---|
| | | ID | BLI |
| Peptide | Amino Acid Sequence | NO: | result |
| B9_1Lys | Lys(TAMRA)-Nle-hAr-Gln-Mff- | 52 | inhibition |
| (TAMRA) | Ala-Cha-hAr-Leu | | |
| B9_5Lys | hAr-Nle-hAr-Gln-Lys(TAMRA)- | 53 | inhibition |
| (TAMRA) | Ala-Cha-hAr-Leu | | |
| B9_8Lys | hAr-Nle-hAr-Gln-Mff-Ala-Cha- | 54 | inhibition |
| (TAMRA) | Lys(TAMRA)-Leu | | |
| B9_LPETGG | hAr-Nle-hAr-Gln-Mff-Ala-Cha- | 55 | inhibition |
| | hAr-Leu-Leu-Pro-Glu-Thr-Gly- | | |
| | Gly | | |
| B9_5Lys | hAr-Nle-hAr-Gln-Lys(DOTA)- | 56 | inhibition |
| (DOTA) | Ala-Cha-hAr-Leu | | |
| B9_5Lys | hAr-Nle-hAr-Gln-Lys(ibu)- | 57 | inhibition |
| (ibu) | Ala-Cha-hAr-Leu | | |
| B9_5Lys | hAr-Nle-hAr-Gln-Lys(FITC)- | 58 | inhibition |
| (FITC) | Ala-Cha-hAr-Leu | | |
| B9_8Lys | hAr-Nle-hAr-Gln-Mff-Ala- | 59 | inhibition |
| (ibu) | Cha-Lys(ibu)-Leu | | |
| B9_5Lys | hAr-Nle-hAr-Gln-Lys(CUM)- | 60 | inhibition |
| (CUM) | Ala-Cha-hAr-Leu | | |
| B4_scrambled | Ala-Cha-Leu-Ala-Met-Arg- | 61 | |
| | 4Af-Ala-Ser | | |
| T1 | Gln-Gln-Phe-DPro-Gly-Asn- | 62 | |
| | Pro-Asn-Gly-Asn-Ala-Leu- | | |
| | Asp-Glu-Ser-CONH2 | | |
| T2 | Gln-Gln-Phe-DPro-Pro-Asn- | 63 | |
| | Pro-Asn-Gly-Asn-Ala-Leu- | | |
| | Asp-Glu-Ser-CONH2 | | |
| T3 | Gln-Gln-Phe-Ala-Aib-Asn- | 64 | |
| | Pro-Asn-Gly-Asn-Ala-Leu- | | |
| | Asp-Glu-Ser-CONH2 | | |
| P3_Nbio | PEG4-Asn-Ala-Leu-Asp-Glu- | 65 | Inhibition |
| | Tyr-Cys-Glu-Asp-Lys-Asn- | | BLOD |
| | Arg-CONH2 | | |
| P3_Cbio | Asn-Ala-Leu-Asp-Glu-Tyr- | 66 | inhibition |
| | Cys-Glu-Asp-Lys-Asn-Arg- | | BLOD |
| | PEG2-Lys(biotin)-CONH2 | | |
| P3_7bio | Asn-Ala-Leu-Asp-Glu-Tyr- | 67 | Inhibition |
| | Lys(PEG4-biotin)-Glu-Asp- | | BLOD |
| | Lys-Asn-Arg-CONH2 | | |
| VL9_2Nle_{—} | Val-Nle-Ala-Pro-Dap(Ser-Ile- | 68 | low |
| 5Dap | Cys(ox.)-His-Gly-Ile-Glu-Tyr- | | inhibition |
| (PhPep5_ox) | Pro-Cys(ox.))-Thr-Leu-Phe-Leu | | |
| VL9_2Nle_{—} | Val-Nle-Ala-Pro-Dap(Ser-Pro- | 69 | low |
| 5Dap | Cys(ox.)-Glu-Arg-Trp-Ala-Ile- | | inhibition |
| (PhPep4_ox) | Ser-Phe-Cys(ox.))-Thr-Leu-Phe- | | |
| | Leu | | |
| VL9_2Nle_{—} | Val-Nle-Ala-Pro-Dap(Ser-His- | 70 | low |
| 5Dap | Cys(ox.)-Ser-Asp-Thr-Gln-Glu- | | inhibition |
| (PhPep3_ox) | Ala-Tyr-Cys(ox.))-Thr-Leu-Phe- | | |
| | Leu | | |
| VL9_2Nle_{—} | Val-Nle-Ala-Pro-Dap(Ser-Trp- | 71 | low |
| 5Dap | Cys(ox.)-Thr-Lys-Thr-Asn-Asp- | | inhibition |
| (PhPep2_ox) | Gln-Arg-Cys(ox.))-Thr-Leu-Phe- | | |
| | Leu | | |
| VL9_2Nle_{—} | Val-Nle-Ala-Pro-Dap(Ser-Phe- | 72 | low |
| 5Dap | Cys(ox.)-Asn-Ile-Ser-Gly-Gly- | | inhibition |
| (PhPep1_ox) | Ile-His-Cys(ox.))-Thr-Leu-Phe-Leu | | |
| VL9_8Cys(mSF) | Val-Met-Ala-Pro-Arg-Thr-Leu- | 128 | inhibition |
| | Cys(mSF)-Leu | | |

BLOD = below the limit of detection

TABLE 14 shows exemplary Qdm derived peptides and BLI results.

TABLE 14

| | | | SEQ |
|---|---|---|---|
| | | | ID |
| Peptide | Amino Acid Sequence | BLI result | NO: |
| P1 | Ala-Met-Cys-Gly-Pro-Arg-Cys-Leu-Trp- | inhibition | 92 |
| | CONH2 | BLOD | |
| P2 | Ala-Leu-Cys-Trp-Arg-Val-Pro-Cys-Thr- | inhibition | 93 |
| | CONH2 | BLOD | |
| P3 | Ala-Met-Cys-Pro-Pro-Ser-Leu-Cys-Phe- | inhibition | 94 |
| | CONH2 | BLOD | |
| P4 | Ala-Met-Cys-Pro-Pro-Trp-Met-Cys-Phe- | inhibition | 95 |
| | CONH2 | BLOD | |
| P5 | Ala-Leu-Cys-Gly-Arg-Ser-Cys-Leu-Val- | inhibition | 96 |
| | CONH2 | BLOD | |
| P6 | Ala-Met-Cys-Gly-Ala-Arg-Cys-Leu-Phe- | inhibition | 97 |
| | CONH2 | BLOD | |
| P7 | Ala-Met-Cys-Gly-Arg-Leu-Cys-Leu-Phe- | inhibition | 98 |
| | CONH2 | BLOD | |
| P8 | Ala-Leu-Cys-Thr-Arg-Val-Pro-Cys-Thr- | inhibition | 99 |
| | CONH2 | BLOD | |
| P9 | Ala-Leu-Cys-Gly-Lys-Ala-Cys-Leu-Tyr- | inhibition | 100 |
| | CONH2 | BLOD | |
| P10 | Ala-Leu-Cys-Gly-Ala-Arg-Cys-Leu-Phe- | inhibition | 101 |
| | CONH2 | BLOD | |
| P11 | Ala-Met-Cys-Trp-Ser-Val-Pro-Cys-Ser- | inhibition | 102 |
| | CONH2 | BLOD | |
| P12 | Ala-Leu-Cys-Pro-Arg-Val-Pro-Cys-Ser- | inhibition | 103 |
| | CONH2 | BLOD | |
| P13 | Ala-Met-Cys-Arg-Gln-Val-Pro-Cys-His- | inhibition | 104 |
| | CONH2 | BLOD | |
| P14 | Ala-Leu-Cys-Ser-Arg-Val-Pro-Cys-Ser- | inhibition | 105 |
| | CONH2 | BLOD | |
| P15 | Ala-Leu-Cys-Gly-Arg-Ala-Cys-Leu-Trp- | inhibition | 106 |
| | CONH2 | BLOD | |

BLOD = below the limit of detection

TABLE 15 shows exemplary VL9 derived peptides with additional amino acids at the N-terminus and BLI results.

TABLE 15

| | | | SEQ |
|---|---|---|---|
| | | | ID |
| Peptide | Amino Acid Sequence | BLI result | NO: |
| G1_B9 | Gly-hAr-Nle-hAr-Gln-Mff-Ala-Cha-hAr- | inhibition | 107 |
| | Leu | | |
| G3_B9 | Gly-Gly-Gly-hAr-Nle-hAr-Gln-Mff-Ala- | inhibition | 108 |
| | Cha-hAr-Leu | BLOD | |
| G5_B9 | Gly-Gly-Gly-Gly-Gly-hAr-Nle-hAr-Gln- | inhibition | 109 |
| | Mff-Ala-Cha-hAr-Leu | BLOD | |

BLOD = below the limit of detection

TABLE 16 shows exemplary VL9 derived peptides.

TABLE 16

| | | SEQ |
|---|---|---|
| Peptide | Amino Acid Sequence | ID NO: |
| B9_1Ala | Ala-Nle-hAr-Gln-Mff-Ala-Cha-hAr-Leu | 73 |
| B9_2Ala | hAr-Ala-hAr-Gln-Mff-Ala-Cha-hAr-Leu | 74 |
| B9_3Ala | hAr-Nle-Ala-Gln-Mff-Ala-Cha-hAr-Leu | 75 |
| B9_4Ala | hAr-Nle-hAr-Ala-Mff-Ala-Cha-hAr-Leu | 76 |
| B9_5Ala | hAr-Nle-hAr-Gln-Ala-Ala-Cha-hAr-Leu | 77 |
| B9_6Gly | hAr-Nle-hAr-Gln-Mff-Gly-Cha-hAr-Leu | 78 |
| B9_7Ala | hAr-Nle-hAr-Gln-Mff-Ala-Ala-hAr-Leu | 79 |
| B9_8Ala | hAr-Nle-hAr-Gln-Mff-Ala-Cha-Ala-Leu | 80 |
| B9_9Ala | hAr-Nle-hAr-Gln-Mff-Ala-Cha-hAr-Ala | 81 |
| B11 | DhAr-Nle-DhAr-Gln-Mff-Ala(NMe)-Cha-DhAr-Leu | 82 |
| B11.1 | DhAr-Nle-hAr-Gln-Mff-Ala(NMe)-Cha-DhAr-Leu | 83 |
| B11.2 | DhAr-Nle-hAr-Gln-Mff-Ala-Cha-hAr-Leu | 84 |
| B11.3 | hAr-Nle-DhAr-Gln-Mff-Ala-Cha-hAr-Leu | 85 |
| B11.4 | hAr-Nle-hAr-Gln-Mff-Ala-Cha-DhAr-Leu | 86 |
| B11.5 | hAr-Nle-hAr-Gln-Mff-Bal-Cha-hAr-Leu | 87 |
| B11.6 | hAr-Nle-hAr-Gln-Mff-Ala(NMe)-Cha-hAr-Leu | 88 |
| B11.7 | hAr-Nle-hAr-Gln-Mff-Ala(NMe)-Cha-DhAr-Leu | 89 |
| B11.8 | DhAr-Nle-hAr-Gln-Mff-Ala-Cha-DhAr-Leu | 90 |
| B11.9 | DhAr-Nle-hAr-Gln-Mff-Ala(NMe)-Cha-hAr-Leu | 91 |

### Example 7—Determination of Covalent Binding of VL9 Modified Analogs to HLA-E

This example describes the analysis of crosslinking of modified VL9 based peptides to HLA-E.

Briefly, crosslinking reactions were performed as described in EXAMPLE 5. Exemplary peptides and crosslinking results are shown in TABLE 17.

TABLE 17

| | | | SEQ |
|---|---|---|---|
| | | Crosslinking | ID |
| Peptide | Amino Acid Sequence | result | NO: |
| Ac-B9_{—} | COCH3-hAr-Nle-hAr-Gln-Mff- | minimal | 110 |
| 8Cys(pSF) | Ala-Cha-Cys(pSF)-Leu | crosslinking | |
| Ac-B9_{—} | COCH3-hAr-Nle-hAr-Gln-Mff- | minimal | 111 |
| 8Cys(pPC) | Ala-Cha-Cys(pPC)-Leu | crosslinking | |
| Ac-B9_{—} | COCH3-hAr-Nle-hAr-Gln-Mff- | minimal | 112 |
| 8Lys(Tz-SQ) | Ala-Cha-Lys(Tz-SQ)-Leu | crosslinking | |
| Ac-B9_{—} | COCH3-hAr-Nle-hAr-Gln-Mff- | low cross- | 113 |
| 8Cys(SF) | Ala-Cha-Cys(mSF)-Leu | linking | |
| VL9_{—} | Cys(mSF)-Met-Ala-Pro-Arg- | no cross- | 114 |
| 1Cys(mSF) | Thr-Leu-Phe-Leu | linking | |
| VL9_{—} | Val-Cys(mSF)-Ala-Pro-Arg- | no cross- | 115 |
| 2Cys(mSF) | Thr-Leu-Phe-Leu | linking | |
| VL9_{—} | Val-Met-Cys(mSF)-Pro-Arg- | potent | 116 |
| 3Cys(mSF) | Thr-Leu-Phe-Leu | crosslinking | |
| VL9_{—} | Val-Met-Ala-Cys(mSF)-Arg- | moderate | 117 |
| 4Cys(mSF) | Thr-Leu-Phe-Leu | crosslinking | |
| VL9_{—} | Val-Met-Ala-Pro-Cys(mSF)- | low cross- | 118 |
| 5Cys(mSF) | Thr-Leu-Phe-Leu | linking | |
| VL9_{—} | Val-Met-Ala-Pro-Arg- | minimal | 119 |
| 6Cys(mSF) | Cys(mSF)-Leu-Phe-Leu | crosslinking | |
| VL9_{—} | Val-Met-Ala-Pro-Arg-Thr- | low cross- | 120 |
| 7Cys(mSF) | Cys(mSF)-Phe-Leu | linking | |
| VL9_{—} | Val-Met-Ala-Pro-Arg-Thr- | minimal | 121 |
| 9Cys(mSF) | Leu-Phe-Cys(mSF) | crosslinking | |
| VL9_3 | Val-Met-Cys(mSF)-Pro-Arg- | potent | 122 |
| Cys(mSF)_{—} | Thr-Leu-Cys(mSF)-Leu | crosslinking | |
| 8Cys(mSF) | | | |
| B9_3Lys(Tz- | hAr-Nle-Lys(Tz-SQ)-Gln-Mff- | no cross- | 123 |
| SQ) | Ala-Cha-hAr-Leu | linking | |
| B9_3Lys(Tz- | hAr-Nle-Lys(Tz-PTAD)-Gln- | no cross- | 124 |
| PTAD) | Mff-Ala-Cha-hAr-Leu | linking | |
| B9_8Lys(Tz- | hAr-Nle-hAr-Gln-Mff-Ala- | no cross- | 125 |
| SQ) | Cha-Lys(Tz-SQ)-Leu | linking | |
| B9_8Lys(Tz- | hAr-Nle-hAr-Gln-Mff-Ala- | no cross- | 126 |
| PTAD) | Cha-Lys(Tz-PTAD)-Leu | linking | |
| B9_scrambled_{—} | Cha-hAr-Ala-Leu-hAr-Gln- | no cross- | 127 |
| 8Cys(mSF) | Nle-Cys(mSF)-hAr | linking | |
| VL9_8Dha | Val-Met-Ala-Pro-Arg-Thr- | no cross- | 128 |
| | Leu-Dha-Leu | linking | |
| VL9_{—} | Val-Met-Ala-Pro-Arg-Thr- | potent cross- | 129 |
| 8Cys(mSF) | Leu-Cys(mSF)-Leu | linking, BLI | |
| | | inhibition | |
| Ac-B9_{—} | COCH3-Cys(mSF)-Nle-hAr- | low cross- | 130 |
| 1Cys(mSF) | Gln-Mff-Ala-Cha-hAr-Leu | linking | |
| Ac- | COCH3-hAr-Cys(mSF)-hAr- | low cross- | 131 |
| B9_2Cys(mSF) | Gln-Mff-Ala-Cha-hAr-Leu | linking | |
| Ac-B9_{—} | COCH3-Dap(Tz-PTAD)-Nle- | no cross- | 132 |
| 1Dap(Tz-PTAD) | hAr-Gln-Mff-Ala-Cha-hAr-Leu | linking | |
| Ac-B9_{—} | COCH3-hAr-Dap(Tz-PTAD)- | no cross- | 133 |
| 2Dap(Tz-PTAD) | hAr-Gln-Mff-Ala-Cha-hAr-Leu | linking | |
| B9_8Cys(pSF) | hAr-Nle-hAr-Gln-Mff-Ala- | moderate | 134 |
| | Cha-Cys(pSF)-Leu | crosslinking | |
| B9_8Cys(mSF) | hAr-Nle-hAr-Gln-Mff-Ala- | potent | 135 |
| | Cha-Cys(mSF)-Leu | crosslinking | |
| B9_8Cys(pPC) | hAr-Nle-hAr-Gln-Mff-Ala- | low cross- | 136 |
| | Cha-Cys(pPC)-Leu | linking | |
| B9_{—} | hAr-Nle-hAr-Gln-Lys(PEG2- | no cross- | 137 |
| 5Lys(FITC)_{—} | FITC)-Ala-Cha-Cys-Leu | linking | |
| 8Cys | | | |
| B9_scrambled_{—} | Cha-Cys(mSF)-Ala-Leu-hAr- | minimal | 138 |
| 2Cys(mSF) | Gln-Nle-Mff-hAr | crosslinking | |
| B9_{—} | hAr-Nle-Cys(mSF)-Gln-Mff- | minimal | 139 |
| 3Cys(mSF) | Ala-Cha-hAr-Leu | crosslinking | |
| VL9_{—} | Val-Met-Ala-Pro-Lys(PEG2- | potent | 140 |
| 5Lys(DOTA)_{—} | DOTA)-Thr-Leu- | crosslinking | |
| 8Cys(mSF) | Cys(mSF)-Leu | | |
| VL9_{—} | Val-Met-Ala-Pro-Lys(PEG2- | moderate | 141 |
| 5Lys(FITC)_{—} | FITC)-Thr-Leu-Cys(mSF)-Leu | crosslinking | |
| 8Cys(mSF) | | | |
| VL9_{—} | Val-Met-Cys(mSF)-Pro- | moderate | 142 |
| 3Cys(mSF)_{—} | Lys(DOTA)-Thr-Leu-Phe-Leu | crosslinking | |
| 5Lys(DOTA) | | | |
| VL9_{—} | Val-Met-Cys(mSF)-Pro- | potent | 143 |
| 3Cys(mSF)_{—} | Lys(PEG4-biotin)-Thr- | crosslinking | |
| 5Lys(bio) | Leu-Phe-Leu | | |
| VL9_{—} | Val-Met-Cys(mSF)-Pro- | moderate | 144 |
| 3Cys(mSF)_{—} | Lys(PEG2-ibu)-Thr-Leu- | crosslinking | |
| 5Lys(ibu) | Phe-Leu | | |
| VL9_{—} | Val-Met-Ala-Pro-Lys(PEG2- | moderate | 145 |
| 5Lys(ibu)_{—} | ibu)-Thr-Leu-Cys(mSF)-Leu | crosslinking | |
| 8Cys(mSF) | | | |
| VL9_{—} | Val-Met-Ala-Pro-Lys(PEG4- | potent | 146 |
| 5Lys(biotin)_{—} | biotin)-Thr-Leu-Cys(mSF)-Leu | crosslinking | |
| 8Cys(mSF) | | | |
| VL9_{—} | Val-Met-Ala-Pro-Lys(Tz- | moderate | 147 |
| 5Lys(Tz- | CD94[111-114])-Thr-Leu- | crosslinking | |
| CD94[111- | Cys(mSF)-Leu | | |
| 114])_{—} | | | |
| 8Cys(mSF) | | | |
| VL9_{—} | Val-Met-Lys(Tz-NKG2A[213- | low cross- | 148 |
| 3Lys(Tz- | 217])-Pro-Arg-Thr-Leu- | linking | |
| NKG2A[213- | Cys(mSF)-Leu | | |
| 217])_{—} | | | |
| 8Cys(mSF) | | | |
| VL9_{—} | Val-Met-Ala-Pro-Arg-Thr- | low cross- | 149 |
| 8Cys(pPC) | Leu-Cys(pPC)-Leu | linking | |
| VL9_{—} | Val-Met-Ala-Pro-Arg-Thr- | low cross- | 150 |
| 8Cys(pSF) | Leu-Cys(pSF)-Leu | linking | |
| VL9_{—} | Val-Met-Ala-Pro-Arg-Thr- | no cross- | 151 |
| 8Cys(NHS) | Leu-Cys(NHS)-Leu | linking | |
| VL9_{—} | Val-Met-Ala-Pro-Arg-Thr- | minimal | 152 |
| 8Cys(mPC) | Leu-Cys(mPC)-Leu | crosslinking | |
| VL9_{—} | Val-Met-Ala-Pro-Arg-Thr- | minimal | 153 |
| 8Cys(mSN) | Leu-Cys(mSN)-Leu | crosslinking | |
| VL9_{—} | Val-Met-Ala-Pro-Arg-Thr- | no cross- | 154 |
| 8Cys(pSN) | Leu-Cys(pSN)-Leu | linking | |
| VL9_1DMBA_{—} | DMBA-Met-Ala-Pro-Arg-Thr- | low cross- | 155 |
| 8Cys(mSF) | Leu-Cys(mSF)-Leu | linking | |
| VL9_1HMPA_{—} | HMPA-Met-Ala-Pro-Arg-Thr- | low cross- | 156 |
| 8Cys(mSF) | Leu-Cys(mSF)-Leu | linking | |
| VL9_{—} | NMeH-Val-Met-Ala-Pro-Arg- | low cross- | 157 |
| 1Val(NMe)_{—} | Thr-Leu-Cys(mSF)-Leu | linking | |
| 8Cys(mSF) | | | |
| VL9_{—} | Val-Met-Ala-Pro-Arg-Thr- | low cross- | 158 |
| 8Dab(3SF) | Leu-Dab(3SF)-Leu | linking | |
| VL9_{—} | Val-Met-Ala-Pro-Arg-Thr- | low cross- | 159 |
| 8Dab(4SF) | Leu-Dab(4SF)-Leu | linking | |
| VL9_{—} | Val-Met-Ala-Pro-Arg-Thr- | low cross- | 160 |
| 8Dab(3SF4OMe) | Leu-Dab(3SF4OMe)-Leu | linking | |
| VL9_{—} | Val-Met-Ala-Pro-Arg-Thr- | low cross- | 161 |
| 8Dab(5SF2OMe) | Leu-Dab(5SF2OMe)-Leu | linking | |
| VL9_{—} | Val-Met-Ala-Pro-Arg-Thr- | low cross- | 162 |
| 8Dap(3SF) | Leu-Dap(3SF)-Leu | linking | |
| VL9_{—} | Val-Met-Ala-Pro-Arg-Thr- | low cross- | 163 |
| 8Dap(4SF) | Leu-Dap(4SF)-Leu | linking | |
| VL9_{—} | Val-Met-Ala-Pro-Arg-Thr- | low cross- | 164 |
| 8Dap(3SF4OMe) | Leu-Dap(3SF4OMe)-Leu | linking | |
| VL9_{—} | Val-Met-Ala-Pro-Arg-Thr- | low cross- | 165 |
| 8Dap(5SF2OMe) | Leu-Dap(5SF2OMe)-Leu | linking | |
| VL9_1HMBA_{—} | HMBA-Met-Ala-Pro-Arg-Thr- | moderate | 166 |
| 8Cys(mSF) | Leu-Cys(mSF)-Leu | crosslinking | |
| VL9_{—} | Val-Met-Ala-Pro-Arg-Thr- | | 167 |
| 8Cys(mFSF) | Leu-Cys(mFSF)-Leu | | |
| VL9_{—} | Val-Met-Ala-Pro-Arg-Thr- | | 168 |
| 8Cys(mMeSF) | Leu-Cys(mMeSF)-Leu | | |
| VL9_{—} | Val-Met-Ala-Pro-Arg-Thr- | | 169 |
| 8Cys(mPySF) | Leu-Cys(mPySF)-Leu | | |
| VL9_{—} | Val-Met-Ala-Pro-Arg-Thr- | no cross- | 170 |
| 8Cys(SAL) | Leu-Cys(SAL)-Leu | linking | |
| VL9_2Nle_{—} | Val-Nle-Ala-Pro-Arg-Thr- | | 171 |
| 8Cys(mSF) | Leu-Cys(mSF)-Leu | | |
| VL9_2Nle_{—} | Val-Nle-Ala-Pro-Arg-Thr- | | 172 |
| 8hCys(mSF) | Leu-hCys(mSF)-Leu | | |
| VL9_2Nle_{—} | Val-Nle-Ala-Pro-Pgl-Thr- | | 173 |
| 5Pgl_8Cys | Leu-Cys(mSF)-Leu | | |
| VL9_2Nle_{—} | Val-Nle-Ala-Pro-Bhp-Thr- | | 174 |
| 5Bhp_{—} | Leu-Cys(mSF)-Leu | | |
| 8Cys(mSF) | | | |
| VL9_5Ala_{—} | Val-Met-Ala-Pro-Ala-Thr- | | 175 |
| 8(mSF) | Leu-Cys(mSF)-Leu | | |
| VL9_5Lys_{—} | Val-Met-Ala-Pro-Lys-Thr- | | 176 |
| 8(mSF) | Leu-Cys(mSF)-Leu | | |
| VL9_{—} | Val-Met-Ala-Pro-DArg-Thr- | no cross- | 177 |
| 8Cys(mACA) | Leu-Cys(mACA)-Leu | linking | |
| VL9_{—} | Val-Met-Ala-Pro-DArg-Thr- | no cross- | 178 |
| 8Cys(mSAC) | Leu-Cys(mSAC)-Leu | linking | |
| VL9_{—} | Val-Met-Ala-Pro-Arg-Thr- | no cross- | 179 |
| 8Cys(mAld) | Leu-Cys(mAld)-Leu | linking | |
| VL9_{—} | Val-Met-Ala-Pro-Arg-Thr- | no cross- | 180 |
| 8Cys(oAld) | Leu-Cys(oAld)-Leu | linking | |
| VL9_5hArg_{—} | Val-Met-Ala-Pro-hArg-Thr- | no cross- | 181 |
| 8Cys(mSF) | Leu-Cys(mSF)-Ala | linking | |
| VL9_5Cav_{—} | Val-Met-Ala-Pro-Cav-Thr- | no cross- | 182 |
| 8Cys(mSF) | Leu-Cys(mSF)-Ala | linking | |
| VL9_5DArg_{—} | Val-Met-Ala-Pro-DArg-Thr- | no cross- | 183 |
| 8Cys(mSF) | Leu-Cys(mSF)-Ala | linking | |
| VL9R_9Ala_{—} | Val-Met-Ala-Pro-Arg-Thr- | | 184 |
| 8Cys(mSF) | Leu-Cys(mSF)-Ala | | |
| VL9R_7Ala_{—} | Val-Met-Ala-Pro-Arg-Thr- | | 185 |
| 8Cys(mSF) | Ala-Cys(mSF)-Leu | | |
| VL9R_6Ala_{—} | Val-Met-Ala-Pro-Arg-Ala- | | 186 |
| 8Cys(mSF) | Leu-Cys(mSF)-Leu | | |
| VL9R_5Ala_{—} | Val-Met-Ala-Pro-Ala-Thr- | | 187 |
| 8Cys(mSF) | Leu-Cys(mSF)-Leu | | |
| VL9R_4Ala_{—} | Val-Met-Ala-Ala-Arg-Thr- | | 188 |
| 8Cys(mSF) | Leu-Cys(mSF)-Leu | | |
| VL9R_2Ala_{—} | Val-Ala-Ala-Pro-Arg-Thr- | | 189 |
| 8Cys(mSF) | Leu-Cys(mSF)-Leu | | |
| VL9R_1Ala_{—} | Ala-Met-Ala-Pro-Arg-Thr- | | 190 |
| 8Cys(mSF) | Leu-Cys(mSF)-Leu | | |
| VL9R_3Gly_{—} | Val-Met-Gly-Pro-Arg-Thr- | | 191 |
| 8Cys(mSF) | Leu-Cys(mSF)-Leu | | |
| B11.1_{—} | dhAr-Nle-lhAr-Gln-Mff- | | 192 |
| 8Cys(mSF) | Ala(N-Methyl)-Cha-Cys(mSF)- | | |
| | Leu | | |
| B11.8_{—} | dhAr-Nle-lhAr-Gln-Mff- | | 193 |
| 8Cys(mSF) | Ala-Cha-Cys(mSF)-Leu | | |

### Example 8—Determination of Binding of VL9 Modified Analogs to HLA-E by Fluorescence Polarization

This example describes the analysis of binding of modified VL9 based peptides to HLA-E by Fluorescence Polarization (FP).

Briefly, proteins and VL9 or Qdm derived peptides were produced as described in EXAMPLE 2 and EXAMPLE 3 and binding to HLA-E was measured with Fluorescence Polarization.

Exemplary peptides and FP results are shown in TABLE 18.

TABLE 18

| | | | SEQ |
|---|---|---|---|
| | | FP | ID |
| Peptide | Amino Acid Sequence | result | NO: |
| VL9_{—} | Val-Met-Ala-Pro-Lys(FITC)-Thr-Leu- | Exchange | 194 |
| 5Lys(FITC) | Phe-Leu | | |
| P1(FITC) | Ala-Met-Cys-Gly-Pro-Arg-Cys-Leu- | Binding | 195 |
| | Trp-Gly-Gly-Lys(FITC) | | |
| P3(FITC) | Ala-Met-Cys-Pro-Pro-Ser-Leu-Cys- | No | 196 |
| | Phe-Gly-Gly-Lys(FITC) | binding | |
| P5(FITC) | Ala-Leu-Cys-Gly-Arg-Ser-Cys-Leu- | No | 197 |
| | Val-Gly-Gly-Lys(FITC) | binding | |
| P7(FITC) | Ala-Met-Cys-Gly-Arg-Leu-Cys-Leu- | No | 198 |
| | Phe-Gly-Gly-Lys(FITC) | binding | |
| P8(FITC) | Ala-Leu-Cys-Thr-Arg-Val-Pro-Cys- | No | 199 |
| | Thr-Gly-Gly-Lys(FITC) | binding | |
| P9(FITC) | Ala-Leu-Cys-Gly-Lys-Ala-Cys-Leu- | No | 200 |
| | Tyr-Gly-Gly-Lys(FITC) | binding | |
| P10(FITC) | Ala-Leu-Cys-Gly-Ala-Arg-Cys-Leu- | No | 201 |
| | Phe-Gly-Gly-Lys(FITC) | binding | |
| P11(FITC) | Ala-Met-Cys-Trp-Ser-Val-Pro-Cys- | No | 202 |
| | Ser-Gly-Gly-Lys(FITC) | binding | |
| P12(FITC) | Ala-Leu-Cys-Pro-Arg-Val-Pro-Cys- | No | 203 |
| | Ser-Gly-Gly-Lys(FITC) | binding | |
| P13(FITC) | Ala-Met-Cys-Arg-Gln-Val-Pro-Cys- | No | 204 |
| | His-Gly-Gly-Lys(FITC) | binding | |
| P14(FITC) | Ala-Leu-Cys-Ser-Arg-Val-Pro-Cys- | No | 205 |
| | Ser-Gly-Gly-Lys(FITC) | binding | |
| P15(FITC) | Ala-Leu-Cys-Gly-Arg-Ala-Cys-Leu- | No | 206 |
| | Trp-Gly-Gly-Lys(FITC) | binding | |
| P1.2 | Ala-Met-Cys-Gly-Pro-Arg-Cys-Leu- | No | 207 |
| | Trp-Leu-Gly-Gly-Lys(FITC) | binding | |
| P1.3 | Ala-Met-Cys-Gly-Pro-Arg-Cys-Leu- | No | 208 |
| | Phe-Leu-Gly-Gly-Lys(FITC) | binding | |
| P1.4 | Ala-Met-Cys-Gly-Pro-Arg-Cys-Leu- | No | 209 |
| | Gly-Gly-Lys(FITC) | binding | |
| P16(FITC) | Ala-Met-Cys-Asn-Arg-Val-Pro-Cys- | No | 210 |
| | Thr-Gly-Gly-Lys(FITC) | binding | |
| P17(FITC) | Ala-Met-Cys-Pro-Thr-Ile-Pro-Cys- | No | 211 |
| | Ser-Gly-Gly-Lys(FITC) | binding | |
| P19(FITC) | Ala-Leu-Cys-Trp-Arg-Val-Arg-Cys- | No | 212 |
| | Thr-Gly-Gly-Lys(FITC) | binding | |
| P20(FITC) | Ala-Leu-Cys-Trp-Arg-Val-Pro-Cys- | Binding | 213 |
| | Thr-Gly-Gly-Lys(FITC) | | |
| P21(FITC) | Ala-Leu-Cys-Ala-Pro-Arg-Leu-Cys- | Binding | 214 |
| | Leu-Gly-Gly-Lys(FITC) | | |
| P22(FITC) | Ala-Leu-Cys-Asp-Pro-Ser-Lys-Cys- | No | 215 |
| | Phe-Gly-Gly-Lys(FITC) | binding | |
| P23(FITC) | Ala-Leu-Cys-Trp-His-Val-Pro-Cys- | Binding | 216 |
| | Gln-Gly-Gly-Lys(FITC) | | |
| P24(FITC) | Ala-Leu-Cys-Asp-Pro-Arg-Trp-Cys- | No | 217 |
| | Phe-Gly-Gly-Lys(FITC) | binding | |
| P25(FITC) | Ala-Met-Cys-Gly-Lys-Asn-Cys-Leu- | No | 218 |
| | Phe-Gly-Gly-Lys(FITC) | binding | |
| P26(FITC) | Ala-Leu-Cys-Gly-Arg-Pro-Cys-Leu- | No | 219 |
| | Tyr-Gly-Gly-Lys(FITC) | binding | |
| P27(FITC) | Ala-Met-Cys-Gly-Lys-Tyr-Cys-Leu- | No | 220 |
| | Phe-Gly-Gly-Lys(FITC) | binding | |
| P28(FITC) | Ala-Leu-Cys-Arg-Trp-Arg-Cys-Leu- | Binding | 221 |
| | Tyr-Gly-Gly-Lys(FITC) | | |
| P29(FITC) | Ala-Leu-Cys-Gly-Trp-Arg-Cys-Leu- | No | 222 |
| | Tyr-Gly-Gly-Lys(FITC) | binding | |
| P30(FITC) | Ala-Met-Cys-Val-Pro-Arg-Cys-Leu- | No | 223 |
| | Trp-Gly-Gly-Lys(FITC) | binding | |
| P31(FITC) | Ala-Leu-Cys-Gly-Ile-Ala-Cys-Phe- | No | 224 |
| | Pro-Gly-Gly-Lys(FITC) | binding | |
| P32(FITC) | Ala-Met-Cys-Gly-Ala-Leu-Cys-Leu- | Binding | 225 |
| | Phe-Gly-Gly-Lys(FITC) | | |
| P20 | Ala-Leu-Cys-Trp-Arg-Val-Pro-Cys- | Binding | 226 |
| | Thr | | |
| P21 | Ala-Leu-Cys-Ala-Pro-Arg-Leu-Cys-Leu | Binding | 227 |
| P24 | Ala-Leu-Cys-Asp-Pro-Arg-Trp-Cys-Phe | No | 228 |
| | | binding | |
| P29 | Ala-Leu-Cys-Gly-Trp-Arg-Cys-Leu-Tyr | No | 229 |
| | | binding | |
| P32 | Ala-Met-Cys-Gly-Ala-Leu-Cys-Leu-Phe | Binding | 230 |

### Example 9—Determination of Binding of VL9 Modified Analogs to HLA-E

This example describes the analysis of binding of peptides to HLA-E by Fluorescence Polarization (FP), BLI or crosslinking.

Briefly, proteins and VL9 or Qdm derived peptides are produced as described in EXAMPLE 2 and EXAMPLE 3 and binding to HLA-E is measured with Fluorescence Polarization, BLI, or crosslinking as described in EXAMPLE 4, 5, or 7.

Exemplary peptides that can be analyzed are shown in TABLE 19.

TABLE 19

| | | SEQ |
|---|---|---|
| Peptide | Amino Acid Sequence | ID NO: |
| Ac-B9_8Cys | COCH3-hAr-Nle-hAr-Gln-Mff-Ala-Cha-Cys-Leu-COOH | 231 |
| B9_1Lys(biotin) | NH2-Lys(biotin)-Nle-hAr-Gln-Mff-Ala-Cha-hAr-Leu-COOH | 232 |
| B9_5Lys(biotin) | NH2-hAr-Nle-hAr-Gln-Lys(biotin)-Ala-Cha-hAr-Leu-COOH | 233 |
| B9_8Lys(biotin) | NH2-hAr-Nle-hAr-Gln-Mff-Ala-Cha-Lys(biotin)-Leu-COOH | 234 |
| Ac-B9_1Cys | COCH3-Cys-Nle-hAr-Gln-Mff-Ala-Cha-hAr-Leu-COOH | 235 |
| Ac-B9_2Cys | COCH3-hAr-Cys-hAr-Gln-Mff-Ala-Cha-hAr-Leu-COOH | 236 |
| Ac-B9_1Dap(N3) | COCH3-Dap(N3)-Nle-hAr-Gln-Mff-Ala-Cha-hAr-Leu-COOH | 237 |
| Ac-B9_2Dap(N3) | COCH3-hAr-Dap(N3)-hAr-Gln-Mff-Ala-Cha-hAr-Leu-COOH | 238 |
| B9_5Lys(DOTA)_8Lys(ibu) | hAr-Nle-hAr-Gln-Lys(DOTA)-Ala-Cha-Lys(ibu)-Leu-COOH | 239 |
| B9_1Cys | NH2-Cys-Nle-hAr-Gln-Mff-Ala-Cha-hAr-Leu-COOH | 240 |
| B9_8Cys | NH2-hAr-Nle-hAr-Gln-Mff-Ala-Cha-Cys-Leu-COOH | 241 |
| VL9_1Cys | NH2-Cys-Met-Ala-Pro-Arg-Thr-Leu-Phe-Leu-COOH | 242 |
| VL9_2Cys | NH2-Val-Cys-Ala-Pro-Arg-Thr-Leu-Phe-Leu-COOH | 243 |
| VL9_3Cys | NH2-Val-Met-Cys-Pro-Arg-Thr-Leu-Phe-Leu-COOH | 244 |
| VL9_4Cys | NH2-Val-Met-Ala-Cys-Arg-Thr-Leu-Phe-Leu-COOH | 245 |
| VL9_5Cys | NH2-Val-Met-Ala-Pro-Cys-Thr-Leu-Phe-Leu-COOH | 246 |
| VL9_6Cys | NH2-Val-Met-Ala-Pro-Arg-Cys-Leu-Phe-Leu-COOH | 247 |
| VL9_7Cys | NH2-Val-Met-Ala-Pro-Arg-Thr-Cys-Phe-Leu-COOH | 248 |
| VL9_8Cys | NH2-Val-Met-Ala-Pro-Arg-Thr-Leu-Cys-Leu-COOH | 249 |
| VL9_9Cys | NH2-Val-Met-Ala-Pro-Arg-Thr-Leu-Phe-Cys-COOH | 250 |
| VL9_3Cys_8Cys | NH2-Val-Met-Cys-Pro-Arg-Thr-Leu-Cys-Leu-COOH | 251 |
| B9_3Lys(N3) | NH2-hAr-Nle-Lys(N3)-Gln-Mff-Ala-Cha-hAr-Leu-COOH | 252 |
| B9_8Lys(N3) | NH2-hAr-Nle-hAr-Gln-Mff-Ala-Cha-Lys(N3)-Leu-COOH | 253 |
| B9_scrambled_8Cys | NH2-Cha-hAr-Ala-Leu-hAr-Gln-Nle-Cys-hAr-COOH | 254 |
| P3_UF | NH2-Asn-Ala-Leu-Asp-Glu-Tyr-Cys-Glu-Asp-Lys-Asn-Arg-CONH2 | 255 |
| P3_7FITC | NH2-Asn-Ala-Leu-Asp-Glu-Tyr-Lys(PEG2-FITC)-Glu-Asp-Lys-Asn-Arg-CONH2 | 256 |
| B9_scrambled_2Cys | NH2-Cha-Cys-Ala-Leu-hAr-Gln-Nle-Mff-hAr-COOH | 257 |
| B9_3Cys | NH2-hAr-Nle-Cys-Gln-Mff-Ala-Cha-hAr-Leu-COOH | 258 |
| VL9_5Lys(DOTA)_8Cys | NH2-Val-Met-Ala-Pro-Lys(PEG2-DOTA)-Thr-Leu-Cys-Leu-COOH | 259 |
| VL9_5Lys(FITC)_8Cys | NH2-Val-Met-Ala-Pro-Lys(PEG2-FITC)-Thr-Leu-Cys-Leu-COOH | 260 |
| VL9_3Cys_5Lys(FITC) | NH2-Val-Met-Cys-Pro-Lys(FITC)-Thr-Leu-Phe-Leu-COOH | 261 |
| VL9_3Cys_5Lys(DOTA) | NH2-Val-Met-Cys-Pro-Lys(DOTA)-Thr-Leu-Phe-Leu-COOH | 262 |
| VL9_3Cys_5Lys(biotin) | NH2-Val-Met-Cys-Pro-Lys(PEG4-biotin)-Thr-Leu-Phe-Leu-COOH | 263 |
| VL9_3Cys_5Lys(ibu) | NH2-Val-Met-Cys-Pro-Lys(PEG2-ibu)-Thr-Leu-Phe-Leu-COOH | 264 |
| VL9_5Lys(ibu)_8Cys | NH2-Val-Met-Ala-Pro-Lys(PEG2-ibu)-Thr-Leu-Cys-Leu-COOH | 265 |
| VL9_5Lys(biotin)_8Cys | NH2-Val-Met-Ala-Pro-Lys(PEG4-biotin)-Thr-Leu-Cys-Leu-COOH | 266 |
| B9_4Gln(13C5,15N2) | NH2-hAr-Nle-hAr-Gln(13C5, 15N2)-Mff-Ala-Cha-hAr-Leu-COOH | 267 |
| VL9_3Cys(mSF)_5Lys(FITC) | NH2-Val-Met-Cys(mSF)-Pro-Lys(FITC)-Thr-Leu-Phe-Leu-COOH | 268 |
| VL9_5Lys(LYTAC)_8Cys | NH2-Val-Met-Ala-Pro-Lys(PEG5-Lys(GalNAc)-Lys(GalNAc)-Lys(GalNAc))-Thr-Leu-Cys- | 269 |
| | Leu-COOH | |
| NKG2A[213-217]_1Bhp | NH2-Bhp-Val-Asn-Arg-Leu-Lys-COOH | 270 |
| CD94[111-114]_1Bhp | NH2-Bhp-Ser-Gln-Gln-Phe-COOH | 271 |
| VL9_5Lys(Tz-CD94 [111-114])_8Cys | NH2-Val-Met-Ala-Pro-Lys(Tz-CD94[111-114])-Thr-Leu-Cys-Leu-COOH | 272 |
| VL9_3Lys(Tz-NKG2A[213-217])_8Cys | NH2-Val-Met-Lys(Tz-NKG2A[213-217])-Pro-Arg-Thr-Leu-Cys-Leu-COOH | 273 |
| VL9_5Bhp_8Cys | NH2-Val-Met-Ala-Pro-Bhp-Thr-Leu-Cys-Leu-COOH | 274 |
| NKG2A[123-232]_130Ser_163Nle_{—} | NH2-NKG2A[123-232]_130Ser_163Nle_171Pro(N3)_189Nle; UniProt P26715-CONH2 | 275 |
| 171Pro(N3)_189Nle | | |
| VL9_5Bhp(Tz-NKG2A[123-232]_{—} | NH2-Val-Met-Ala-Pro-Bhp(Tz-NKG2A[123-232]_130Ser_163Nle_171Pro(Tz)_189Nle)- | 276 |
| 130Ser_163Nle_171Pro(Tz)_189Nle_8Cys | Thr-Leu-Cys-Leu-COOH | |
| VL9_5Bhp(Tz-NKG2A [123-232]_{—} | NH2-Val-Met-Ala-Pro-Bhp(Tz-NKG2A[123-232]_130Ser_163Nle_171Pro(Tz)_189Nle)- | 277 |
| 130Ser_163Nle_171Pro(Tz)_189Nle_8Cys | Thr-Leu-Cys-Leu-COOH | |
| PhPep1 | NH2-Ser-Phe-Cys-Asn-Ile-Ser-Gly-Gly-Ile-His-Cys-CONH2 | 278 |
| PhPep2 | NH2-Ser-Trp-Cys-Thr-Lys-Thr-Asn-Asp-Gln-Arg-Cys-CONH2 | 279 |
| PhPep3 | NH2-Ser-His-Cys-Ser-Asp-Thr-Gln-Glu-Ala-Tyr-Cys-CONH2 | 280 |
| PhPep4 | NH2-Ser-Pro-Cys-Glu-Arg-Trp-Ala-Ile-Ser-Phe-Cys-CONH2 | 281 |
| PhPep5 | NH2-Ser-Ile-Cys-His-Gly-Ile-Glu-Tyr-Pro-Cys-CONH2 | 282 |
| VL9_2Nle_5Dap(Hya) | NH2-Val-Nle-Ala-Pro-Dap(Hya)-Thr-Leu-Phe-Leu-COOH | 283 |
| PhPep1_ox | NH2-Ser-Phe-Cys(ox.)-Asn-Ile-Ser-Gly-Gly-Ile-His-Cys(ox.)-CONH2 | 284 |
| PhPep2_ox | NH2-Ser-Trp-Cys(ox.)-Thr-Lys-Thr-Asn-Asp-Gln-Arg-Cys(ox.)-CONH2 | 285 |
| PhPep3_ox | NH2-Ser-His-Cys(ox.)-Ser-Asp-Thr-Gln-Glu-Ala-Tyr-Cys(ox.)-CONH2 | 286 |
| PhPep4_ox | NH2-Ser-Pro-Cys(ox.)-Glu-Arg-Trp-Ala-Ile-Ser-Phe-Cys(ox.)-CONH2 | 287 |
| PhPep5_ox | NH2-Ser-Ile-Cys(ox.)-His-Gly-Ile-Glu-Tyr-Pro-Cys(ox.)-CONH2 | 288 |
| VL9_scrambled_2Cys | NH2-Leu-Cys-Thr-Leu-Val-Pro-Met-Ala-Arg-COOH | 289 |
| VL9_scrambled | NH2-Leu-Phe-Thr-Leu-Val-Pro-Met-Ala-Arg-COOH | 290 |
| VL9_scrambled_2Cys(mSF) | NH2-Leu-Cys(mSF)-Thr-Leu-Val-Pro-Met-Ala-Arg-COOH | 291 |
| VL9_1DMBA_8Cys | DMBA-Met-Ala-Pro-Arg-Thr-Leu-Cys-Leu-COOH | 292 |
| VL9_1HMPA_8Cys | HMPA-Met-Ala-Pro-Arg-Thr-Leu-Cys-Leu-COOH | 293 |
| VL9_1Val(NMe)_8Cys | NMeH-Val-Met-Ala-Pro-Arg-Thr-Leu-Cys-Leu-COOH | 294 |
| VL9_8Dab | NH2-Val-Met-Ala-Pro-Arg-Thr-Leu-Dab-Leu-COOH | 295 |
| VL9_8Dap | NH2-Val-Met-Ala-Pro-Arg-Thr-Leu-Dap-Leu-COOH | 296 |
| VL9_1HMBA_8Cys | HMBA-Met-Ala-Pro-Arg-Thr-Leu-Cys-Leu-COOH | 297 |
| VL9_5Cys(FAM) | NH2-Val-Met-Ala-Pro-Cys(FAM)-Thr-Leu-Phe-Leu-COOH | 298 |
| VL9_2Nle_8Cys | NH2-Val-Nle-Ala-Pro-Arg-Thr-Leu-Cys-Leu-COOH | 299 |
| VL9_2Nle_8hCys | NH2-Val-Nle-Ala-Pro-Arg-Thr-Leu-hCys-Leu-COOH | 300 |
| VL9_2Nle_5Pgl_8Cys | NH2-Val-Nle-Ala-Pro-Pgl-Thr-Leu-Cys-Leu-COOH | 301 |
| VL9_2Nle_5Bhp_8Cys | NH2-Val-Nle-Ala-Pro-Bhp-Thr-Leu-Cys-Leu-COOH | 302 |
| VL9_scrambled_8Cys | NH2-Pro-Arg-Leu-Met-Ala-Leu-Val-Cys-Thr-COOH | 303 |
| VL9_scrambled_8Cys(mSF) | NH2-Pro-Arg-Leu-Met-Ala-Leu-Val-Cys(mSF)-Thr-COOH | 304 |
| VL9_scrambled_5Cys | NH2-Pro-Arg-Leu-Met-Cys-Leu-Val-Phe-Thr-COOH | 305 |
| VL9_scrambled_5Cys(FAM) | NH2-Pro-Arg-Leu-Met-Cys(FAM)-Leu-Val-Phe-Thr-COOH | 306 |
| VL9_5Ala_8Cys | NH2-Val-Met-Ala-Pro-Ala-Thr-Leu-Cys-Leu-COOH | 307 |
| VL9_5Lys_8Cys | NH2-Val-Met-Ala-Pro-Lys-Thr-Leu-Cys-Leu-COOH | 308 |
| VL9_5hArg_8Cys | NH2-Val-Met-Ala-Pro-hArg-Thr-Leu-Cys-Leu-COOH | 309 |
| VL9_5Cav_8Cys | NH2-Val-Met-Ala-Pro-Cav-Thr-Leu-Cys-Leu-COOH | 310 |
| VL9_5DArg_8Cys | NH2-Val-Met-Ala-Pro-DArg-Thr-Leu-Cys-Leu-COOH | 311 |
| VL9R_9Ala | NH2-Val-Met-Ala-Pro-Arg-Thr-Leu-Cys-Ala-COOH | 312 |
| VL9R_7Ala | NH2-Val-Met-Ala-Pro-Arg-Thr-Ala-Cys-Leu-COOH | 313 |
| VL9R_6Ala | NH2-Val-Met-Ala-Pro-Arg-Ala-Leu-Cys-Leu-COOH | 314 |
| VL9R_5Ala | NH2-Val-Met-Ala-Pro-Ala-Thr-Leu-Cys-Leu-COOH | 315 |
| VL9R_4Ala | NH2-Val-Met-Ala-Ala-Arg-Thr-Leu-Cys-Leu-COOH | 316 |
| VL9R_3Gly | NH2-Val-Met-Gly-Pro-Arg-Thr-Leu-Cys-Leu-COOH | 317 |
| VL9R_2Ala | NH2-Val-Ala-Ala-Pro-Arg-Thr-Leu-Cys-Leu-COOH | 318 |
| VL9R_1Ala | NH2-Ala-Met-Ala-Pro-Arg-Thr-Leu-Cys-Leu-COOH | 319 |
| 1(Bio) | NH2-Ala-Leu-Cys-Ser-Arg-Val-Pro-Cys-Ser-Gly-Gly-Lys(Bio)-CONH2 | 320 |
| 2(Bio) | NH2-Ala-Leu-Cys-Glu-Pro-Ser-Leu-Cys-Leu-Gly-Gly-Lys(Bio)-CONH2 | 321 |
| 3(Bio) | NH2-Ala-Leu-Cys-Gly-Arg-Ser-Cys-Leu-Val-Gly-Gly-Lys(Bio)-CONH2 | 322 |
| 4(Bio) | NH2-Ala-Leu-Cys-Gly-Leu-Ser-Cys-Leu-Val-Gly-Gly-Lys(Bio)-CONH2 | 323 |
| 5(Bio) | NH2-Ala-Leu-Cys-Gly-Ala-Ser-Cys-Leu-Phe-Gly-Gly-Lys(Bio)-CONH2 | 324 |
| 6(Bio) | NH2-Ala-Leu-Cys-Gly-Ser-Arg-Cys-Leu-Phe-Gly-Gly-Lys(Bio)-CONH2 | 325 |
| 7(Bio) | NH2-Ala-Leu-Cys-Gly-Ser-Ala-Cys-Leu-Tyr-Gly-Gly-Lys(Bio)-CONH2 | 326 |
| 8(Bio) | NH2-Ala-Leu-Cys-Gly-Arg-His-Cys-Leu-Phe-Gly-Gly-Lys(Bio)-CONH2 | 327 |
| 9(Bio) | NH2-Ala-Leu-Cys-Gly-Arg-Thr-Cys-Leu-Thr-Gly-Gly-Lys(Bio)-CONH2 | 328 |
| 10(Bio) | NH2-Ala-Leu-Cys-Gly-Arg-Ala-Cys-Leu-Trp-Gly-Gly-Lys(Bio)-CONH2 | 329 |
| 11(Bio) | NH2-Ala-Met-Cys-Gly-Arg-Leu-Cys-Leu-Phe-Gly-Gly-Lys(Bio)-CONH2 | 330 |
| 12(Bio) | NH2-Ala-Met-Cys-Gly-Leu-Pro-Cys-Leu-Val-Gly-Gly-Lys(Bio)-CONH2 | 331 |
| B11.1_8Cys(mSF) | NH2-DhAr-Nle-hAr-Gln-Mff-Ala(NMe)-Cha-Cys(mSF)-Leu-COOH | 332 |
| B11.8_8Cys(mSF) | NH2-DhAr-Nle-hAr-Gln-Mff-Ala-Cha-Cys(mSF)-Leu-COOH | 333 |
| P2(FITC) | NH2-Ala-Leu-Cys-Trp-Arg-Val-Pro-Cys-Thr-Gly-Gly-Lys(FITC)-CONH2 | 334 |
| P4(FITC) | NH2-Ala-Met-Cys-Pro-Pro-Trp-Met-Cys-Phe-Gly-Gly-Lys(FITC)-CONH2 | 335 |
| P6(FITC) | NH2-Ala-Met-Cys-Gly-Ala-Arg-Cys-Leu-Phe-Gly-Gly-Lys(FITC)-CONH2 | 336 |
| P1.1 | NH2-Ala-Nle-Cys-Gly-Pro-Arg-Cys-Leu-Trp-Gly-Gly-Lys(FITC)-CONH2 | 337 |
| P1.5 | NH2-Met-Cys-Gly-Pro-Arg-Cys-Leu-Trp-Gly-Gly-Lys(FITC)-CONH2 | 338 |
| P18(FITC) | NH2-Ala-Leu-Cys-Trp-Arg-Val-Ser-Cys-Thr-Gly-Gly-Lys(FITC)-CONH2 | 339 |
| P1_1Gly(FITC) | NH2-Gly-Met-Cys-Gly-Pro-Arg-Cys-Leu-Trp-Gly-Gly-Lys(FITC)-CONH2 | 340 |
| P1_2Ala(FITC) | NH2-Ala-Ala-Cys-Gly-Pro-Arg-Cys-Leu-Trp-Gly-Gly-Lys(FITC)-CONH2 | 341 |
| P1_4Ala(FITC) | NH2-Ala-Met-Cys-Ala-Pro-Arg-Cys-Leu-Trp-Gly-Gly-Lys(FITC)-CONH2 | 342 |
| P1_5Ala(FITC) | NH2-Ala-Met-Cys-Gly-Ala-Arg-Cys-Leu-Trp-Gly-Gly-Lys(FITC)-CONH2 | 343 |
| P1_6Ala(FITC) | NH2-Ala-Met-Cys-Gly-Pro-Ala-Cys-Leu-Trp-Gly-Gly-Lys(FITC)-CONH2 | 344 |
| P1_8Ala(FITC) | NH2-Ala-Met-Cys-Gly-Pro-Arg-Cys-Ala-Trp-Gly-Gly-Lys(FITC)-CONH2 | 345 |
| P1_9Ala(FITC) | NH2-Ala-Met-Cys-Gly-Pro-Arg-Cys-Leu-Ala-Gly-Gly-Lys(FITC)-CONH2 | 346 |
| T1_Cbio | NH2-Gln-Gln-Phe-DPro-Gly-Asn-Pro-Asn-Gly-Asn-Ala-Leu-Asp-Glu-Ser-Abu-Lys(biotin)- | 347 |
| | CONH2 | |
| T2_Cbio | NH2-Gln-Gln-Phe-DPro-Pro-Asn-Pro-Asn-Gly-Asn-Ala-Leu-Asp-Glu-Ser-Abu-Lys(biotin)- | 348 |
| | CONH2 | |
| T3_Cbio | NH2-Gln-Gln-Phe-Ala-Aib-Asn-Pro-Asn-Gly-Asn-Ala-Leu-Asp-Glu-Ser-Abu-Lys(biotin)- | 349 |
| | CONH2 | |
| B9_1Cys(mSF) | NH2-Cys(mSF)-Nle-hAr-Gln-Mff-Ala-Cha-hAr-Leu-COOH | 350 |
| B9_1Cys(pSF) | NH2-Cys(pSF)-Nle-hAr-Gln-Mff-Ala-Cha-hAr-Leu-COOH | 351 |
| B9_scrambled | NH2-Cha-hAr-Ala-Leu-hAr-Gln-Nle-Mff-hAr-COOH | 352 |
| VL9_8Cys(mOSF) | NH2-Val-Met-Ala-Pro-Arg-Thr-Leu-Cys(mOSF)-Leu-COOH | 353 |
| VL9-GGK(FITC) | NH2-Val-Met-Ala-Pro-Ala-Thr-Leu-Phe-Leu-Gly-Gly-Lys(FITC)-CONH2 | 354 |

### INCORPORATION BY REFERENCE

The entire disclosure of each of the patent and scientific documents referred to herein is incorporated by reference for all purposes.

### EQUIVALENTS

An invention may be embodied in other specific forms without departing from the spirit or essential characteristics thereof. The foregoing embodiments are therefore to be considered in all respects illustrative rather than limiting on any invention disclosed herein. Scope of an invention is thus indicated by the appended claims rather than by the foregoing description, and all changes that come within the meaning and range of equivalency of the claims are intended to be embraced therein.

SEQUENCE LISTING SUMMARY

| SEQ | |
|---|---|
| ID | |
| NO: | Sequence |
| 1 | VMAPRT(L/V)(V/L/I/F)L |
| 2 | VMAPRTLVL |
| 3 | X-Met-X-X-Z-Ala-X-U-Leu |
| 4 | X-Met-X-X-Z-Ala-X-U-Leu-Lys |
| 5 | NH2-4Af-Met-Dff-Ser-Ala-Ala-Cha-Arg-Leu-OH |
| 6 | NH2-hAr-Met-Msn-Dff-Cha-Ala-Arg-Msn-Leu-OH |
| 7 | NH2-Ala-Met-hAr-Dff-Tha-Ala-Cha-Arg-Leu-OH |
| 8 | NH2-4Af-Met-Ala-Ser-Ala-Ala-Cha-Arg-Leu-OH |
| 9 | NH2-hAR-Met-hAR-Gln-Mff-Ala-Cha-hAR-Leu-OH |
| 10 | NH2-4Af-Met-Dff-Ser-Ala-Ala-Cha-Cys-Leu-OH |
| 11 | NH2-hAr-Met-Msn-Dff-Cha-Ala-Arg-Cys-Leu-OH |
| 12 | NH2-Ala-Met-hAr-Dff-Tha-Ala-Cha-Cys-Leu-OH |
| 13 | NH2-4Af-Met-Ala-Ser-Ala-Ala-Cha-Cys-Leu-OH |
| 14 | NH2-hAR-Met-hAR-Gln-Mff-Ala-Cha-Cys-Leu-OH |
| 15 | NH2-4Af-Met-Cys-Ser-Ala-Ala-Cha-Arg-Leu-OH |
| 16 | NH2-hAr-Met-Cys-Dff-Cha-Ala-Arg-Msn-Leu-OH |
| 17 | NH2-Ala-Met-Cys-Dff-Tha-Ala-Cha-Arg-Leu-OH |
| 18 | NH2-4Af-Met-Cys-Ser-Ala-Ala-Cha-Arg-Leu-OH |
| 19 | NH2-hAR-Met-Cys-Gln-Mff-Ala-Cha-hAR-Leu-OH |
| 20 | hAr-X-hAr-Gln-Mff-A-Cha-hAr-Z |
| 21 | NH2-hAr-Nle-hAr-Gln-Mff-Ala-Cha-hAr-Leu-OH |
| 22 | NH2-hAr-Mox-hAr-Gln-Mff-Ala-Cha-hAr-Leu-OH |
| 23 | NH₂-hAr-Met-hAr-Gln-Mff-Ala-Cha-hAr-Nle-OH |
| 24 | NH2-hAr-Nle-hAr-Gln-Mff-Ala-Cha-hAr-Aca-OH |
| 25 | NH2-hAr-Nle-hAr-Gln-Mff-Ala-Cha-hAr-Cha-OH |
| 26 | NH2-hAr-Nle-Cys-Gln-Mff-Ala-Cha-hAr-Leu-OH |
| 27 | NH2-hAr-Nle-hAr-Gln-Mff-Ala-Cha-Cys-Leu-OH |
| 28 | NH2-hAr-Mox-Cys-Gln-Mff-Ala-Cha-hAr-Leu-OH |
| 29 | NH2-hAr-Mox-hAr-Gln-Mff-Ala-Cha-Cys-Leu-OH |
| 30 | NH₂-hAr-Met-Cys-Gln-Mff-Ala-Cha-hAr-Nle-OH |
| 31 | NH₂-hAr-Met-hAr-Gln-Mff-Ala-Cha-Cys-Nle-OH |
| 32 | NH2-hAr-Cys-hAr-Gln-Mff-Ala-Cha-hAr-Aca-OH |
| 33 | NH2-hAr-Nie-hAr-Gln-Mff-Ala-Cha-Cys-Aca-OH |
| 34 | NH2-hAr-Nle-Cys-Gln-Mff-Ala-Cha-hAr-Cha-OH |
| 35 | NH2-hAr-Nle-hAr-Gln-Mff-Ala-Cha-Cys-Cha-OH |
| 36 | VMAPRTLFL |
| 37 | NH₂-Cys(mSF)-Met-Ala-Pro-Arg-Thr-Leu-Phe-Leu-OH |
| 38 | NH₂-Val-Cys(mSF)-Ala-Pro-Arg-Thr-Leu-Phe-Leu-OH |
| 39 | NH₂-Val-Met-Cys(mSF)-Pro-Arg-Thr-Leu-Phe-Leu-OH |
| 40 | NH₂-Val-Met-Ala-Cys(mSF)-Arg-Thr-Leu-Phe-Leu-OH |
| 41 | NH₂-Val-Met-Ala-Pro-Cys(mSF)-Thr-Leu-Phe-Leu-OH |
| 42 | NH₂-Val-Met-Ala-Pro-Arg-Cys(mSF)-Leu-Phe-Leu-OH |
| 43 | NH₂-Val-Met-Ala-Pro-Arg-Thr-Cys(mSF)-Phe-Leu-OH |
| 44 | NH₂-Val-Met-Ala-Pro-Arg-Thr-Leu-Cys(mSF)-Leu-OH |
| 45 | NH₂-Val-Met-Ala-Pro-Arg-Thr-Leu-Phe-Cys(mSF)-OH |
| 46 | NH₂-hAr-Nle-hAr-Gln-Mff-Ala-Cha-Cys(mSF)-Leu-OH |
| 47 | VMAP(Anpp)TLFL |
| 48 | MGSHSLKYFHTSVSRPGRGEPRFISVGYVDDTQFVREDNDAASPRMVPRAPWMEQEGSEYWDRETRSARD |
| | TAQIFRVNLRTLRGYYNQSEAGSHTLQWMHGCELGPDGRFLRGYEQFAYDGKDYLTLNEDLRSWTAVDTA |
| | AQISEQKSNDASEAEHQRAYLEDTCVEWLHKYLEKGKETLLHLEPPKTHVTHHPISDHEATLRCWALGFY |
| | PAEITLTWQQDGEGHTQDTELVETRPAGDGTFQKWAAVVVPSGEEQRYTCHVQHEGLPEPVTLRWKPASQ |
| | PTIPIGSGLNDIFEAQKIEWHE |
| | [HLAE(hu)(22-305)]-Avitag] |
| 49 | MIQRTPKIQVYSRHPAENGKSNFLNCYVSGFHPSDIEVDLLKNGERIEKVEHSDLSFSKDWSFYLLYYTE |
| | FTPTEKDEYACRVNHVTLSQPKIVKWDRDM |
| | [B2M(hu)(21-119)] |
| 50 | GHHHHHHHHGGKNSFTKLSIEPAFTPGPNIELQKDSDCCSCQEKWVGYRCNCYFISSEQKTWNESRHLCA |
| | SQKSSLLQLQNTDELDFMSSSQQFYWIGLSYSEEHTAWLWENGSALSQYLFPSFETFNTKNCIAYNPNGN |
| | ALDESCEDKNRYICKQQLIGSGGGGSGGGGSGGGGSGGGGSGGGGSGGGGSGGPSTLIQRHNNSSLNTRT |
| | QKARHCGHCPEEWITYSNSCYYIGKERRTWEESLLACTSKNSSLLSIDNEEEMKFLSIISPSSWIGVFRN |
| | SSHHPWVTMNGLAFKHEIKDSDNAELNCAVLQVNRLKSAQCGSSIIYHCKHKL |
| | 8xHis-hSCD(CD94 K32-I179/NKG2A P94-L333) |
| 51 | GGLNDIFEAQKIEAHEAPEAAGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQVYTKPPS |
| | RDELTKNQVSLSCLVKGFYPSDIAVEWESNGQPENNYKTTVPVLDSDGSFRLASYLTVDKSRWQQGNVFS |
| | CSVMHEALHNHYTQKSLSLSPGGGGKNSFTKLSIEPAFTPGPNIELQKDSDCCSCQEKWVGYRCNCYFIS |
| | SEQKTWNESRHLCASQKSSLLQLQNTDELDFMSSSQQFYWIGLSYSEEHTAWLWENGSALSQYLFPSFET |
| | FNTKNCIAYNPNGNALDESCEDKNRYICKQQLIGSGGGGSGGGGSGGGGSGGGGSGGGGSGGGGSGGPST |
| | LIQRHNNSSLNTRTQKARHCGHCPEEWITYSNSCYYIGKERRTWEESLLACTSKNSSLLSIDNEEEMKFL |
| | SIISPSSWIGVERNSSHHPWVTMNGLAFKHEIKDSDNAELNCAVLQVNRLKSAQCGSSIIYHCKHKL |
| | AviTag-oaFC-hSCD(CD94 K32-I179/NKG2A P94-L333) |
| 52 | Lys(TAMRA)-Nle-hAr-Gln-Mff-Ala-Cha-hAr-Leu |
| 53 | hAr-Nle-hAr-Gln-Lys(TAMRA)-Ala-Cha-hAr-Leu |
| 54 | hAr-Nle-hAr-Gln-Mff-Ala-Cha-Lys(TAMRA)-Leu |
| 55 | hAr-Nle-hAr-Gln-Mff-Ala-Cha-hAr-Leu-Leu-Pro-Glu-Thr-Gly-Gly |
| 56 | hAr-Nle-hAr-Gln-Lys(DOTA)-Ala-Cha-hAr-Leu |
| 57 | hAr-Nle-hAr-Gln-Lys(ibu)-Ala-Cha-hAr-Leu |
| 58 | hAr-Nle-hAr-Gln-Lys(FITC)-Ala-Cha-hAr-Leu |
| 59 | hAr-Nle-hAr-Gln-Mff-Ala-Cha-Lys(ibu)-Leu |
| 60 | hAr-Nle-hAr-Gln-Lys(CUM)-Ala-Cha-hAr-Leu |
| 61 | Ala-Cha-Leu-Ala-Met-Arg-4Af-Ala-Ser |
| 62 | Gln-Gln-Phe-DPro-Gly-Asn-Pro-Asn-Gly-Asn-Ala-Leu-Asp-Glu-Ser-CONH2 |
| 63 | Gln-Gln-Phe-DPro-Pro-Asn-Pro-Asn-Gly-Asn-Ala-Leu-Asp-Glu-Ser-CONH2 |
| 64 | Gln-Gln-Phe-Ala-Aib-Asn-Pro-Asn-Gly-Asn-Ala-Leu-Asp-Glu-Ser-CONH2 |
| 65 | PEG4-Asn-Ala-Leu-Asp-Glu-Tyr-Cys-Glu-Asp-Lys-Asn-Arg-CONH2 |
| 66 | Asn-Ala-Leu-Asp-Glu-Tyr-Cys-Glu-Asp-Lys-Asn-Arg-PEG2-Lys(biotin)-CONH2 |
| 67 | Asn-Ala-Leu-Asp-Glu-Tyr-Lys(PEG4-biotin)-Glu-Asp-Lys-Asn-Arg-CONH2 |
| 68 | Val-Nle-Ala-Pro-Dap(Ser-Ile-Cys(ox.)-His-Gly-Ile-Glu-Tyr-Pro- |
| | Cys(ox.))-Thr-Leu-Phe-Leu |
| 69 | Val-Nle-Ala-Pro-Dap(Ser-Pro-Cys(ox.)-Glu-Arg-Trp-Ala-Ile-Ser-Phe- |
| | Cys(ox.))-Thr-Leu-Phe-Leu |
| 70 | Val-Nle-Ala-Pro-Dap(Ser-His-Cys(ox.)-Ser-Asp-Thr-Gln-Glu-Ala-Tyr- |
| | Cys(ox.))-Thr-Leu-Phe-Leu |
| 71 | Val-Nle-Ala-Pro-Dap(Ser-Trp-Cys(ox.)-Thr-Lys-Thr-Asn-Asp-Gln-Arg- |
| | Cys(ox.))-Thr-Leu-Phe-Leu |
| 72 | Val-Nle-Ala-Pro-Dap(Ser-Phe-Cys(ox.)-Asn-Ile-Ser-Gly-Gly-Ile-His- |
| | Cys(ox.))-Thr-Leu-Phe-Leu |
| 73 | Ala-Nle-hAr-Gln-Mff-Ala-Cha-hAr-Leu |
| 74 | hAr-Ala-hAr-Gln-Mff-Ala-Cha-hAr-Leu |
| 75 | hAr-Nle-Ala-Gln-Mff-Ala-Cha-hAr-Leu |
| 76 | hAr-Nle-hAr-Ala-Mff-Ala-Cha-hAr-Leu |
| 77 | hAr-Nle-hAr-Gln-Ala-Ala-Cha-hAr-Leu |
| 78 | hAr-Nle-hAr-Gln-Mff-Gly-Cha-hAr-Leu |
| 79 | hAr-Nle-hAr-Gln-Mff-Ala-Ala-hAr-Leu |
| 80 | hAr-Nle-hAr-Gln-Mff-Ala-Cha-Ala-Leu |
| 81 | hAr-Nle-hAr-Gln-Mff-Ala-Cha-hAr-Ala |
| 82 | DhAr-Nle-DhAr-Gln-Mff-Ala(NMe)-Cha-DhAr-Leu |
| 83 | DhAr-Nle-hAr-Gln-Mff-Ala(NMe)-Cha-DhAr-Leu |
| 84 | DhAr-Nle-hAr-Gln-Mff-Ala-Cha-hAr-Leu |
| 85 | hAr-Nle-DhAr-Gln-Mff-Ala-Cha-hAr-Leu |
| 86 | hAr-Nle-hAr-Gln-Mff-Ala-Cha-DhAr-Leu |
| 87 | hAr-Nle-hAr-Gln-Mff-Bal-Cha-hAr-Leu |
| 88 | hAr-Nle-hAr-Gln-Mff-Ala(NMe)-Cha-hAr-Leu |
| 89 | hAr-Nle-hAr-Gln-Mff-Ala(NMe)-Cha-DhAr-Leu |
| 90 | DhAr-Nle-hAr-Gln-Mff-Ala-Cha-DhAr-Leu |
| 91 | DhAr-Nle-hAr-Gln-Mff-Ala(NMe)-Cha-hAr-Leu |
| 92 | Ala-Met-Cys-Gly-Pro-Arg-Cys-Leu-Trp-CONH2 |
| 93 | Ala-Leu-Cys-Trp-Arg-Val-Pro-Cys-Thr-CONH2 |
| 94 | Ala-Met-Cys-Pro-Pro-Ser-Leu-Cys-Phe-CONH2 |
| 95 | Ala-Met-Cys-Pro-Pro-Trp-Met-Cys-Phe-CONH2 |
| 96 | Ala-Leu-Cys-Gly-Arg-Ser-Cys-Leu-Val-CONH2 |
| 97 | Ala-Met-Cys-Gly-Ala-Arg-Cys-Leu-Phe-CONH2 |
| 98 | Ala-Met-Cys-Gly-Arg-Leu-Cys-Leu-Phe-CONH2 |
| 99 | Ala-Leu-Cys-Thr-Arg-Val-Pro-Cys-Thr-CONH2 |
| 100 | Ala-Leu-Cys-Gly-Lys-Ala-Cys-Leu-Tyr-CONH2 |
| 101 | Ala-Leu-Cys-Gly-Ala-Arg-Cys-Leu-Phe-CONH2 |
| 102 | Ala-Met-Cys-Trp-Ser-Val-Pro-Cys-Ser-CONH2 |
| 103 | Ala-Leu-Cys-Pro-Arg-Val-Pro-Cys-Ser-CONH2 |
| 104 | Ala-Met-Cys-Arg-Gln-Val-Pro-Cys-His-CONH2 |
| 105 | Ala-Leu-Cys-Ser-Arg-Val-Pro-Cys-Ser-CONH2 |
| 106 | Ala-Leu-Cys-Gly-Arg-Ala-Cys-Leu-Trp-CONH2 |
| 107 | Gly-hAr-Nle-hAr-Gln-Mff-Ala-Cha-hAr-Leu |
| 108 | Gly-Gly-Gly-hAr-Nle-hAr-Gln-Mff-Ala-Cha-hAr-Leu |
| 109 | Gly-Gly-Gly-Gly-Gly-hAr-Nle-hAr-Gln-Mff-Ala-Cha-hAr-Leu |
| 110 | COCH3-hAr-Nle-hAr-Gln-Mff-Ala-Cha-Cys(pSF)-Leu |
| 111 | COCH3-hAr-Nle-hAr-Gln-Mff-Ala-Cha-Cys(pPC)-Leu |
| 112 | COCH3-hAr-Nle-hAr-Gln-Mff-Ala-Cha-Lys(Tz-SQ)-Leu |
| 113 | COCH3-hAr-Nle-hAr-Gln-Mff-Ala-Cha-Cys(mSF)-Leu |
| 114 | Cys(mSF)-Met-Ala-Pro-Arg-Thr-Leu-Phe-Leu |
| 115 | Val-Cys(mSF)-Ala-Pro-Arg-Thr-Leu-Phe-Leu |
| 116 | Val-Met-Cys(mSF)-Pro-Arg-Thr-Leu-Phe-Leu |
| 117 | Val-Met-Ala-Cys(mSF)-Arg-Thr-Leu-Phe-Leu |
| 118 | Val-Met-Ala-Pro-Cys(mSF)-Thr-Leu-Phe-Leu |
| 119 | Val-Met-Ala-Pro-Arg-Cys(mSF)-Leu-Phe-Leu |
| 120 | Val-Met-Ala-Pro-Arg-Thr-Cys(mSF)-Phe-Leu |
| 121 | Val-Met-Ala-Pro-Arg-Thr-Leu-Phe-Cys(mSF) |
| 122 | Val-Met-Cys(mSF)-Pro-Arg-Thr-Leu-Cys(mSF)-Leu |
| 123 | hAr-Nle-Lys(Tz-SQ)-Gln-Mff-Ala-Cha-hAr-Leu |
| 124 | hAr-Nle-Lys(Tz-PTAD)-Gln-Mff-Ala-Cha-hAr-Leu |
| 125 | hAr-Nle-hAr-Gln-Mff-Ala-Cha-Lys(Tz-SQ)-Leu |
| 126 | hAr-Nle-hAr-Gln-Mff-Ala-Cha-Lys(Tz-PTAD)-Leu |
| 127 | Cha-hAr-Ala-Leu-hAr-Gln-Nle-Cys(mSF)-hAr |
| 128 | Val-Met-Ala-Pro-Arg-Thr-Leu-Dha-Leu |
| 129 | Val-Met-Ala-Pro-Arg-Thr-Leu-Cys(mSF)-Leu |
| 130 | COCH3-Cys(mSF)-Nle-hAr-Gln-Mff-Ala-Cha-hAr-Leu |
| 131 | COCH3-hAr-Cys(mSF)-hAr-Gln-Mff-Ala-Cha-hAr-Leu |
| 132 | COCH3-Dap(Tz-PTAD)-Nle-hAr-Gln-Mff-Ala-Cha-hAr-Leu |
| 133 | COCH3-hAr-Dap(Tz-PTAD)-hAr-Gln-Mff-Ala-Cha-hAr-Leu |
| 134 | hAr-Nle-hAr-Gln-Mff-Ala-Cha-Cys(pSF)-Leu |
| 135 | hAr-Nle-hAr-Gln-Mff-Ala-Cha-Cys(mSF)-Leu |
| 136 | hAr-Nle-hAr-Gln-Mff-Ala-Cha-Cys(pPC)-Leu |
| 137 | hAr-Nle-hAr-Gln-Lys(PEG2-FITC)-Ala-Cha-Cys-Leu |
| 138 | Cha-Cys(mSF)-Ala-Leu-hAr-Gln-Nle-Mff-hAr |
| 139 | hAr-Nle-Cys(mSF)-Gln-Mff-Ala-Cha-hAr-Leu |
| 140 | Val-Met-Ala-Pro-Lys(PEG2-DOTA)-Thr-Leu-Cys(mSF)-Leu |
| 141 | Val-Met-Ala-Pro-Lys(PEG2-FITC)-Thr-Leu-Cys(mSF)-Leu |
| 142 | Val-Met-Cys(mSF)-Pro-Lys(DOTA)-Thr-Leu-Phe-Leu |
| 143 | Val-Met-Cys(mSF)-Pro-Lys(PEG4-biotin)-Thr-Leu-Phe-Leu |
| 144 | Val-Met-Cys(mSF)-Pro-Lys(PEG2-ibu)-Thr-Leu-Phe-Leu |
| 145 | Val-Met-Ala-Pro-Lys(PEG2-ibu)-Thr-Leu-Cys(mSF)-Leu |
| 146 | Val-Met-Ala-Pro-Lys(PEG4-biotin)-Thr-Leu-Cys(mSF)-Leu |
| 147 | Val-Met-Ala-Pro-Lys(Tz-CD94[111-114])-Thr-Leu-Cys(mSF)-Leu |
| 148 | Val-Met-Lys(Tz-NKG2A[213-217])-Pro-Arg-Thr-Leu-Cys(mSF)-Leu |
| 149 | Val-Met-Ala-Pro-Arg-Thr-Leu-Cys(pPC)-Leu |
| 150 | Val-Met-Ala-Pro-Arg-Thr-Leu-Cys(pSF)-Leu |
| 151 | Val-Met-Ala-Pro-Arg-Thr-Leu-Cys(NHS)-Leu |
| 152 | Val-Met-Ala-Pro-Arg-Thr-Leu-Cys(mPC)-Leu |
| 153 | Val-Met-Ala-Pro-Arg-Thr-Leu-Cys(mSN)-Leu |
| 154 | Val-Met-Ala-Pro-Arg-Thr-Leu-Cys(pSN)-Leu |
| 155 | DMBA-Met-Ala-Pro-Arg-Thr-Leu-Cys(mSF)-Leu |
| 156 | HMPA-Met-Ala-Pro-Arg-Thr-Leu-Cys(mSF)-Leu |
| 157 | NMeH-Val-Met-Ala-Pro-Arg-Thr-Leu-Cys(mSF)-Leu |
| 158 | Val-Met-Ala-Pro-Arg-Thr-Leu-Dab(3SF)-Leu |
| 159 | Val-Met-Ala-Pro-Arg-Thr-Leu-Dab(4SF)-Leu |
| 160 | Val-Met-Ala-Pro-Arg-Thr-Leu-Dab(3SF4OMe)-Leu |
| 161 | Val-Met-Ala-Pro-Arg-Thr-Leu-Dab(5SF2OMe)-Leu |
| 162 | Val-Met-Ala-Pro-Arg-Thr-Leu-Dap(3SF)-Leu |
| 163 | Val-Met-Ala-Pro-Arg-Thr-Leu-Dap(4SF)-Leu |
| 164 | Val-Met-Ala-Pro-Arg-Thr-Leu-Dap(3SF4OMe)-Leu |
| 165 | Val-Met-Ala-Pro-Arg-Thr-Leu-Dap(5SF2OMe)-Leu |
| 166 | HMBA-Met-Ala-Pro-Arg-Thr-Leu-Cys(mSF)-Leu |
| 167 | Val-Met-Ala-Pro-Arg-Thr-Leu-Cys(mFSF)-Leu |
| 168 | Val-Met-Ala-Pro-Arg-Thr-Leu-Cys(mMeSF)-Leu |
| 169 | Val-Met-Ala-Pro-Arg-Thr-Leu-Cys(mPySF)-Leu |
| 170 | Val-Met-Ala-Pro-Arg-Thr-Leu-Cys(SAL)-Leu |
| 171 | Val-Nle-Ala-Pro-Arg-Thr-Leu-Cys(mSF)-Leu |
| 172 | Val-Nle-Ala-Pro-Arg-Thr-Leu-hCys(mSF)-Leu |
| 173 | Val-Nle-Ala-Pro-Pgl-Thr-Leu-Cys(mSF)-Leu |
| 174 | Val-Nle-Ala-Pro-Bhp-Thr-Leu-Cys(mSF)-Leu |
| 175 | Val-Met-Ala-Pro-Ala-Thr-Leu-Cys(mSF)-Leu |
| 176 | Val-Met-Ala-Pro-Lys-Thr-Leu-Cys(mSF)-Leu |
| 177 | Val-Met-Ala-Pro-DArg-Thr-Leu-Cys(mACA)-Leu |
| 178 | Val-Met-Ala-Pro-DArg-Thr-Leu-Cys(mSAC)-Leu |
| 179 | Val-Met-Ala-Pro-Arg-Thr-Leu-Cys(mAld)-Leu |
| 180 | Val-Met-Ala-Pro-Arg-Thr-Leu-Cys(oAld)-Leu |
| 181 | Val-Met-Ala-Pro-hArg-Thr-Leu-Cys(mSF)-Ala |
| 182 | Val-Met-Ala-Pro-Cav-Thr-Leu-Cys(mSF)-Ala |
| 183 | Val-Met-Ala-Pro-DArg-Thr-Leu-Cys(mSF)-Ala |
| 184 | Val-Met-Ala-Pro-Arg-Thr-Leu-Cys(mSF)-Ala |
| 185 | Val-Met-Ala-Pro-Arg-Thr-Ala-Cys(mSF)-Leu |
| 186 | Val-Met-Ala-Pro-Arg-Ala-Leu-Cys(mSF)-Leu |
| 187 | Val-Met-Ala-Pro-Ala-Thr-Leu-Cys(mSF)-Leu |
| 188 | Val-Met-Ala-Ala-Arg-Thr-Leu-Cys(mSF)-Leu |
| 189 | Val-Ala-Ala-Pro-Arg-Thr-Leu-Cys(mSF)-Leu |
| 190 | Ala-Met-Ala-Pro-Arg-Thr-Leu-Cys(mSF)-Leu |
| 191 | Val-Met-Gly-Pro-Arg-Thr-Leu-Cys(mSF)-Leu |
| 192 | dhAr-Nle-lhAr-Gln-Mff-Ala(N-Methyl)-Cha-Cys(mSF)-Leu |
| 193 | dhAr-Nle-lhAr-Gln-Mff-Ala-Cha-Cys(mSF)-Leu |
| 194 | Val-Met-Ala-Pro-Lys(FITC)-Thr-Leu-Phe-Leu |
| 195 | Ala-Met-Cys-Gly-Pro-Arg-Cys-Leu-Trp-Gly-Gly-Lys(FITC) |
| 196 | Ala-Met-Cys-Pro-Pro-Ser-Leu-Cys-Phe-Gly-Gly-Lys(FITC) |
| 197 | Ala-Leu-Cys-Gly-Arg-Ser-Cys-Leu-Val-Gly-Gly-Lys(FITC) |
| 198 | Ala-Met-Cys-Gly-Arg-Leu-Cys-Leu-Phe-Gly-Gly-Lys(FITC) |
| 199 | Ala-Leu-Cys-Thr-Arg-Val-Pro-Cys-Thr-Gly-Gly-Lys(FITC) |
| 200 | Ala-Leu-Cys-Gly-Lys-Ala-Cys-Leu-Tyr-Gly-Gly-Lys(FITC) |
| 201 | Ala-Leu-Cys-Gly-Ala-Arg-Cys-Leu-Phe-Gly-Gly-Lys(FITC) |
| 202 | Ala-Met-Cys-Trp-Ser-Val-Pro-Cys-Ser-Gly-Gly-Lys(FITC) |
| 203 | Ala-Leu-Cys-Pro-Arg-Val-Pro-Cys-Ser-Gly-Gly-Lys(FITC) |
| 204 | Ala-Met-Cys-Arg-Gln-Val-Pro-Cys-His-Gly-Gly-Lys(FITC) |
| 205 | Ala-Leu-Cys-Ser-Arg-Val-Pro-Cys-Ser-Gly-Gly-Lys(FITC) |
| 206 | Ala-Leu-Cys-Gly-Arg-Ala-Cys-Leu-Trp-Gly-Gly-Lys(FITC) |
| 207 | Ala-Met-Cys-Gly-Pro-Arg-Cys-Leu-Trp-Leu-Gly-Gly-Lys(FITC) |
| 208 | Ala-Met-Cys-Gly-Pro-Arg-Cys-Leu-Phe-Leu-Gly-Gly-Lys(FITC) |
| 209 | Ala-Met-Cys-Gly-Pro-Arg-Cys-Leu-Gly-Gly-Lys(FITC) |
| 210 | Ala-Met-Cys-Asn-Arg-Val-Pro-Cys-Thr-Gly-Gly-Lys(FITC) |
| 211 | Ala-Met-Cys-Pro-Thr-Ile-Pro-Cys-Ser-Gly-Gly-Lys(FITC) |
| 212 | Ala-Leu-Cys-Trp-Arg-Val-Arg-Cys-Thr-Gly-Gly-Lys(FITC) |
| 213 | Ala-Leu-Cys-Trp-Arg-Val-Pro-Cys-Thr-Gly-Gly-Lys(FITC) |
| 214 | Ala-Leu-Cys-Ala-Pro-Arg-Leu-Cys-Leu-Gly-Gly-Lys(FITC) |
| 215 | Ala-Leu-Cys-Asp-Pro-Ser-Lys-Cys-Phe-Gly-Gly-Lys(FITC) |
| 216 | Ala-Leu-Cys-Trp-His-Val-Pro-Cys-Gln-Gly-Gly-Lys(FITC) |
| 217 | Ala-Leu-Cys-Asp-Pro-Arg-Trp-Cys-Phe-Gly-Gly-Lys(FITC) |
| 218 | Ala-Met-Cys-Gly-Lys-Asn-Cys-Leu-Phe-Gly-Gly-Lys(FITC) |
| 219 | Ala-Leu-Cys-Gly-Arg-Pro-Cys-Leu-Tyr-Gly-Gly-Lys(FITC) |
| 220 | Ala-Met-Cys-Gly-Lys-Tyr-Cys-Leu-Phe-Gly-Gly-Lys(FITC) |
| 221 | Ala-Leu-Cys-Arg-Trp-Arg-Cys-Leu-Tyr-Gly-Gly-Lys(FITC) |
| 222 | Ala-Leu-Cys-Gly-Trp-Arg-Cys-Leu-Tyr-Gly-Gly-Lys(FITC) |
| 223 | Ala-Met-Cys-Val-Pro-Arg-Cys-Leu-Trp-Gly-Gly-Lys(FITC) |
| 224 | Ala-Leu-Cys-Gly-Ile-Ala-Cys-Phe-Pro-Gly-Gly-Lys(FITC) |
| 225 | Ala-Met-Cys-Gly-Ala-Leu-Cys-Leu-Phe-Gly-Gly-Lys(FITC) |
| 226 | Ala-Leu-Cys-Trp-Arg-Val-Pro-Cys-Thr |
| 227 | Ala-Leu-Cys-Ala-Pro-Arg-Leu-Cys-Leu |
| 228 | Ala-Leu-Cys-Asp-Pro-Arg-Trp-Cys-Phe |
| 229 | Ala-Leu-Cys-Gly-Trp-Arg-Cys-Leu-Tyr |
| 230 | Ala-Met-Cys-Gly-Ala-Leu-Cys-Leu-Phe |
| 231 | COCH3-hAr-Nle-hAr-Gln-Mff-Ala-Cha-Cys-Leu-COOH |
| 232 | NH2-Lys(biotin)-Nle-hAr-Gln-Mff-Ala-Cha-hAr-Leu-COOH |
| 233 | NH2-hAr-Nle-hAr-Gln-Lys(biotin)-Ala-Cha-hAr-Leu-COOH |
| 234 | NH2-hAr-Nle-hAr-Gln-Mff-Ala-Cha-Lys(biotin)-Leu-COOH |
| 235 | COCH3-Cys-Nle-hAr-Gln-Mff-Ala-Cha-hAr-Leu-COOH |
| 236 | COCH3-hAr-Cys-hAr-Gln-Mff-Ala-Cha-hAr-Leu-COOH |
| 237 | COCH3-Dap(N3)-Nle-hAr-Gln-Mff-Ala-Cha-hAr-Leu-COOH |
| 238 | COCH3-hAr-Dap(N3)-hAr-Gln-Mff-Ala-Cha-hAr-Leu-COOH |
| 239 | -hAr-Nle-hAr-Gln-Lys(DOTA)-Ala-Cha-Lys(ibu)-Leu-COOH |
| 240 | NH2-Cys-Nle-hAr-Gln-Mff-Ala-Cha-hAr-Leu-COOH |
| 241 | NH2-hAr-Nle-hAr-Gln-Mff-Ala-Cha-Cys-Leu-COOH |
| 242 | NH2-Cys-Met-Ala-Pro-Arg-Thr-Leu-Phe-Leu-COOH |
| 243 | NH2-Val-Cys-Ala-Pro-Arg-Thr-Leu-Phe-Leu-COOH |
| 244 | NH2-Val-Met-Cys-Pro-Arg-Thr-Leu-Phe-Leu-COOH |
| 245 | NH2-Val-Met-Ala-Cys-Arg-Thr-Leu-Phe-Leu-COOH |
| 246 | NH2-Val-Met-Ala-Pro-Cys-Thr-Leu-Phe-Leu-COOH |
| 247 | NH2-Val-Met-Ala-Pro-Arg-Cys-Leu-Phe-Leu-COOH |
| 248 | NH2-Val-Met-Ala-Pro-Arg-Thr-Cys-Phe-Leu-COOH |
| 249 | NH2-Val-Met-Ala-Pro-Arg-Thr-Leu-Cys-Leu-COOH |
| 250 | NH2-Val-Met-Ala-Pro-Arg-Thr-Leu-Phe-Cys-COOH |
| 251 | NH2-Val-Met-Cys-Pro-Arg-Thr-Leu-Cys-Leu-COOH |
| 252 | NH2-hAr-Nle-Lys(N3)-Gln-Mff-Ala-Cha-hAr-Leu-COOH |
| 253 | NH2-hAr-Nle-hAr-Gln-Mff-Ala-Cha-Lys(N3)-Leu-COOH |
| 254 | NH2-Cha-hAr-Ala-Leu-hAr-Gln-Nle-Cys-hAr-COOH |
| 255 | NH2-Asn-Ala-Leu-Asp-Glu-Tyr-Cys-Glu-Asp-Lys-Asn-Arg-CONH2 |
| 256 | NH2-Asn-Ala-Leu-Asp-Glu-Tyr-Lys(PEG2-FITC)-Glu-Asp-Lys-Asn-Arg-CONH2 |
| 257 | NH2-Cha-Cys-Ala-Leu-hAr-Gln-Nle-Mff-hAr-COOH |
| 258 | NH2-hAr-Nle-Cys-Gln-Mff-Ala-Cha-hAr-Leu-COOH |
| 259 | NH2-Val-Met-Ala-Pro-Lys(PEG2-DOTA)-Thr-Leu-Cys-Leu-COOH |
| 260 | NH2-Val-Met-Ala-Pro-Lys(PEG2-FITC)-Thr-Leu-Cys-Leu-COOH |
| 261 | NH2-Val-Met-Cys-Pro-Lys(FITC)-Thr-Leu-Phe-Leu-COOH |
| 262 | NH2-Val-Met-Cys-Pro-Lys(DOTA)-Thr-Leu-Phe-Leu-COOH |
| 263 | NH2-Val-Met-Cys-Pro-Lys(PEG4-biotin)-Thr-Leu-Phe-Leu-COOH |
| 264 | NH2-Val-Met-Cys-Pro-Lys(PEG2-ibu)-Thr-Leu-Phe-Leu-COOH |
| 265 | NH2-Val-Met-Ala-Pro-Lys(PEG2-ibu)-Thr-Leu-Cys-Leu-COOH |
| 266 | NH2-Val-Met-Ala-Pro-Lys(PEG4-biotin)-Thr-Leu-Cys-Leu-COOH |
| 267 | NH2-hAr-Nle-hAr-Gln(13C5,15N2)-Mff-Ala-Cha-hAr-Leu-COOH |
| 268 | NH2-Val-Met-Cys(mSF)-Pro-Lys(FITC)-Thr-Leu-Phe-Leu-COOH |
| 269 | NH2-Val-Met-Ala-Pro-Lys(PEG5-Lys(GalNAc)-Lys(GalNAc)-Lys(GalNAc))- |
| | Thr-Leu-Cys-Leu-COOH |
| 270 | NH2-Bhp-Val-Asn-Arg-Leu-Lys-COOH |
| 271 | NH2-Bhp-Ser-Gln-Gln-Phe-COOH |
| 272 | NH2-Val-Met-Ala-Pro-Lys(Tz-CD94[111-114])-Thr-Leu-Cys-Leu-COOH |
| 273 | NH2-Val-Met-Lys(Tz-NKG2A[213-217])-Pro-Arg-Thr-Leu-Cys-Leu-COOH |
| 274 | NH2-Val-Met-Ala-Pro-Bhp-Thr-Leu-Cys-Leu-COOH |
| 275 | NH2-NKG2A[123-232]_130Ser_163Nle_171Pro(N3)_189Nle; UniProt P26715- |
| | CONH2 |
| 276 | NH2-Val-Met-Ala-Pro-Bhp(Tz-NKG2A[123-232]_ |
| | 130Ser_163Nle_171Pro(Tz)_189Nle)-Thr-Leu-Cys-Leu-COOH |
| 277 | NH2-Val-Met-Ala-Pro-Bhp(Tz-NKG2A[123-232]_ |
| | 130Ser_163Nle_171Pro(Tz)_189Nle)-Thr-Leu-Cys-Leu-COOH |
| 278 | NH2-Ser-Phe-Cys-Asn-Ile-Ser-Gly-Gly-Ile-His-Cys-CONH2 |
| 279 | NH2-Ser-Trp-Cys-Thr-Lys-Thr-Asn-Asp-Gln-Arg-Cys-CONH2 |
| 280 | NH2-Ser-His-Cys-Ser-Asp-Thr-Gln-Glu-Ala-Tyr-Cys-CONH2 |
| 281 | NH2-Ser-Pro-Cys-Glu-Arg-Trp-Ala-Ile-Ser-Phe-Cys-CONH2 |
| 282 | NH2-Ser-Ile-Cys-His-Gly-Ile-Glu-Tyr-Pro-Cys-CONH2 |
| 283 | NH2-Val-Nle-Ala-Pro-Dap(Hya)-Thr-Leu-Phe-Leu-COOH |
| 284 | NH2-Ser-Phe-Cys(ox.)-Asn-Ile-Ser-Gly-Gly-Ile-His-Cys(ox.)-CONH2 |
| 285 | NH2-Ser-Trp-Cys(ox.)-Thr-Lys-Thr-Asn-Asp-Gln-Arg-Cys(ox.)-CONH2 |
| 286 | NH2-Ser-His-Cys(ox.)-Ser-Asp-Thr-Gln-Glu-Ala-Tyr-Cys(ox.)-CONH2 |
| 287 | NH2-Ser-Pro-Cys(ox.)-Glu-Arg-Trp-Ala-Ile-Ser-Phe-Cys(ox.)-CONH2 |
| 288 | NH2-Ser-Ile-Cys(ox.)-His-Gly-Ile-Glu-Tyr-Pro-Cys(ox.)-CONH2 |
| 289 | NH2-Leu-Cys-Thr-Leu-Val-Pro-Met-Ala-Arg-COOH |
| 290 | NH2-Leu-Phe-Thr-Leu-Val-Pro-Met-Ala-Arg-COOH |
| 291 | NH2-Leu-Cys(mSF)-Thr-Leu-Val-Pro-Met-Ala-Arg-COOH |
| 292 | DMBA-Met-Ala-Pro-Arg-Thr-Leu-Cys-Leu-COOH |
| 293 | HMPA-Met-Ala-Pro-Arg-Thr-Leu-Cys-Leu-COOH |
| 294 | NMeH-Val-Met-Ala-Pro-Arg-Thr-Leu-Cys-Leu-COOH |
| 295 | NH2-Val-Met-Ala-Pro-Arg-Thr-Leu-Dab-Leu-COOH |
| 296 | NH2-Val-Met-Ala-Pro-Arg-Thr-Leu-Dap-Leu-COOH |
| 297 | HMBA-Met-Ala-Pro-Arg-Thr-Leu-Cys-Leu-COOH |
| 298 | NH2-Val-Met-Ala-Pro-Cys(FAM)-Thr-Leu-Phe-Leu-COOH |
| 299 | NH2-Val-Nle-Ala-Pro-Arg-Thr-Leu-Cys-Leu-COOH |
| 300 | NH2-Val-Nle-Ala-Pro-Arg-Thr-Leu-hCys-Leu-COOH |
| 301 | NH2-Val-Nle-Ala-Pro-Pgl-Thr-Leu-Cys-Leu-COOH |
| 302 | NH2-Val-Nle-Ala-Pro-Bhp-Thr-Leu-Cys-Leu-COOH |
| 303 | NH2-Pro-Arg-Leu-Met-Ala-Leu-Val-Cys-Thr-COOH |
| 304 | NH2-Pro-Arg-Leu-Met-Ala-Leu-Val-Cys(mSF)-Thr-COOH |
| 305 | NH2-Pro-Arg-Leu-Met-Cys-Leu-Val-Phe-Thr-COOH |
| 306 | NH2-Pro-Arg-Leu-Met-Cys(FAM)-Leu-Val-Phe-Thr-COOH |
| 307 | NH2-Val-Met-Ala-Pro-Ala-Thr-Leu-Cys-Leu-COOH |
| 308 | NH2-Val-Met-Ala-Pro-Lys-Thr-Leu-Cys-Leu-COOH |
| 309 | NH2-Val-Met-Ala-Pro-hArg-Thr-Leu-Cys-Leu-COOH |
| 310 | NH2-Val-Met-Ala-Pro-Cav-Thr-Leu-Cys-Leu-COOH |
| 311 | NH2-Val-Met-Ala-Pro-DArg-Thr-Leu-Cys-Leu-COOH |
| 312 | NH2-Val-Met-Ala-Pro-Arg-Thr-Leu-Cys-Ala-COOH |
| 313 | NH2-Val-Met-Ala-Pro-Arg-Thr-Ala-Cys-Leu-COOH |
| 314 | NH2-Val-Met-Ala-Pro-Arg-Ala-Leu-Cys-Leu-COOH |
| 315 | NH2-Val-Met-Ala-Pro-Ala-Thr-Leu-Cys-Leu-COOH |
| 316 | NH2-Val-Met-Ala-Ala-Arg-Thr-Leu-Cys-Leu-COOH |
| 317 | NH2-Val-Met-Gly-Pro-Arg-Thr-Leu-Cys-Leu-COOH |
| 318 | NH2-Val-Ala-Ala-Pro-Arg-Thr-Leu-Cys-Leu-COOH |
| 319 | NH2-Ala-Met-Ala-Pro-Arg-Thr-Leu-Cys-Leu-COOH |
| 320 | NH2-Ala-Leu-Cys-Ser-Arg-Val-Pro-Cys-Ser-Gly-Gly-Lys(Bio)-CONH2 |
| 321 | NH2-Ala-Leu-Cys-Glu-Pro-Ser-Leu-Cys-Leu-Gly-Gly-Lys(Bio)-CONH2 |
| 322 | NH2-Ala-Leu-Cys-Gly-Arg-Ser-Cys-Leu-Val-Gly-Gly-Lys(Bio)-CONH2 |
| 323 | NH2-Ala-Leu-Cys-Gly-Leu-Ser-Cys-Leu-Val-Gly-Gly-Lys(Bio)-CONH2 |
| 324 | NH2-Ala-Leu-Cys-Gly-Ala-Ser-Cys-Leu-Phe-Gly-Gly-Lys(Bio)-CONH2 |
| 325 | NH2-Ala-Leu-Cys-Gly-Ser-Arg-Cys-Leu-Phe-Gly-Gly-Lys(Bio)-CONH2 |
| 326 | NH2-Ala-Leu-Cys-Gly-Ser-Ala-Cys-Leu-Tyr-Gly-Gly-Lys(Bio)-CONH2 |
| 327 | NH2-Ala-Leu-Cys-Gly-Arg-His-Cys-Leu-Phe-Gly-Gly-Lys(Bio)-CONH2 |
| 328 | NH2-Ala-Leu-Cys-Gly-Arg-Thr-Cys-Leu-Thr-Gly-Gly-Lys(Bio)-CONH2 |
| 329 | NH2-Ala-Leu-Cys-Gly-Arg-Ala-Cys-Leu-Trp-Gly-Gly-Lys(Bio)-CONH2 |
| 330 | NH2-Ala-Met-Cys-Gly-Arg-Leu-Cys-Leu-Phe-Gly-Gly-Lys(Bio)-CONH2 |
| 331 | NH2-Ala-Met-Cys-Gly-Leu-Pro-Cys-Leu-Val-Gly-Gly-Lys(Bio)-CONH2 |
| 332 | NH2-DhAr-Nle-hAr-Gln-Mff-Ala(NMe)-Cha-Cys(mSF)-Leu-COOH |
| 333 | NH2-DhAr-Nle-hAr-Gln-Mff-Ala-Cha-Cys(mSF)-Leu-COOH |
| 334 | NH2-Ala-Leu-Cys-Trp-Arg-Val-Pro-Cys-Thr-Gly-Gly-Lys(FITC)-CONH2 |
| 335 | NH2-Ala-Met-Cys-Pro-Pro-Trp-Met-Cys-Phe-Gly-Gly-Lys(FITC)-CONH2 |
| 336 | NH2-Ala-Met-Cys-Gly-Ala-Arg-Cys-Leu-Phe-Gly-Gly-Lys(FITC)-CONH2 |
| 337 | NH2-Ala-Nle-Cys-Gly-Pro-Arg-Cys-Leu-Trp-Gly-Gly-Lys(FITC)-CONH2 |
| 338 | NH2-Met-Cys-Gly-Pro-Arg-Cys-Leu-Trp-Gly-Gly-Lys(FITC)-CONH2 |
| 339 | NH2-Ala-Leu-Cys-Trp-Arg-Val-Ser-Cys-Thr-Gly-Gly-Lys(FITC)-CONH2 |
| 340 | NH2-Gly-Met-Cys-Gly-Pro-Arg-Cys-Leu-Trp-Gly-Gly-Lys(FITC)-CONH2 |
| 341 | NH2-Ala-Ala-Cys-Gly-Pro-Arg-Cys-Leu-Trp-Gly-Gly-Lys(FITC)-CONH2 |
| 342 | NH2-Ala-Met-Cys-Ala-Pro-Arg-Cys-Leu-Trp-Gly-Gly-Lys(FITC)-CONH2 |
| 343 | NH2-Ala-Met-Cys-Gly-Ala-Arg-Cys-Leu-Trp-Gly-Gly-Lys(FITC)-CONH2 |
| 344 | NH2-Ala-Met-Cys-Gly-Pro-Ala-Cys-Leu-Trp-Gly-Gly-Lys(FITC)-CONH2 |
| 345 | NH2-Ala-Met-Cys-Gly-Pro-Arg-Cys-Ala-Trp-Gly-Gly-Lys(FITC)-CONH2 |
| 346 | NH2-Ala-Met-Cys-Gly-Pro-Arg-Cys-Leu-Ala-Gly-Gly-Lys(FITC)-CONH2 |
| 347 | NH2-Gln-Gln-Phe-DPro-Gly-Asn-Pro-Asn-Gly-Asn-Ala-Leu-Asp-Glu-Ser-Abu- |
| | Lys(biotin)-CONH2 |
| 348 | NH2-Gln-Gln-Phe-DPro-Pro-Asn-Pro-Asn-Gly-Asn-Ala-Leu-Asp-Glu-Ser-Abu- |
| | Lys(biotin)-CONH2 |
| 349 | NH2-Gln-Gln-Phe-Ala-Aib-Asn-Pro-Asn-Gly-Asn-Ala-Leu-Asp-Glu-Ser-Abu- |
| | Lys(biotin)-CONH2 |
| 350 | NH2-Cys(mSF)-Nle-hAr-Gln-Mff-Ala-Cha-hAr-Leu-COOH |
| 351 | NH2-Cys(pSF)-Nle-hAr-Gln-Mff-Ala-Cha-hAr-Leu-COOH |
| 352 | NH2-Cha-hAr-Ala-Leu-hAr-Gln-Nle-Mff-hAr-COOH |
| 353 | NH2-Val-Met-Ala-Pro-Arg-Thr-Leu-Cys(mOSF)-Leu-COOH |
| 354 | NH2-Val-Met-Ala-Pro-Ala-Thr-Leu-Phe-Leu-Gly-Gly-Lys(FITC)-CONH2 |
| 355 | AMAPRTLLL |

What is claimed is:

1. A synthetic peptide comprising an amino acid sequence X-Met-X-X-Z-Ala-X-U-Leu (SEQ ID NO: 3), wherein X is 4Af, hAr, Ala, Dff, Ser, Msn, Gln, Cha, or Arg; Z is Ala, Cha, Tha, or Mff; and U is Arg, Msn, or hAR.

2. The synthetic peptide of claim 1, wherein the amino acid sequence is
(a) NH2-4Af-Met-Dff-Ser-Ala-Ala-Cha-Arg-Leu-OH (SEQ ID NO: 5);
(b) NH2-hAr-Met-Msn-Dff-Cha-Ala-Arg-Msn-Leu-OH (SEQ ID NO: 6);
(c) NH2-Ala-Met-hAr-Dff-Tha-Ala-Cha-Arg-Leu-OH (SEQ ID NO: 7);
(d) NH2-4Af-Met-Ala-Ser-Ala-Ala-Cha-Arg-Leu-OH (SEQ ID NO: 8); or
(e) NH2-hAR-Met-hAR-Gln-Mff-Ala-Cha-hAR-Leu-OH (SEQ ID NO: 9).

3. The synthetic peptide of claim 1 or claim 2, wherein one or more amino acids is substituted with a Cys, Lys, Tyr, His, Ser, or Thr.

4. The synthetic peptide of claim 3, wherein the substituted amino acid is at position 3 or position 8.

5. The synthetic peptide of claim 4, wherein the substituted amino acid is at position 8 and is substituted with a Cys.

6. The synthetic peptide of claim 4, wherein the substituted amino acid is at position 3 and is substituted with a Cys.

7. The synthetic peptide of any one of claims 3 to 6, wherein the one or more Cys, Lys, Tyr, His, Ser, or Thr is arylated.

8. The synthetic peptide of any one of claims 3 to 6, wherein the one or more Cys, Lys, Tyr, His, Ser, or Thr is conjugated to a warhead.

9. The synthetic peptide of claim 8, wherein the warhead is 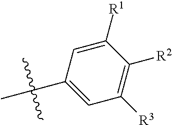
wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

10. The synthetic peptide of claim 8, wherein the warhead is

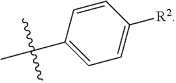

11. The synthetic peptide of claim 8, wherein the warhead is

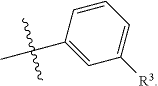

12. The synthetic peptide of claim 8, wherein the warhead is 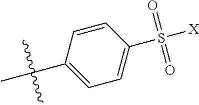
wherein R¹⁰ is hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

13. The synthetic peptide of claim 8, wherein the warhead is 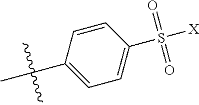
wherein X is a halogen.

14. The synthetic peptide of claim 8, wherein the warhead is

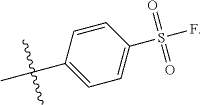 [SMILES: *c1ccc(S(=O)(=O)F)cc1]

15. The synthetic peptide of claim 8, wherein the warhead is selected from the group consisting of

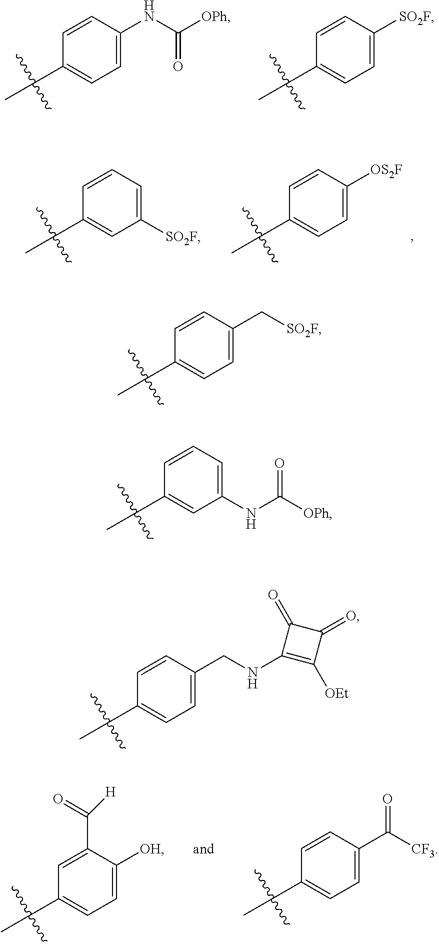

16. The synthetic peptide of claim 8, wherein the warhead is selected from the group of a sulfonyl fluoride, a phenyl carbamate, and a squareamate.

17. The synthetic peptide any one of claims 8 to 16, wherein the warhead is conjugated to the Cys via the Sulfur atom of the Cys.

18. A synthetic peptide comprising an amino acid sequence hAr-X-hAr-Gln-Mff-A-Cha-hAr-Z (SEQ ID NO: 20) wherein X is Nle or Mox; and Z is Let, Aoa, or Cha.

19. The synthetic peptide of claim 18, wherein the amino acid sequence is
(a) NH2-hAr-Nle-hAr-Gln-Mff-Aa-Cha-hAr-Leu-OH (SEQ ID NO: 21);
(b) NH2-hAr-Mox-hAr-Gin-Mff-Ala-Cha-hAr-Leu-OH (SEQ ID NO: 22);
(c) NH2-hAr-Met-hAr-Gln-Mff-Ala-Cha-hAr-Nle-OH (SEQ ID NO: 23);
(d) NH2-hAr-Nle-hAr-Gin-Mff-Ala-Cha-hAr-Aoa-OH (SEQ ID NO: 24); or
(e) NH2-hAr-Nle-hAr-Gln-Mff-Ala-Cha-hAr-Cha-H (SEQ ID NO: 25).

20. The synthetic peptide off claim 18 or claim 19, wherein one or more amino acids is substituted with a Cys, Lys, Tyr, His, Ser, or Thr.

21. The synthetic peptide of any one of claims 18 to 20, wherein the substituted amino acid is at position 3 or position 8.

22. The synthetic peptide of claim 21, wherein the substituted amino acid is at position 8 and is substituted with a Cys.

23. A synthetic peptide comprising the formula

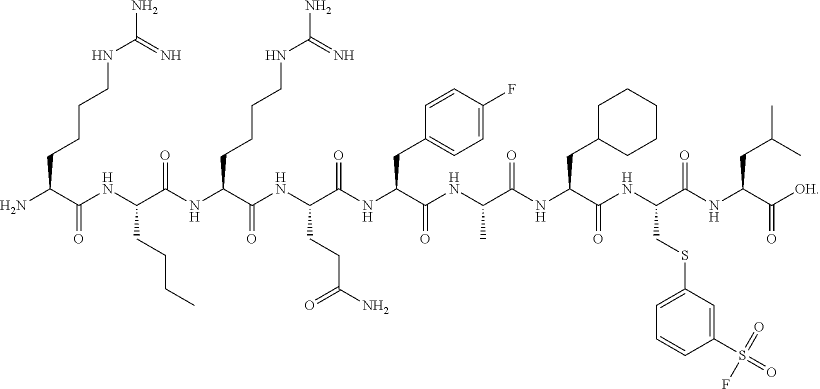 [SMILES: CCCC[C@H](NC(=O)[C@@H](N)CCCCNC(=N)N)C(=O)N[C@@H](CCCCNC(=N)N)C(=O)N[C@@H](CCC(N)=O)C(=O)N[C@@H](Cc1ccc(F)cc1)C(=O)N[C@@H](C)C(=O)N[C@@H](CC1CCCCC1)C(=O)N[C@@H](CSc1cccc(S(=O)(=O)F)c1)C(=O)N[C@@H](CC(C)C)C(=O)O]

24. A synthetic peptide comprising the formula 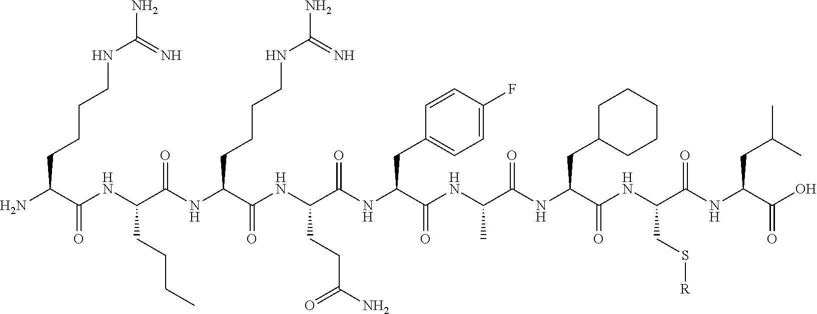
wherein the R is 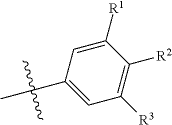
wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

25. A synthetic peptide comprising an amino acid sequence VMAPRT(L/V)(V/L/I/F)L wherein one or more amino acids are substituted with a Cys, Lys, Tyr, His, Ser, or Thr.

26. The synthetic peptide of claim 25, wherein the substituted amino acid is at position 3 or position 8.

27. The synthetic peptide of claim 26, wherein the substituted amino acid is at position 8 and is substituted with a Cysteine.

28. The synthetic peptide of any one of claims 25 to 27, wherein the peptide has the amino acid sequence VMAPRTLFL.

29. The synthetic peptide of any one of claims 25 to 28 wherein the one or more Cys, Lys, Tyr, His, Ser, or Thr is conjugated to a warhead.

30. The synthetic peptide of claim 29, wherein the warhead is 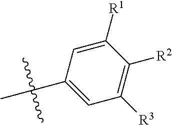
wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

31. The synthetic peptide of claim 29, wherein the warhead is

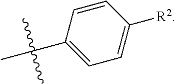

32. The synthetic peptide of claim 29, wherein the warhead is

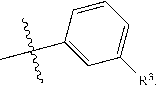

33. The synthetic peptide of claim 29, wherein the warhead is 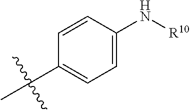
wherein R¹⁰ is hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

34. The synthetic peptide of claim 29, wherein the warhead is 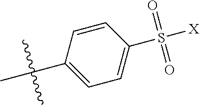
wherein X is a halogen.

35. The synthetic peptide of claim 29, wherein the warhead is

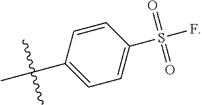 [SMILES: *c1ccc(S(=O)(=O)F)cc1]

36. The synthetic peptide of claim 29, wherein the warhead is selected from the group consisting of

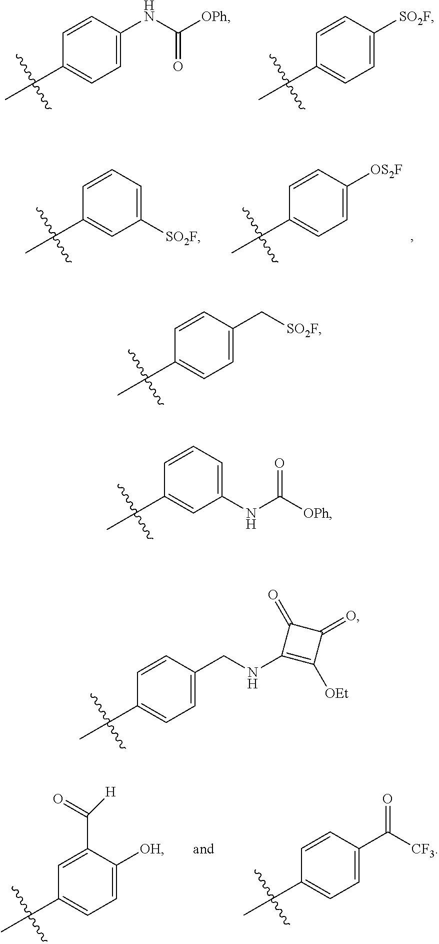

37. The synthetic peptide of any one of claims 29 to 36, wherein the warhead is conjugated to the Cys via the Sulfur atom of the Cys.

38. A synthetic peptide comprising the formula

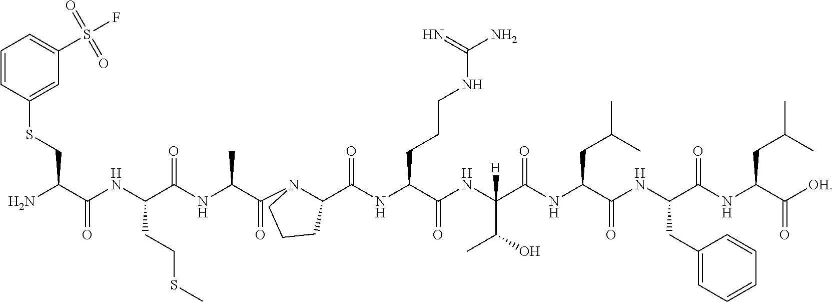 [SMILES: CSCC[C@H](NC(=O)[C@@H](N)CSc1cccc(S(=O)(=O)F)c1)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

39. A synthetic peptide comprising the formula

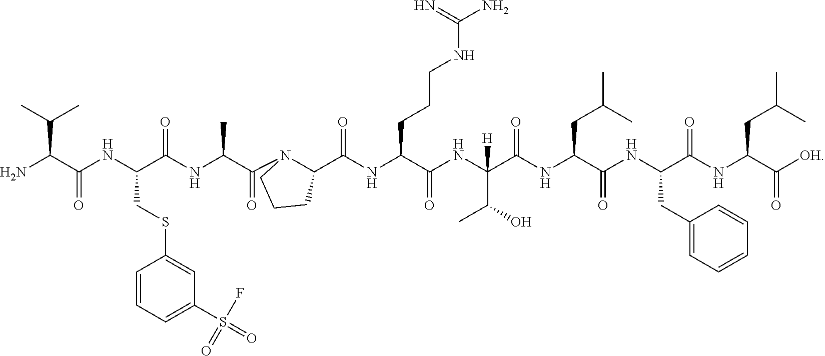 [SMILES: CC(C)C[C@H](NC(=O)[C@H](Cc1ccccc1)NC(=O)[C@H](CC(C)C)NC(=O)[C@@H](NC(=O)[C@H](CCCNC(=N)N)NC(=O)[C@@H]1CCCN1C(=O)[C@H](C)NC(=O)[C@H](CSc1cccc(S(=O)(=O)F)c1)NC(=O)[C@@H](N)C(C)C)[C@@H](C)O)C(=O)O]

40. A synthetic peptide comprising the formula

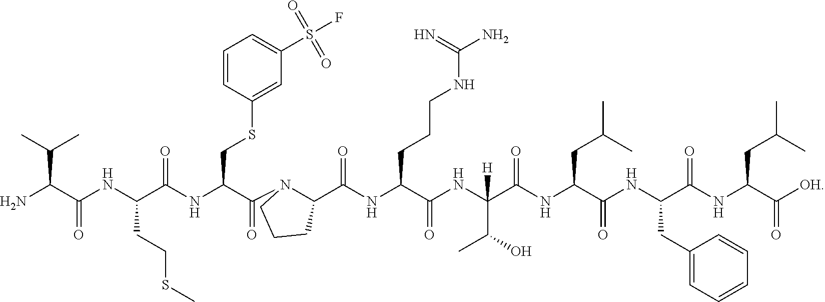 [SMILES: CSCC[C@H](NC(=O)[C@@H](N)C(C)C)C(=O)N[C@@H](CSc1cccc(S(=O)(=O)F)c1)C(=O)N1CCC[C@H]1C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

41. A synthetic peptide comprising the formula

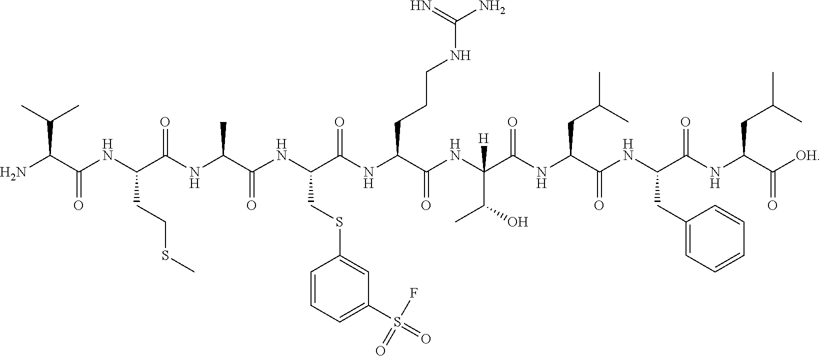 [SMILES: CSCC[C@H](NC(=O)[C@@H](N)C(C)C)C(=O)N[C@@H](C)C(=O)N[C@@H](CSc1cccc(S(=O)(=O)F)c1)C(=O)N[C@@H](CCCNC(=N)N)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

42. A synthetic peptide comprising the formula

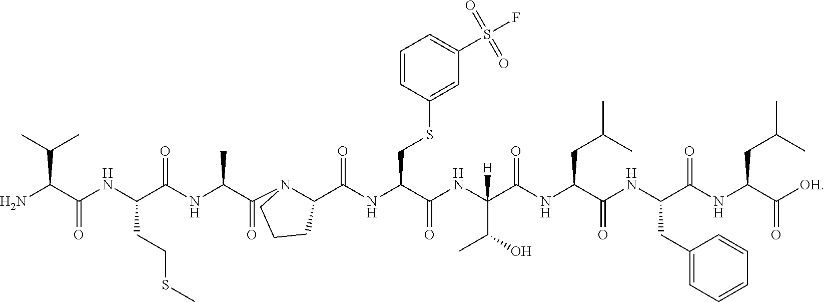 [SMILES: CSCC[C@H](NC(=O)[C@@H](N)C(C)C)C(=O)N[C@@H](C)C(=O)N1CCC[C@H]1C(=O)N[C@@H](CSc1cccc(S(=O)(=O)F)c1)C(=O)N[C@H](C(=O)N[C@@H](CC(C)C)C(=O)N[C@@H](Cc1ccccc1)C(=O)N[C@@H](CC(C)C)C(=O)O)[C@@H](C)O]

43. A synthetic peptide comprising the formula

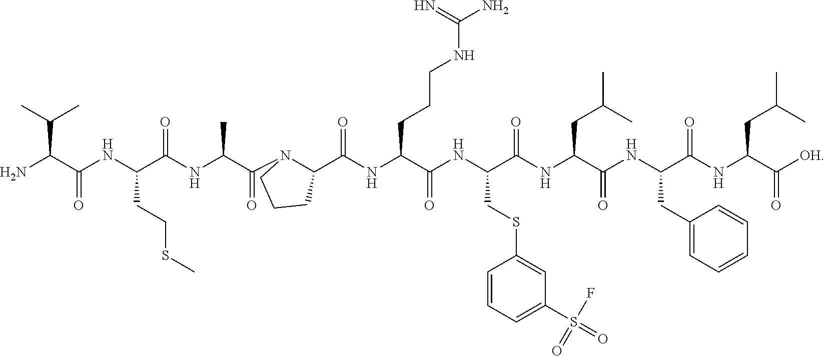 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@@H](CSc1cccc(S(=O)(=O)F)c1)C(=O)N[C@@H](CC(C)C)C(=O)N[C@@H](Cc1ccccc1)C(=O)N[C@@H](CC(C)C)C(=O)O]

44. A synthetic peptide comprising the formula

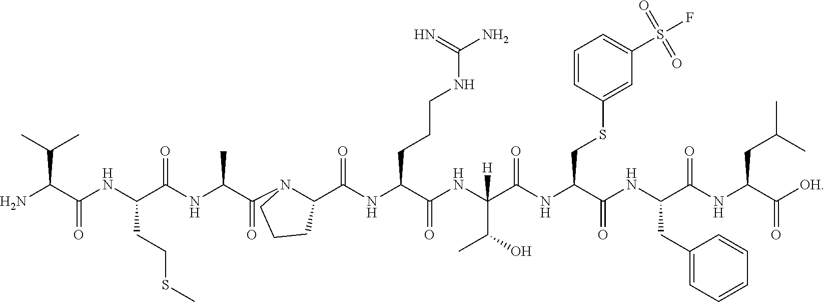 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CSc1cccc(S(=O)(=O)F)c1)C(=O)N[C@@H](Cc1ccccc1)C(=O)N[C@@H](CC(C)C)C(=O)O)[C@@H](C)O]

45. A synthetic peptide comprising the formula

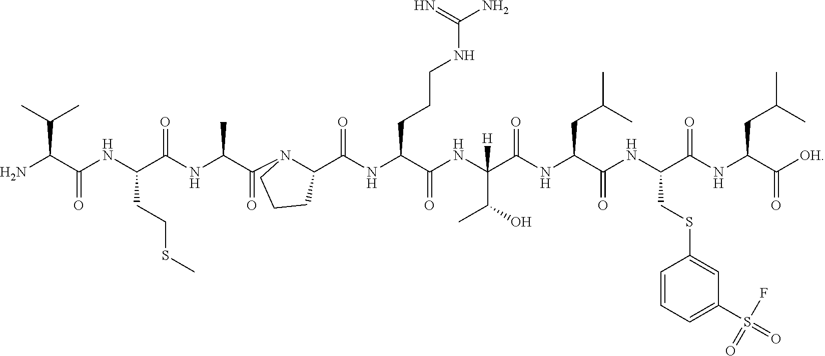 [SMILES: CSCC[C@H](NC(=O)[C@@H](N)C(C)C)C(=O)N[C@@H](C)C(=O)N1CCC[C@H]1C(=O)N[C@@H](CCCNC(=N)N)C(=O)N[C@H](C(=O)N[C@@H](CC(C)C)C(=O)N[C@@H](CSc1cccc(S(=O)(=O)F)c1)C(=O)N[C@@H](CC(C)C)C(=O)O)[C@@H](C)O]

46. A synthetic peptide comprising the formula

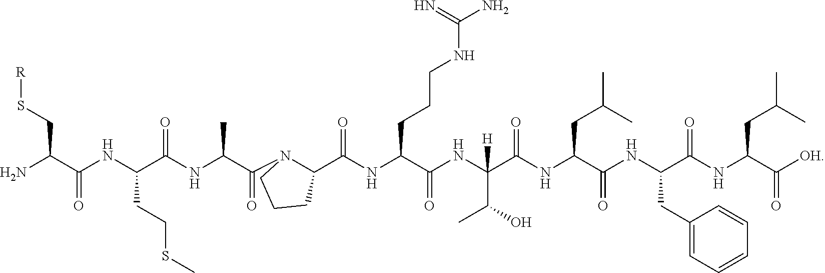

47. A synthetic peptide comprising the formula

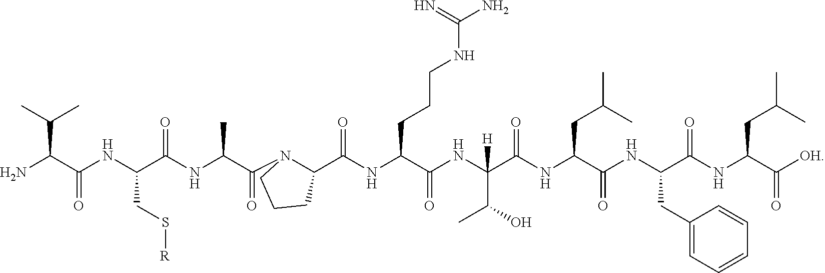

48. A synthetic peptide comprising the formula

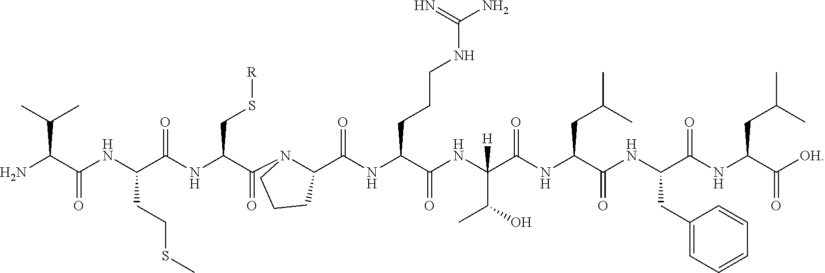

49. A synthetic peptide comprising the formula

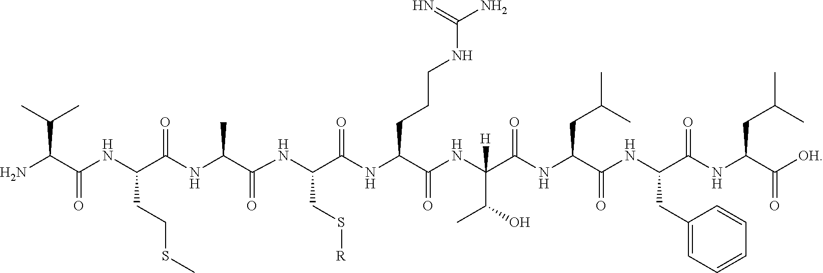

50. A synthetic peptide comprising the formula

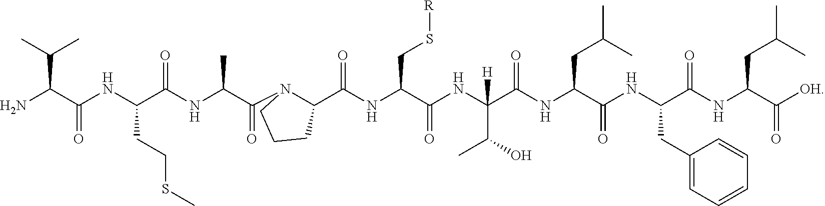

51. A synthetic peptide comprising the formula

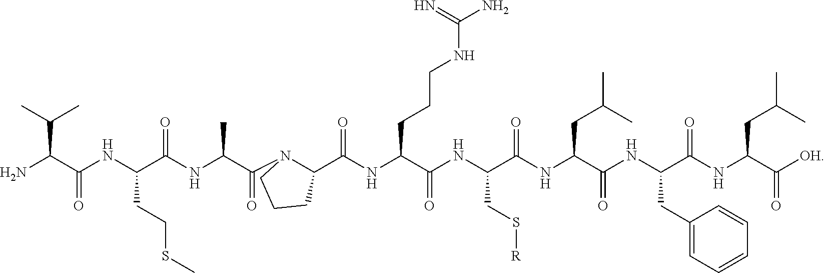

52. A synthetic peptide comprising the formula

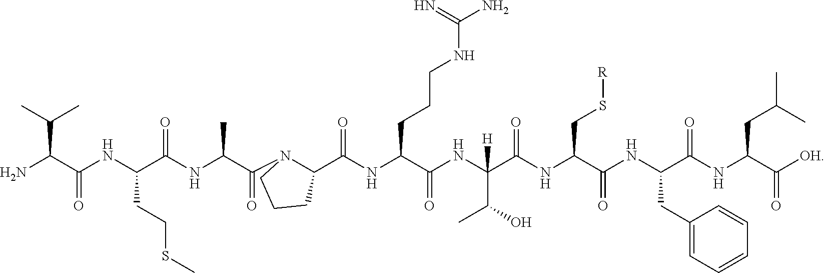

53. A synthetic peptide comprising the formula

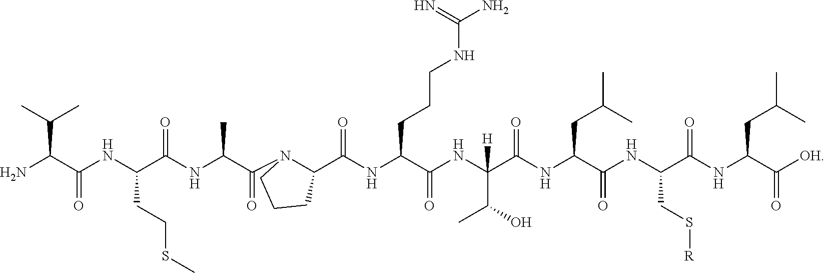

54. The synthetic peptide of any one of claims 46 to 53, wherein R is 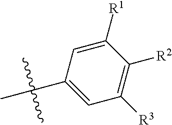
wherein R¹, R², and R³ are each independently hydrogen, halogen, —CN, —NO₂, substituted or unsubstituted alkyl, substituted or unsubstituted alkenyl, substituted or unsubstituted alkynyl, —OR^{C3}, —C₁-C₆ alkyl N(R^{C3})₂, —N(R^{C3})₂, —SR^{C3}, —C(═O)R^{C3}, —C(═O)OR^{C3}, —C(═O)SR^{C3}, —C(═O)N(R^{C3})₂, —OC(═O)R^{C3}, —OC(═O)OR^{C3}, —OC(═O)N(R^{C3})₂, —OC(═O)SR^{C3}, —OS(═O)₂R^{C3}, —C₁-C₆ alkyl OS(═O)₂R^{C3}, —OS(═O)₂OR^{C3}, —OS(═O)₂N(R^{C3})₂, —N(R^{C3})C(═O)R^{C3}, —N(R^{C3})C(═NR^{C3})R^{C3}, —N(R^{C3})C(═O)OR^{C3}, —N(R^{C3})C(═O)N(R^{C3})₂, —N(R^{C3})C(═NR^{C3}) N(R^{C3})₂, —N(R^{C3})S(═O)₂R^{C3}, —N(R^{C3})S(═O)₂OR^{C3}, —N(R^{C3})S(═O)₂N(R^{C3})₂, —SC(═O)R^{C3}, —SC(═O)OR^{C3}, —SC(═O)SR^{C3}, —SC(═O)N(R^{C3})₂, —S(═O)₂R^{C3}, —S(═O)₂OR^{C3}, or —S(═O)₂N(R^{C3})₂, wherein each instance of R^{C3} is independently selected from hydrogen, OPh, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted carbocyclyl, or substituted or unsubstituted heterocyclyl.

55. The synthetic peptide of any one of claims 1 to 54, wherein the synthetic peptide is an HLA-E-NKG2A complex specific inhibitor.

56. A synthetic peptide comprising the amino acid sequence of VMAPRTLFL with one or more amino acid substitution.

57. The synthetic peptide of claim 56, wherein the peptide comprises:
a) a substitution of the V residue at a position 1;
b) a substitution of the M residue at a position 2;
c) a substitution of the A residue at a position 3;
d) a substitution of the P residue at a position 4;
e) a substitution of the R residue at a position 5;
f) a substitution of the T residue at a position 6;
g) a substitution of the L residue at a position 7;
h) a substitution of the F residue at a position 8;
i) a substitution of the L residue at a position 9;
or a combination of any of the foregoing substitutions.

58. The synthetic peptide of any one of claims 56 or 57, wherein the substitution is at position 1 and the amino acid is substituted for a 4Af, hAr, Ala, Dff, Ser, Msn, Gln, Cia, or Arg.

59. The synthetic peptide of any one of claims 56 or 57, wherein the substitution is at position 3 and the amino acid is substituted for a 4Af, hAr, Ala, Dff, Ser, Msn, Gln, Cha, or Arg.

60. The synthetic peptide of any one of claims 56 or 57, wherein the substitution is at position 4 and the amino acid is substituted for a 4Af, hAr, Ala, Dff, Ser, Msn, Gln, Cha, or Arg.

61. The synthetic peptide of any one of claims 56 or 57, wherein the substitution is at position 5 and the amino acid is substituted for an Ala, Cha, Tha, or Mff.

62. The synthetic peptide of any one of claims 56 or 57, wherein the substitution is at position 7 and the amino acid is substituted for a 4Af, hAr, Ala, Dff, Ser, Msn, Gln, Cha, or Arg.

63. The synthetic peptide of any one of claims 56 or 57, wherein the substitution is at position 8 and the amino acid is substituted for a Arg, Msn, or hAR.

64. The synthetic peptide of any one of claims 56 or 57, wherein the substitution is at position 3 and the amino acid is substituted for a Cys, Lys, Tyr, His, Ser, or Thr.

65. The synthetic peptide of any one of claims 56 or 57, wherein the substitution is at position 8 and the amino acid is substituted for a Cys, Lys, Tyr, His, Ser, or Thr.

66. A synthetic peptide of any one of claims 1-65 comprising one or more additional modifications selected from:
a) acetylated, formylated, propanoylated, hexanoylated, or myristoylated N-terminus;
b) amidated C-terminus;
c) substitution of one or more L-amino acid with a D-amino acid;
d) substitution of one or more amino acid with a methyl-amino acid; and
e) substitution of an α-amino acid with a β-amino acids.

67. A synthetic peptide/HLA-E complex, wherein the peptide is selected from any one of the synthetic peptides of claims 1 to 66.

68. The complex of claim 67, wherein the synthetic peptide and the HLA-E in the complex are covalently linked.

69. The complex of any one of claims 67 or 68, wherein the HLA-E is human HLA-E.

70. The complex of any one of claims 67 to 69, wherein the synthetic peptide is covalently linked to amino acid residue Tyr-7, Lys-146, Tyr-159, or Tyr-171 of human 1HLA-E.

71. The complex of any one of claims 67 to 70, wherein the synthetic peptide is covalently linked to an amino acid residue selected from the group of Tyr-7, His-9, Ser-24, Tyr-59, Arg-62, Glu-63, Ser-66, Thr-70, Gln-72, Asn-77, Thr-80, Tyr-84, Trp-97, His-99, Glu-114, Tyr-123, Trp-133, Ser-143, Lys-146, Ser-147, Glu-152, His-155, Gln-156, Tyr-159, Thr-163, Cys-164, Trp-167, and Tyr-171 of human HLA-E.

72. The HLA-E peptide complex of any one of claims 67 to 71, wherein the complex is inhibited in binding of CD94/NKG2A or prevents activation of CD94/NKG2A.

73. A synthetic peptide/HLA-E complex, wherein the synthetic peptide is covalently linked to amino acid residue Tyr-7, Tyr-171, Tyr-159, or Lys-146 of human HLA-E.

74. A pharmaceutical composition, comprising the synthetic peptide of any one of claims 1-73 and a pharmaceutically acceptable salt or carrier.


SEQUENCE LISTING (419 sequences)

SEQ ID NO: 1 - protein, 9 aa, synthetic construct
  VARIANT 7: note = X can be L or V
  VARIANT 8: note = X can be V,L,I or F
VMAPRTXXL

SEQ ID NO: 2 - protein, 9 aa, synthetic construct
VMAPRTLVL

SEQ ID NO: 3 - skipped

SEQ ID NO: 4 - protein, 10 aa, synthetic construct
  VARIANT 1: note = X can be G,A,M,P,S,N,Q,F,Y,H,W,D,E,K.R,Beta-cyclopr opyl-L-Ala-OH,Beta-cyclohexyl-L-Ala-OH,L-Met sulfone-OH,3- (4-prime-pyridyl)-L-Ala-OH,4-(Boc-amino)-L-Phe-OH,3-(4-thi azolyl)-L-Ala-OH,3,4-difluoro-L-Phe-OH,N-omega-(Pbf)-L-hom oArg-OH or L-alpha-aminoadipic acid(delta tBu)
  VARIANT 3..4: note = X can be G,A,M,P,S,N,Q,F,Y,H,W,D,E,K.R,Beta-cyclopr opyl-L-Ala-OH,Beta-cyclohexyl-L-Ala-OH,L-Met sulfone-OH,3- (4-prime-pyridyl)-L-Ala-OH,4-(Boc-amino)-L-Phe-OH,3-(4-thi azolyl)-L-Ala-OH,3,4-difluoro-L-Phe-OH,N-omega-(Pbf)-L-hom oArg-OH or L-alpha-aminoadipic acid(delta tBu)
  VARIANT 5: note = X can be G,A,V,L,M,P,F,W,Beta-cyclopropyl-L-Ala-OH, Beta-cyclobutyl-L-Ala-OH,Beta-cyclohexyl-L-Ala-OH,L-2-amin ooctanoic acid,4-fluoro-L-phenylalanine,3,4-difluoro-L-Phe -OH,3,4,5-trifluoro-L-Phe-OH,3-(4-thiazolyl)-L-Ala-OH,3-(2 -naphtyl)-L-Ala-OH,HomoPhe-OH (hPh),3,4-dimethoxy-L-Phe-OH ,4-phenylpiperidine-4-carboxylic acid or (4-aminomethyl) benzoic acid
  VARIANT 7: note = X can be G,A,M,P,S,N,Q,F,Y,H,W,D,E,K.R,Beta-cyclopr opyl-L-Ala-OH,Beta-cyclohexyl-L-Ala-OH,L-Met sulfone-OH,3- (4-prime-pyridyl)-L-Ala-OH,4-(Boc-amino)-L-Phe-OH,3-(4-thi azolyl)-L-Ala-OH,3,4-difluoro-L-Phe-OH,N-omega-(Pbf)-L-hom oArg-OH or L-alpha-aminoadipic acid(delta tBu)
  VARIANT 8: note = X can be S,T,N,Q,D,E,K,R,L-Met sulfone-OH,L-hydroxy proline(tBu)-OH,(S)-2,4-diaminobutanoic acid or 2-(Boc)aminobutyric acid,L-ornithine(Boc)-OH,L-alpha-aminoadipic acid(delta tBu) or N-omega-(Pbf)-L-homoArg-OH
  MOD_RES 1: note = Beta-cyclopropyl-L-Ala-OH,Beta-cyclohexyl-L-Ala-OH, L-Met sulfone-OH,3-(4-prime-pyridyl)-L-Ala-OH,4-(Boc-amino )-L-Phe-OH,3-(4-thiazolyl)-L-Ala-OH,3,4-difluoro-L-Phe-OH, N-omega-(Pbf)-L-homoArg-OH or L-alpha-aminoadipic acid(delta tBu)
  MOD_RES 3..4: note = Beta-cyclopropyl-L-Ala-OH,Beta-cyclohexyl-L-Ala-OH, L-Met sulfone-OH,3-(4-prime-pyridyl)-L-Ala-OH,4-(Boc-amino )-L-Phe-OH,3-(4-thiazolyl)-L-Ala-OH,3,4-difluoro-L-Phe-OH, N-omega-(Pbf)-L-homoArg-OH or L-alpha-aminoadipic acid(delta tBu)
  MOD_RES 5: note = Beta-cyclopropyl-L-Ala-OH,Beta-cyclobutyl-L-Ala-OH, Beta-cyclohexyl-L-Ala-OH,L-2-aminooctanoic acid,4-fluoro-L -phenylalanine,3,4-difluoro-L-Phe-OH,3,4,5-trifluoro-L-Phe -OH,3-(4-thiazolyl)-L-Ala-OH,3-(2-naphtyl)-L-Ala-OH,HomoPh e-OH (hPh),3,4-dimethoxy-L-Phe-OH,4-phenylpiperidine-4-car boxylic acid or (4-aminomethyl) benzoic acid
  MOD_RES 7: note = Beta-cyclopropyl-L-Ala-OH,Beta-cyclohexyl-L-Ala-OH, L-Met sulfone-OH,3-(4-prime-pyridyl)-L-Ala-OH,4-(Boc-amino )-L-Phe-OH,3-(4-thiazolyl)-L-Ala-OH,3,4-difluoro-L-Phe-OH, N-omega-(Pbf)-L-homoArg-OH or L-alpha-aminoadipic acid(delta tBu)
  MOD_RES 8: note = L-Met sulfone-OH,L-hydroxyproline(tBu)-OH,(S)-2,4-d iaminobutanoic acid or 2-(Boc)aminobutyric acid,L-ornithine(Boc)-OH,L-alpha-aminoadipic acid(delta tBu) or N-omega-(Pbf)-L-homoArg-OH
XMXXXAXXLK

SEQ ID NO: 5 - protein, 9 aa, synthetic construct
  MOD_RES 1: note = 4-(Boc-amino)-L-Phe-OH
  MOD_RES 3: note = 3,4-difluoro-L-Phe-OH
  MOD_RES 7: note = Beta-cyclohexyl-L-Ala-OH
XMXSAAXRL

SEQ ID NO: 6 - protein, 9 aa, synthetic construct
  MOD_RES 1: note = N-omega-(Pbf)-L-homoArg-OH
  MOD_RES 3: note = L-Met sulfone-OH
  MOD_RES 4: note = 3,4-difluoro-L-Phe-OH
  MOD_RES 5: note = Beta-cyclohexyl-L-Ala-OH
  MOD_RES 8: note = L-Met sulfone-OH
XMXXXARXL

SEQ ID NO: 7 - protein, 9 aa, synthetic construct
  MOD_RES 3: note = N-omega-(Pbf)-L-homoArg-OH
  MOD_RES 4: note = 3,4-difluoro-L-Phe-OH
  MOD_RES 5: note = 3-(4-thiazolyl)-L-Ala-OH
  MOD_RES 7: note = Beta-cyclohexyl-L-Ala-OH
AMXXXAXRL

SEQ ID NO: 8 - protein, 9 aa, synthetic construct
  MOD_RES 1: note = 4-(Boc-amino)-L-Phe-OH
  MOD_RES 7: note = Beta-cyclohexyl-L-Ala-OH
XMASAAXRL

SEQ ID NO: 9 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XMXQXAXXL

SEQ ID NO: 10 - protein, 9 aa, synthetic construct
  MOD_RES 1: note = 4-(Boc-amino)-L-Phe-OH
  MOD_RES 3: note = 3,4-difluoro-L-Phe-OH
  MOD_RES 7: note = Beta-cyclohexyl-L-Ala-OH
XMXSAAXCL

SEQ ID NO: 11 - protein, 9 aa, synthetic construct
  MOD_RES 1: note = N-omega-(Pbf)-L-homoArg-OH
  MOD_RES 3: note = L-Met sulfone-OH
  MOD_RES 4: note = 3,4-difluoro-L-Phe-OH
  MOD_RES 5: note = Beta-cyclohexyl-L-Ala-OH
XMXXXARCL

SEQ ID NO: 12 - protein, 9 aa, synthetic construct
  MOD_RES 3: note = N-omega-(Pbf)-L-homoArg-OH
  MOD_RES 4: note = 3,4-difluoro-L-Phe-OH
  MOD_RES 5: note = 3-(4-thiazolyl)-L-Ala-OH
  MOD_RES 7: note = Beta-cyclohexyl-L-Ala-OH
AMXXXAXCL

SEQ ID NO: 13 - protein, 9 aa, synthetic construct
  MOD_RES 1: note = 4-(Boc-amino)-L-Phe-OH
  MOD_RES 7: note = Beta-cyclohexyl-L-Ala-OH
XMASAAXCL

SEQ ID NO: 14 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XMXQXAXCL

SEQ ID NO: 15 - protein, 9 aa, synthetic construct
  MOD_RES 1: note = 4-(Boc-amino)-L-Phe-OH
  MOD_RES 7: note = Beta-cyclohexyl-L-Ala-OH
XMCSAAXRL

SEQ ID NO: 16 - protein, 9 aa, synthetic construct
  MOD_RES 1: note = N-omega-(Pbf)-L-homoArg-OH
  MOD_RES 4: note = 3,4-difluoro-L-Phe-OH
  MOD_RES 5: note = Beta-cyclohexyl-L-Ala-OH
  MOD_RES 8: note = L-Met sulfone-OH
XMCXXARXL

SEQ ID NO: 17 - protein, 9 aa, synthetic construct
  MOD_RES 4: note = 3,4-difluoro-L-Phe-OH
  MOD_RES 5: note = 3-(4-thiazolyl)-L-Ala-OH
  MOD_RES 7: note = Beta-cyclohexyl-L-Ala-OH
AMCXXAXRL

SEQ ID NO: 18 - protein, 9 aa, synthetic construct
  MOD_RES 1: note = 4-(Boc-amino)-L-Phe-OH
  MOD_RES 7: note = Beta-cyclohexyl-L-Ala-OH
XMCSAAXRL

SEQ ID NO: 19 - protein, 9 aa, synthetic construct
  MOD_RES 1: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XMCQXAXXL

SEQ ID NO: 20-25 - skipped (6 entries)

SEQ ID NO: 26 - protein, 9 aa, synthetic construct
  MOD_RES 1: note = N-omega-(Pbf)-L-homoArg-OH
  MOD_RES 8: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXCQXAXXL

SEQ ID NO: 27 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 28 - protein, 9 aa, synthetic construct
  MOD_RES 1: note = N-omega-(Pbf)-L-homoArg-OH
  MOD_RES 2: note = O-methyl-L-homoserine
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XXCQXAXXL

SEQ ID NO: 29 - protein, 9 aa, synthetic construct
  MOD_RES 1: note = N-omega-(Pbf)-L-homoArg-OH
  MOD_RES 2: note = O-methyl-L-homoserine
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XXXQXAXCL

SEQ ID NO: 30 - protein, 9 aa, synthetic construct
  MOD_RES 1: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
  SITE 9: note = Nle
XMCQXAXXX

SEQ ID NO: 31 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  SITE 9: note = Nle
XMXQXAXCX

SEQ ID NO: 32-35 - skipped (4 entries)

SEQ ID NO: 36 - protein, 9 aa, synthetic construct
VMAPRTLFL

SEQ ID NO: 37 - protein, 9 aa, synthetic construct
CMAPRTLFL

SEQ ID NO: 38 - protein, 9 aa, synthetic construct
VCAPRTLFL

SEQ ID NO: 39 - protein, 9 aa, synthetic construct
VMCPRTLFL

SEQ ID NO: 40 - protein, 9 aa, synthetic construct
VMACRTLFL

SEQ ID NO: 41 - protein, 9 aa, synthetic construct
VMAPCTLFL

SEQ ID NO: 42 - protein, 9 aa, synthetic construct
VMAPRCLFL

SEQ ID NO: 43 - protein, 9 aa, synthetic construct
VMAPRTCFL

SEQ ID NO: 44 - protein, 9 aa, synthetic construct
VMAPRTLCL

SEQ ID NO: 45 - protein, 9 aa, synthetic construct
VMAPRTLFC

SEQ ID NO: 46 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 47 - protein, 8 aa, synthetic construct
VMAPTLFL

SEQ ID NO: 48 - protein, 296 aa, synthetic construct
MGSHSLKYFHTSVSRPGRGEPRFISVGYVDDTQFVRFDNDADRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVEWAGDGTFQKWAAVVVPSGEEQRYTCHVQHEGLPEPVTLRWKPASQPTIPIGSQDIFEAQKIEWHE

SEQ ID NO: 49 - protein, 100 aa, synthetic construct [sequence omitted]

SEQ ID NO: 50 - protein, 331 aa, synthetic construct
GHHHHHHHHGGKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLIGSGGGGSGGGGSGGGGSGGGGSGGGGSGGGGSGGPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNXLQVNRLKSAQCGSSIIYHCKHKL

SEQ ID NO: 51 - protein, 551 aa, synthetic construct
GQDIFEAQKIEAHEAPEAAGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGGGGKNSFTKLSIEPAFTPGPNIELQKDSDCCSCQEKWVGYRCNCYFISSEQKTWNESRHLCASQKSSLLQLQNTDELDFMSSSQQFYWIGLSYSEEHTAWLWENGSALSQYLFPSFETFNTKNCIAYNPNGNALDESCEDKNRYICKQQLIGSGGGGSGGGGSGGGGSGGGGSGGGGSGGGGSGGPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNXLQVNRLKSAQCGSSIIYHCKHKL

SEQ ID NO: 52 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
KXXQXAXXL

SEQ ID NO: 53 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 8: note = N-omega-(Pbf)-L-homoArg-OH
  MOD_RES 7: note = Beta-cyclohexyl-L-Ala-OH
XXXQKAXXL

SEQ ID NO: 54 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  SITE 2: note = Nle
XXXQXAXKL

SEQ ID NO: 55 - protein, 15 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XXXQXAXXLLPETGG

SEQ ID NO: 56 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 8: note = N-omega-(Pbf)-L-homoArg-OH
  MOD_RES 7: note = Beta-cyclohexyl-L-Ala-OH
XXXQKAXXL

SEQ ID NO: 57 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 8: note = N-omega-(Pbf)-L-homoArg-OH
  MOD_RES 7: note = Beta-cyclohexyl-L-Ala-OH
XXXQKAXXL

SEQ ID NO: 58 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 8: note = N-omega-(Pbf)-L-homoArg-OH
  MOD_RES 7: note = Beta-cyclohexyl-L-Ala-OH
XXXQKAXXL

SEQ ID NO: 59 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XXXQXAXKL

SEQ ID NO: 60 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 7: note = Beta-cyclohexyl-L-Ala-OH
  MOD_RES 8: note = N-omega-(Pbf)-L-homoArg-OH
XXXQKAXXL

SEQ ID NO: 61 - protein, 9 aa, synthetic construct
  MOD_RES 2: note = Beta-cyclohexyl-L-Ala-OH
  MOD_RES 7: note = 4-(Boc-amino)-L-Phe-OH
AXLAMRXAS

SEQ ID NO: 62 - protein, 15 aa, synthetic construct
  SITE 4: note = D-Proline
QQFPGNPNGNALDES

SEQ ID NO: 63 - protein, 15 aa, synthetic construct
  SITE 4: note = D-Proline
QQFPPNPNGNALDES

SEQ ID NO: 64 - protein, 15 aa, synthetic construct
  MOD_RES 5: note = Aib
QQFAXNPNGNALDES

SEQ ID NO: 65 - protein, 13 aa, synthetic construct
  MOD_RES 1: note = 1-amino-3,6,9,12-tetraoxapentadecan-15-oic acid
XNALDEYCEDKNR

SEQ ID NO: 66 - protein, 14 aa, synthetic construct
  MOD_RES 13: note = 2-(2-(2-aminoethoxy)ethoxy)acetic acid
NALDEYCEDKNRXK

SEQ ID NO: 67 - protein, 12 aa, synthetic construct
  MOD_RES 7: note = Lys(1-amino-3,6,9,12-tetraoxapentadecan-15-oic acid)
NALDEYXEDKNR

SEQ ID NO: 68 - protein, 19 aa, synthetic construct
  SITE 2: note = Nle
  MOD_RES 5: note = (S)-2,3-diaminopropanoic acid
VXAPXSICHGIEYPCTLFL

SEQ ID NO: 69 - protein, 20 aa, synthetic construct
  SITE 2: note = Nle
  MOD_RES 5: note = (S)-2,3-diaminopropanoic acid
VXAPXSPCERWAISFCTLFL

SEQ ID NO: 70 - protein, 20 aa, synthetic construct
  SITE 2: note = Nle
  MOD_RES 5: note = (S)-2,3-diaminopropanoic acid
VXAPXSHCSDTQEAYCTLFL

SEQ ID NO: 71 - protein, 20 aa, synthetic construct
  SITE 2: note = Nle
  MOD_RES 5: note = (S)-2,3-diaminopropanoic acid
VXAPXSWCTKTNDQRCTLFL

SEQ ID NO: 72 - protein, 20 aa, synthetic construct
  MOD_RES 5: note = (S)-2,3-diaminopropanoic acid
  SITE 2: note = Nle
VXAPXSFCNISGGIHCTLFL

SEQ ID NO: 73 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
AXXQXAXXL

SEQ ID NO: 74 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XAXQXAXXL

SEQ ID NO: 75 - protein, 9 aa, synthetic construct
  MOD_RES 1: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XXAQXAXXL

SEQ ID NO: 76 - skipped

SEQ ID NO: 77 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 7: note = Beta-cyclohexyl-L-Ala-OH
  MOD_RES 8: note = N-omega-(Pbf)-L-homoArg-OH
XXXQAAXXL

SEQ ID NO: 78 - skipped

SEQ ID NO: 79 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 8: note = N-omega-(Pbf)-L-homoArg-OH
XXXQXAAXL

SEQ ID NO: 80 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXAL

SEQ ID NO: 81-91 - skipped (11 entries)

SEQ ID NO: 92 - protein, 9 aa, synthetic construct
AMCGPRCLW

SEQ ID NO: 93 - protein, 9 aa, synthetic construct
ALCWRVPCT

SEQ ID NO: 94 - protein, 9 aa, synthetic construct
AMCPPSLCF

SEQ ID NO: 95 - protein, 9 aa, synthetic construct
AMCPPWMCF

SEQ ID NO: 96 - protein, 9 aa, synthetic construct
ALCGRSCLV

SEQ ID NO: 97 - protein, 9 aa, synthetic construct
AMCGARCLF

SEQ ID NO: 98 - protein, 9 aa, synthetic construct
AMCGRLCLF

SEQ ID NO: 99 - protein, 9 aa, synthetic construct
ALCTRVPCT

SEQ ID NO: 100 - protein, 9 aa, synthetic construct
ALCGKACLY

SEQ ID NO: 101 - protein, 9 aa, synthetic construct
ALCGARCLF

SEQ ID NO: 102 - protein, 9 aa, synthetic construct
AMCWSVPCS

SEQ ID NO: 103 - protein, 9 aa, synthetic construct
ALCPRVPCS

SEQ ID NO: 104 - protein, 9 aa, synthetic construct
AMCRQVPCH

SEQ ID NO: 105 - protein, 9 aa, synthetic construct
ALCSRVPCS

SEQ ID NO: 106 - protein, 9 aa, synthetic construct
ALCGRACLW

SEQ ID NO: 107 - protein, 10 aa, synthetic construct
  MOD_RES 2: note = N-omega-(Pbf)-L-homoArg-OH
  SITE 3: note = Nle
  MOD_RES 4: note = N-omega-(Pbf)-L-homoArg-OH
  MOD_RES 6: note = 4-fluoro-L-phenylalanine
  MOD_RES 8: note = Beta-cyclohexyl-L-Ala-OH
  MOD_RES 9: note = N-omega-(Pbf)-L-homoArg-OH
GXXXQXAXXL

SEQ ID NO: 108 - protein, 12 aa, synthetic construct
  MOD_RES 4: note = N-omega-(Pbf)-L-homoArg-OH
  SITE 5: note = Nle
  MOD_RES 6: note = N-omega-(Pbf)-L-homoArg-OH
  MOD_RES 8: note = 4-fluoro-L-phenylalanine
  MOD_RES 10: note = Beta-cyclohexyl-L-Ala-OH
  MOD_RES 11: note = N-omega-(Pbf)-L-homoArg-OH
GGGXXXQXAXXL

SEQ ID NO: 109 - protein, 14 aa, synthetic construct
  MOD_RES 6: note = N-omega-(Pbf)-L-homoArg-OH
  MOD_RES 8: note = N-omega-(Pbf)-L-homoArg-OH
  MOD_RES 13: note = N-omega-(Pbf)-L-homoArg-OH
  SITE 7: note = Nle
  MOD_RES 10: note = 4-fluoro-L-phenylalanine
  MOD_RES 12: note = Beta-cyclohexyl-L-Ala-OH
GGGGGXXXQXAXXL

SEQ ID NO: 110 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 111 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 112 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXKL

SEQ ID NO: 113 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 114 - protein, 9 aa, synthetic construct
CMAPRTLFL

SEQ ID NO: 115 - protein, 9 aa, synthetic construct
VCAPRTLFL

SEQ ID NO: 116 - protein, 9 aa, synthetic construct
VMCPRTLFL

SEQ ID NO: 117 - protein, 9 aa, synthetic construct
VMACRTLFL

SEQ ID NO: 118 - protein, 9 aa, synthetic construct
VMAPCTLFL

SEQ ID NO: 119 - protein, 9 aa, synthetic construct
VMAPRCLFL

SEQ ID NO: 120 - protein, 9 aa, synthetic construct
VMAPRTCFL

SEQ ID NO: 121 - protein, 9 aa, synthetic construct
VMAPRTLFC

SEQ ID NO: 122 - protein, 9 aa, synthetic construct
VMCPRTLCL

SEQ ID NO: 123 - protein, 9 aa, synthetic construct
  MOD_RES 1: note = N-omega-(Pbf)-L-homoArg-OH
  MOD_RES 8: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXKQXAXXL

SEQ ID NO: 124 - protein, 9 aa, synthetic construct
  MOD_RES 1: note = N-omega-(Pbf)-L-homoArg-OH
  MOD_RES 8: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXKQXAXXL

SEQ ID NO: 125 - protein, 9 aa, synthetic construct
  MOD_RES 3: note = N-omega-(Pbf)-L-homoArg-OH
  MOD_RES 1: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXKL

SEQ ID NO: 126 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXKL

SEQ ID NO: 127 - protein, 9 aa, synthetic construct
  MOD_RES 1: note = Beta-cyclohexyl-L-Ala-OH
  MOD_RES 2: note = N-omega-(Pbf)-L-homoArg-OH
  MOD_RES 5: note = N-omega-(Pbf)-L-homoArg-OH
  MOD_RES 9: note = N-omega-(Pbf)-L-homoArg-OH
  SITE 7: note = Nle
XXALXQXCX

SEQ ID NO: 128 - protein, 9 aa, synthetic construct
  MOD_RES 8: note = 2-aminoacrylic acid
VMAPRTLXL

SEQ ID NO: 129 - protein, 9 aa, synthetic construct
VMAPRTLCL

SEQ ID NO: 130 - protein, 9 aa, synthetic construct
  SITE 2: note = Nle
  MOD_RES 3: note = N-omega-(Pbf)-L-homoArg-OH
  MOD_RES 8: note = N-omega-(Pbf)-L-homoArg-OH
  MOD_RES 5: note = 4-fluoro-L-phenylalanine
  MOD_RES 7: note = Beta-cyclohexyl-L-Ala-OH
CXXQXAXXL

SEQ ID NO: 131 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 8: note = N-omega-(Pbf)-L-homoArg-OH
  MOD_RES 5: note = 4-fluoro-L-phenylalanine
  MOD_RES 7: note = Beta-cyclohexyl-L-Ala-OH
XCXQXAXXL

SEQ ID NO: 132-133 - skipped (2 entries)

SEQ ID NO: 134 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 135 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 136 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXXQXAXCL

SEQ ID NO: 137 - protein, 9 aa, synthetic construct
  MOD_RES 1: note = N-omega-(Pbf)-L-homoArg-OH
  MOD_RES 3: note = N-omega-(Pbf)-L-homoArg-OH
  SITE 2: note = Nle
  MOD_RES 7: note = Beta-cyclohexyl-L-Ala-OH
  MOD_RES 5: note = Lys(2-(2-(2-aminoethoxy)ethoxy)acetic acid)
XXXQXAXCL

SEQ ID NO: 138 - protein, 9 aa, synthetic construct
  MOD_RES 1: note = Beta-cyclohexyl-L-Ala-OH
  MOD_RES 5: note = N-omega-(Pbf)-L-homoArg-OH
  MOD_RES 9: note = N-omega-(Pbf)-L-homoArg-OH
  SITE 7: note = Nle
  MOD_RES 8: note = 4-fluoro-L-phenylalanine
XCALXQXXX

SEQ ID NO: 139 - protein, 9 aa, synthetic construct
  MOD_RES 1: note = N-omega-(Pbf)-L-homoArg-OH
  MOD_RES 8: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
XXCQXAXXL

SEQ ID NO: 140 - protein, 9 aa, synthetic construct
  MOD_RES 5: note = Lys(2-(2-(2-aminoethoxy)ethoxy)acetic acid)
VMAPXTLCL

SEQ ID NO: 141 - protein, 9 aa, synthetic construct
  MOD_RES 5: note = Lys(2-(2-(2-aminoethoxy)ethoxy)acetic acid)
VMAPXTLCL

SEQ ID NO: 142 - protein, 9 aa, synthetic construct
VMCPKTLFL

SEQ ID NO: 143 - protein, 9 aa, synthetic construct
  MOD_RES 5: note = Lys(1-amino-3,6,9,12-tetraoxapentadecan-15-oic acid)
VMCPXTLFL

SEQ ID NO: 144 - protein, 9 aa, synthetic construct
  MOD_RES 5: note = Lys(2-(2-(2-aminoethoxy)ethoxy)acetic acid)
VMCPXTLFL

SEQ ID NO: 145 - protein, 9 aa, synthetic construct
  MOD_RES 5: note = Lys(2-(2-(2-aminoethoxy)ethoxy)acetic acid)
VMAPXTLCL

SEQ ID NO: 146 - protein, 9 aa, synthetic construct
  MOD_RES 5: note = Lys(1-amino-3,6,9,12-tetraoxapentadecan-15-oic acid)
VMAPXTLCL

SEQ ID NO: 147 - protein, 9 aa, synthetic construct
VMAPKTLCL

SEQ ID NO: 148 - protein, 9 aa, synthetic construct
VMKPRTLCL

SEQ ID NO: 149 - protein, 9 aa, synthetic construct
VMAPRTLCL

SEQ ID NO: 150 - protein, 9 aa, synthetic construct
VMAPRTLCL

SEQ ID NO: 151 - protein, 9 aa, synthetic construct
VMAPRTLCL

SEQ ID NO: 152 - protein, 9 aa, synthetic construct
VMAPRTLCL

SEQ ID NO: 153 - protein, 9 aa, synthetic construct
VMAPRTLCL

SEQ ID NO: 154 - protein, 9 aa, synthetic construct
VMAPRTLCL

SEQ ID NO: 155 - protein, 9 aa, synthetic construct
  MOD_RES 1: note = 2,3-dimethylbutanoic acid
XMAPRTLCL

SEQ ID NO: 156 - protein, 9 aa, synthetic construct
  MOD_RES 1: note = (S)-2-hydroxy-4-methylpentanoic acid
XMAPRTLCL

SEQ ID NO: 157 - protein, 9 aa, synthetic construct
VMAPRTLCL

SEQ ID NO: 158 - protein, 9 aa, synthetic construct
  MOD_RES 8: note = (S)-2,4-diaminobutanoic acid or 2-(Boc)aminobutyric acid
VMAPRTLXL

SEQ ID NO: 159 - protein, 9 aa, synthetic construct
  MOD_RES 8: note = (S)-2,4-diaminobutanoic acid or 2-(Boc)aminobutyric acid
VMAPRTLXL

SEQ ID NO: 160 - protein, 9 aa, synthetic construct
  MOD_RES 8: note = (S)-2,4-diaminobutanoic acid or 2-(Boc)aminobutyric acid
VMAPRTLXL

SEQ ID NO: 161 - protein, 9 aa, synthetic construct
  MOD_RES 8: note = (S)-2,4-diaminobutanoic acid or 2-(Boc)aminobutyric acid
VMAPRTLXL

SEQ ID NO: 162 - protein, 9 aa, synthetic construct
  MOD_RES 8: note = (S)-2,3-diaminopropanoic acid
VMAPRTLXL

SEQ ID NO: 163 - protein, 9 aa, synthetic construct
  MOD_RES 8: note = (S)-2,3-diaminopropanoic acid
VMAPRTLXL

SEQ ID NO: 164 - protein, 9 aa, synthetic construct
  MOD_RES 8: note = (S)-2,3-diaminopropanoic acid
VMAPRTLXL

SEQ ID NO: 165 - protein, 9 aa, synthetic construct
  MOD_RES 8: note = (S)-2,3-diaminopropanoic acid
VMAPRTLXL

SEQ ID NO: 166 - protein, 9 aa, synthetic construct
  MOD_RES 1: note = (S)-2-hydroxy-3-methylbutanoic acid
XMAPRTLCL

SEQ ID NO: 167 - protein, 9 aa, synthetic construct
VMAPRTLCL

SEQ ID NO: 168 - protein, 9 aa, synthetic construct
VMAPRTLCL

SEQ ID NO: 169 - protein, 9 aa, synthetic construct
VMAPRTLCL

SEQ ID NO: 170 - protein, 9 aa, synthetic construct
VMAPRTLCL

SEQ ID NO: 171 - protein, 9 aa, synthetic construct
  SITE 2: note = Nle
VXAPRTLCL

SEQ ID NO: 172 - protein, 9 aa, synthetic construct
  SITE 2: note = Nle
  MOD_RES 8: note = Homo-L-cysteine
VXAPRTLXL

SEQ ID NO: 173 - protein, 9 aa, synthetic construct
  SITE 2: note = Nle
  MOD_RES 5: note = (S)-2-aminopent-4-ynoic acid
VXAPXTLCL

SEQ ID NO: 174 - protein, 9 aa, synthetic construct
  SITE 2: note = Nle
  MOD_RES 5: note = (S)-2-aminohept-6-ynoic acid
VXAPXTLCL

SEQ ID NO: 175 - protein, 9 aa, synthetic construct
VMAPATLCL

SEQ ID NO: 176 - protein, 9 aa, synthetic construct
VMAPKTLCL

SEQ ID NO: 177 - protein, 9 aa, synthetic construct
  SITE 5: note = D-Arginine
VMAPRTLCL

SEQ ID NO: 178 - protein, 9 aa, synthetic construct
  SITE 5: note = D-Arginine
VMAPRTLCL

SEQ ID NO: 179 - protein, 9 aa, synthetic construct
VMAPRTLCL

SEQ ID NO: 180 - protein, 9 aa, synthetic construct
VMAPRTLCL

SEQ ID NO: 181 - protein, 9 aa, synthetic construct
  MOD_RES 5: note = homoarginine
VMAPXTLCA

SEQ ID NO: 182 - protein, 9 aa, synthetic construct
  MOD_RES 5: note = L-canavanine
VMAPXTLCA

SEQ ID NO: 183 - protein, 9 aa, synthetic construct
  SITE 5: note = D-Arginine
VMAPRTLCA

SEQ ID NO: 184 - protein, 9 aa, synthetic construct
VMAPRTLCA

SEQ ID NO: 185 - protein, 9 aa, synthetic construct
VMAPRTACL

SEQ ID NO: 186 - protein, 9 aa, synthetic construct
VMAPRALCL

SEQ ID NO: 187 - protein, 9 aa, synthetic construct
VMAPATLCL

SEQ ID NO: 188 - protein, 9 aa, synthetic construct
VMAARTLCL

SEQ ID NO: 189 - protein, 9 aa, synthetic construct
VAAPRTLCL

SEQ ID NO: 190 - protein, 9 aa, synthetic construct
AMAPRTLCL

SEQ ID NO: 191 - protein, 9 aa, synthetic construct
VMGPRTLCL

SEQ ID NO: 192 - protein, 9 aa, synthetic construct
  MOD_RES 1: note = Homo-D-arginine
  SITE 1: note = Homo-D-arginine
  SITE 2: note = Nle
  MOD_RES 5: note = 4-fluoro-L-phenylalanine
  MOD_RES 7: note = Beta-cyclohexyl-L-Ala-OH
  MOD_RES 3: note = N6-carbamimidoyl-L-lysine
XXXQXAXCL

SEQ ID NO: 193 - protein, 9 aa, synthetic construct
  MOD_RES 1: note = Homo-D-arginine
  SITE 1: note = Homo-D-arginine
  SITE 2: note = Nle
  MOD_RES 5: note = 4-fluoro-L-phenylalanine
  MOD_RES 7: note = Beta-cyclohexyl-L-Ala-OH
  MOD_RES 3: note = N6-carbamimidoyl-L-lysine
XXXQXAXCL

SEQ ID NO: 194 - protein, 9 aa, synthetic construct
VMAPKTLFL

SEQ ID NO: 195 - protein, 12 aa, synthetic construct
AMCGPRCLWGGK

SEQ ID NO: 196 - protein, 12 aa, synthetic construct
AMCPPSLCFGGK

SEQ ID NO: 197 - protein, 12 aa, synthetic construct
ALCGRSCLVGGK

SEQ ID NO: 198 - protein, 12 aa, synthetic construct
AMCGRLCLFGGK

SEQ ID NO: 199 - protein, 12 aa, synthetic construct
ALCTRVPCTGGK

SEQ ID NO: 200 - protein, 12 aa, synthetic construct
ALCGKACLYGGK

SEQ ID NO: 201 - protein, 12 aa, synthetic construct
ALCGARCLFGGK

SEQ ID NO: 202 - protein, 12 aa, synthetic construct
AMCWSVPCSGGK

SEQ ID NO: 203 - protein, 12 aa, synthetic construct
ALCPRVPCSGGK

SEQ ID NO: 204 - protein, 12 aa, synthetic construct
AMCRQVPCHGGK

SEQ ID NO: 205 - protein, 12 aa, synthetic construct
ALCSRVPCSGGK

SEQ ID NO: 206 - protein, 12 aa, synthetic construct
ALCGRACLWGGK

SEQ ID NO: 207 - protein, 13 aa, synthetic construct
AMCGPRCLWLGGK

SEQ ID NO: 208 - protein, 13 aa, synthetic construct
AMCGPRCLFLGGK

SEQ ID NO: 209 - protein, 11 aa, synthetic construct
AMCGPRCLGGK

SEQ ID NO: 210 - protein, 12 aa, synthetic construct
AMCNRVPCTGGK

SEQ ID NO: 211 - protein, 12 aa, synthetic construct
AMCPTIPCSGGK

SEQ ID NO: 212 - protein, 12 aa, synthetic construct
ALCWRVRCTGGK

SEQ ID NO: 213 - protein, 12 aa, synthetic construct
ALCWRVPCTGGK

SEQ ID NO: 214 - protein, 12 aa, synthetic construct
ALCAPRLCLGGK

SEQ ID NO: 215 - protein, 12 aa, synthetic construct
ALCDPSKCFGGK

SEQ ID NO: 216 - protein, 12 aa, synthetic construct
ALCWHVPCQGGK

SEQ ID NO: 217 - protein, 12 aa, synthetic construct
ALCDPRWCFGGK

SEQ ID NO: 218 - protein, 12 aa, synthetic construct
AMCGKNCLFGGK

SEQ ID NO: 219 - protein, 12 aa, synthetic construct
ALCGRPCLYGGK

SEQ ID NO: 220 - protein, 12 aa, synthetic construct
AMCGKYCLFGGK

SEQ ID NO: 221 - protein, 12 aa, synthetic construct
ALCRWRCLYGGK

SEQ ID NO: 222 - protein, 12 aa, synthetic construct
ALCGWRCLYGGK

SEQ ID NO: 223 - protein, 12 aa, synthetic construct
AMCVPRCLWGGK

SEQ ID NO: 224 - protein, 12 aa, synthetic construct
ALCGIACFPGGK

SEQ ID NO: 225 - protein, 12 aa, synthetic construct
AMCGALCLFGGK

SEQ ID NO: 226 - protein, 9 aa, synthetic construct
ALCWRVPCT

SEQ ID NO: 227 - protein, 9 aa, synthetic construct
ALCAPRLCL

SEQ ID NO: 228 - protein, 9 aa, synthetic construct
ALCDPRWCF

SEQ ID NO: 229 - protein, 9 aa, synthetic construct
ALCGWRCLY

SEQ ID NO: 230 - protein, 9 aa, synthetic construct
AMCGALCLF

SEQ ID NO: 231 - protein, 9 aa, synthetic construct
  SITE 2: note = Nle
  MOD_RES 5: note = 4-fluoro-L-phenylalanine
  MOD_RES 1: note = N-omega-(Pbf)-L-homoArg-OH
  MOD_RES 3: note = N-omega-(Pbf)-L-homoArg-OH
  MOD_RES 7: note = Beta-cyclohexyl-L-Ala-OH
XXXQXAXCL

SEQ ID NO: 232 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
KXXQXAXXL

SEQ ID NO: 233 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 7: note = Beta-cyclohexyl-L-Ala-OH
  MOD_RES 8: note = N-omega-(Pbf)-L-homoArg-OH
XXXQKAXXL

SEQ ID NO: 234 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XXXQXAXKL

SEQ ID NO: 235 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
CXXQXAXXL

SEQ ID NO: 236 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XCXQXAXXL

SEQ ID NO: 237-238 - skipped (2 entries)

SEQ ID NO: 239 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 7: note = Beta-cyclohexyl-L-Ala-OH
XXXQKAXKL

SEQ ID NO: 240 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
CXXQXAXXL

SEQ ID NO: 241 - protein, 9 aa, synthetic construct
  MOD_RES 1: note = N-omega-(Pbf)-L-homoArg-OH
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XXXQXAXCL

SEQ ID NO: 242 - protein, 9 aa, synthetic construct
CMAPRTLFL

SEQ ID NO: 243 - protein, 9 aa, synthetic construct
VCAPRTLFL

SEQ ID NO: 244 - protein, 9 aa, synthetic construct
VMCPRTLFL

SEQ ID NO: 245 - protein, 9 aa, synthetic construct
VMACRTLFL

SEQ ID NO: 246 - protein, 9 aa, synthetic construct
VMAPCTLFL

SEQ ID NO: 247 - protein, 9 aa, synthetic construct
VMAPRCLFL

SEQ ID NO: 248 - protein, 9 aa, synthetic construct
VMAPRTCFL

SEQ ID NO: 249 - protein, 9 aa, synthetic construct
VMAPRTLCL

SEQ ID NO: 250 - protein, 9 aa, synthetic construct
VMAPRTLFC

SEQ ID NO: 251 - protein, 9 aa, synthetic construct
VMCPRTLCL

SEQ ID NO: 252-253 - skipped (2 entries)

SEQ ID NO: 254 - protein, 9 aa, synthetic construct
  MOD_RES 1: note = Beta-cyclohexyl-L-Ala-OH
  MOD_RES 2: note = N-omega-(Pbf)-L-homoArg-OH
  MOD_RES 5: note = N-omega-(Pbf)-L-homoArg-OH
  SITE 7: note = Nle
  MOD_RES 9: note = N-omega-(Pbf)-L-homoArg-OH
XXALXQXCX

SEQ ID NO: 255 - protein, 12 aa, synthetic construct
NALDEYCEDKNR

SEQ ID NO: 256 - protein, 12 aa, synthetic construct
  MOD_RES 7: note = Lys (2-(2-(2-aminoethoxy)ethoxy)acetic acid)
NALDEYKEDKNR

SEQ ID NO: 257 - protein, 9 aa, synthetic construct
  MOD_RES 1: note = Beta-cyclohexyl-L-Ala-OH
  MOD_RES 5: note = N-omega-(Pbf)-L-homoArg-OH
  SITE 7: note = Nle
  MOD_RES 8: note = 4-fluoro-L-phenylalanine
  MOD_RES 9: note = N-omega-(Pbf)-L-homoArg-OH
XCALXQXXX

SEQ ID NO: 258 - protein, 9 aa, synthetic construct
  MOD_RES 1: note = N-omega-(Pbf)-L-homoArg-OH
  SITE 2: note = Nle
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XXCQXAXXL

SEQ ID NO: 259 - protein, 9 aa, synthetic construct
  MOD_RES 5: note = Lys (2-(2-(2-aminoethoxy)ethoxy)acetic acid)
VMAPKTLCL

SEQ ID NO: 260 - protein, 9 aa, synthetic construct
  MOD_RES 5: note = Lys (2-(2-(2-aminoethoxy)ethoxy)acetic acid)
VMAPKTLCL

SEQ ID NO: 261 - protein, 9 aa, synthetic construct
VMCPKTLFL

SEQ ID NO: 262 - protein, 9 aa, synthetic construct
VMCPKTLFL

SEQ ID NO: 263 - protein, 9 aa, synthetic construct
  MOD_RES 5: note = Lys (1-amino-3,6,9,12-tetraoxapentadecan-15-oic acid)
VMCPKTLFL

SEQ ID NO: 264 - protein, 9 aa, synthetic construct
  MOD_RES 5: note = Lys (2-(2-(2-aminoethoxy)ethoxy)acetic acid)
VMCPKTLFL

SEQ ID NO: 265 - protein, 9 aa, synthetic construct
  MOD_RES 5: note = Lys (2-(2-(2-aminoethoxy)ethoxy)acetic acid)
VMAPKTLCL

SEQ ID NO: 266 - protein, 9 aa, synthetic construct
  MOD_RES 5: note = Lys (1-amino-3,6,9,12-tetraoxapentadecan-15-oic acid)
VMAPKTLCL

SEQ ID NO: 267 - skipped

SEQ ID NO: 268 - protein, 9 aa, synthetic construct
VMCPKTLFL

SEQ ID NO: 269 - protein, 13 aa, synthetic construct
  MOD_RES 6: note = 2,5,8,11,14-pentaoxaheptadecan-17-oic acid
VMAPKXKKKTLCL

SEQ ID NO: 270 - protein, 6 aa, synthetic construct
  MOD_RES 1: note = (S)-2-aminohept-6-ynoic acid
XVNRLK

SEQ ID NO: 271 - protein, 5 aa, synthetic construct
  MOD_RES 1: note = (S)-2-aminohept-6-ynoic acid
XSQQF

SEQ ID NO: 272 - protein, 9 aa, synthetic construct
VMAPKTLCL

SEQ ID NO: 273 - protein, 9 aa, synthetic construct
VMKPRTLCL

SEQ ID NO: 274 - protein, 9 aa, synthetic construct
  MOD_RES 5: note = (S)-2-aminohept-6-ynoic acid
VMAPXTLCL

SEQ ID NO: 275-277 - skipped (3 entries)

SEQ ID NO: 278 - protein, 11 aa, synthetic construct
SFCNISGGIHC

SEQ ID NO: 279 - protein, 11 aa, synthetic construct
SWCTKTNDQRC

SEQ ID NO: 280 - protein, 11 aa, synthetic construct
SHCSDTQEAYC

SEQ ID NO: 281 - protein, 11 aa, synthetic construct
SPCERWAISFC

SEQ ID NO: 282 - protein, 10 aa, synthetic construct
SICHGIEYPC

SEQ ID NO: 283 - protein, 9 aa, synthetic construct
  SITE 2: note = Nle
  MOD_RES 5: note = (S)-2,3-diaminopropanoic acid
VXAPXTLFL

SEQ ID NO: 284 - protein, 11 aa, synthetic construct
SFCNISGGIHC

SEQ ID NO: 285 - protein, 11 aa, synthetic construct
SWCTKTNDQRC

SEQ ID NO: 286 - protein, 11 aa, synthetic construct
SHCSDTQEAYC

SEQ ID NO: 287 - protein, 11 aa, synthetic construct
SPCERWAISFC

SEQ ID NO: 288 - protein, 10 aa, synthetic construct
SICHGIEYPC

SEQ ID NO: 289 - protein, 9 aa, synthetic construct
LCTLVPMAR

SEQ ID NO: 290 - protein, 9 aa, synthetic construct
LFTLVPMAR

SEQ ID NO: 291 - protein, 9 aa, synthetic construct
LCTLVPMAR

SEQ ID NO: 292 - protein, 9 aa, synthetic construct
  MOD_RES 1: note = 2,3-dimethylbutanoic acid
XMAPRTLCL

SEQ ID NO: 293 - protein, 9 aa, synthetic construct
  MOD_RES 1: note = (S)-2-hydroxy-4-methylpentanoic acid
XMAPRTLCL

SEQ ID NO: 294 - protein, 9 aa, synthetic construct
VMAPRTLCL

SEQ ID NO: 295 - protein, 9 aa, synthetic construct
  MOD_RES 8: note = (S)-2,4-diaminobutanoic acid or 2-(Boc)aminobutyric acid
VMAPRTLXL

SEQ ID NO: 296 - protein, 9 aa, synthetic construct
  MOD_RES 8: note = (S)-2,3-diaminopropanoic acid
VMAPRTLXL

SEQ ID NO: 297 - protein, 9 aa, synthetic construct
  MOD_RES 1: note = (S)-2-hydroxy-3-methylbutanoic acid
XMAPRTLCL

SEQ ID NO: 298 - protein, 9 aa, synthetic construct
VMAPCTLFL

SEQ ID NO: 299 - protein, 9 aa, synthetic construct
  SITE 2: note = Nle
VXAPRTLCL

SEQ ID NO: 300 - protein, 9 aa, synthetic construct
  SITE 2: note = Nle
  MOD_RES 8: note = Homo-L-cysteine
VXAPRTLXL

SEQ ID NO: 301 - protein, 9 aa, synthetic construct
  SITE 2: note = Nle
  MOD_RES 5: note = (S)-2-aminopent-4-ynoic acid
VXAPXTLCL

SEQ ID NO: 302 - protein, 9 aa, synthetic construct
  SITE 2: note = Nle
  MOD_RES 5: note = (S)-2-aminohept-6-ynoic acid
VXAPXTLCL

SEQ ID NO: 303 - protein, 9 aa, synthetic construct
PRLMALVCT

SEQ ID NO: 304 - protein, 9 aa, synthetic construct
PRLMALVCT

SEQ ID NO: 305 - protein, 9 aa, synthetic construct
PRLMCLVFT

SEQ ID NO: 306 - protein, 9 aa, synthetic construct
PRLMCLVFT

SEQ ID NO: 307 - protein, 9 aa, synthetic construct
VMAPATLCL

SEQ ID NO: 308 - protein, 9 aa, synthetic construct
VMAPKTLCL

SEQ ID NO: 309 - protein, 9 aa, synthetic construct
  MOD_RES 5: note = homoarginine
VMAPXTLCL

SEQ ID NO: 310 - protein, 9 aa, synthetic construct
  MOD_RES 5: note = L-canavanine
VMAPXTLCL

SEQ ID NO: 311 - protein, 9 aa, synthetic construct
  SITE 5: note = D-Arginine
VMAPRTLCL

SEQ ID NO: 312 - protein, 9 aa, synthetic construct
VMAPRTLCA

SEQ ID NO: 313 - protein, 9 aa, synthetic construct
VMAPRTACL

SEQ ID NO: 314 - protein, 9 aa, synthetic construct
VMAPRALCL

SEQ ID NO: 315 - protein, 9 aa, synthetic construct
VMAPATLCL

SEQ ID NO: 316 - protein, 9 aa, synthetic construct
VMAARTLCL

SEQ ID NO: 317 - protein, 9 aa, synthetic construct
VMGPRTLCL

SEQ ID NO: 318 - protein, 9 aa, synthetic construct
VAAPRTLCL

SEQ ID NO: 319 - protein, 9 aa, synthetic construct
AMAPRTLCL

SEQ ID NO: 320 - protein, 12 aa, synthetic construct
ALCSRVPCSGGK

SEQ ID NO: 321 - protein, 12 aa, synthetic construct
ALCEPSLCLGGK

SEQ ID NO: 322 - protein, 12 aa, synthetic construct
ALCGRSCLVGGK

SEQ ID NO: 323 - protein, 12 aa, synthetic construct
ALCGLSCLVGGK

SEQ ID NO: 324 - protein, 12 aa, synthetic construct
ALCGASCLFGGK

SEQ ID NO: 325 - protein, 12 aa, synthetic construct
ALCGSRCLFGGK

SEQ ID NO: 326 - protein, 12 aa, synthetic construct
ALCGSACLYGGK

SEQ ID NO: 327 - protein, 12 aa, synthetic construct
ALCGRHCLFGGK

SEQ ID NO: 328 - protein, 12 aa, synthetic construct
ALCGRTCLTGGK

SEQ ID NO: 329 - protein, 12 aa, synthetic construct
ALCGRACLWGGK

SEQ ID NO: 330 - protein, 12 aa, synthetic construct
AMCGRLCLFGGK

SEQ ID NO: 331 - protein, 12 aa, synthetic construct
AMCGLPCLVGGK

SEQ ID NO: 332 - skipped

SEQ ID NO: 333 - protein, 9 aa, synthetic construct
  MOD_RES 1: note = Homo-D-arginine
  SITE 1: note = D-Arginine
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
XXXQXAXCL

SEQ ID NO: 334 - protein, 12 aa, synthetic construct
ALCWRVPCTGGK

SEQ ID NO: 335 - protein, 12 aa, synthetic construct
AMCPPWMCFGGK

SEQ ID NO: 336 - protein, 12 aa, synthetic construct
AMCGARCLFGGK

SEQ ID NO: 337 - protein, 12 aa, synthetic construct
  SITE 2: note = Nle
AXCGPRCLWGGK

SEQ ID NO: 338 - protein, 11 aa, synthetic construct
MCGPRCLWGGK

SEQ ID NO: 339 - protein, 12 aa, synthetic construct
ALCWRVSCTGGK

SEQ ID NO: 340 - protein, 12 aa, synthetic construct
GMCGPRCLWGGK

SEQ ID NO: 341 - protein, 12 aa, synthetic construct
AACGPRCLWGGK

SEQ ID NO: 342 - protein, 12 aa, synthetic construct
AMCAPRCLWGGK

SEQ ID NO: 343 - protein, 12 aa, synthetic construct
AMCGARCLWGGK

SEQ ID NO: 344 - protein, 12 aa, synthetic construct
AMCGPACLWGGK

SEQ ID NO: 345 - protein, 12 aa, synthetic construct
AMCGPRCAWGGK

SEQ ID NO: 346 - protein, 12 aa, synthetic construct
AMCGPRCLAGGK

SEQ ID NO: 347 - protein, 17 aa, synthetic construct
  SITE 4: note = D-Proline
  MOD_RES 16: note = 3-aminopropanoic acid
QQFPGNPNGNALDESXK

SEQ ID NO: 348 - protein, 17 aa, synthetic construct
  SITE 4: note = D-Proline
  MOD_RES 16: note = 3-aminopropanoic acid
QQFPPNPNGNALDESXK

SEQ ID NO: 349 - protein, 17 aa, synthetic construct
  MOD_RES 5: note = Aib
  MOD_RES 16: note = 3-aminopropanoic acid
QQFAXNPNGNALDESXK

SEQ ID NO: 350 - protein, 9 aa, synthetic construct
  SITE 2: note = Nle
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
CXXQXAXXL

SEQ ID NO: 351 - protein, 9 aa, synthetic construct
  SITE 2: note = Nle
  MOD_RES 3: note = N-omega-(Pbf)-L-homoArg-OH
  MOD_RES 8: note = N-omega-(Pbf)-L-homoArg-OH
  MOD_RES 5: note = 4-fluoro-L-phenylalanine
  MOD_RES 7: note = Beta-cyclohexyl-L-Ala-OH
CXXQXAXXL

SEQ ID NO: 352 - skipped

SEQ ID NO: 353 - protein, 9 aa, synthetic construct
VMAPRTLCL

SEQ ID NO: 354 - protein, 12 aa, synthetic construct
VMAPATLFLGGK

SEQ ID NO: 355 - protein, 9 aa, synthetic construct
AMAPRTLLL

SEQ ID NO: 356 - protein, 39 aa, synthetic construct
GSGGGGSGGGGSGGGGSGGGGSGGGGSGGGGSGGGGSGG

SEQ ID NO: 357 - protein, 8 aa, synthetic construct
HHHHHHHH

SEQ ID NO: 358 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 359 - protein, 9 aa, synthetic construct
  MOD_RES 1: note = Beta-cyclohexyl-L-Ala-OH
  MOD_RES 2: note = N-omega-(Pbf)-L-homoArg-OH
  MOD_RES 5: note = N-omega-(Pbf)-L-homoArg-OH
  MOD_RES 9: note = N-omega-(Pbf)-L-homoArg-OH
  MOD_RES 8: note = 4-fluoro-L-phenylalanine
XXALXQMXX

SEQ ID NO: 360 - skipped

SEQ ID NO: 361 - protein, 8 aa, synthetic construct
MAPRTLFL

SEQ ID NO: 362 - protein, 9 aa, synthetic construct
  MOD_RES 3: note = N-omega-(Pbf)-L-homoArg-OH
  MOD_RES 4: note = 3,4-difluoro-L-Phe-OH
  MOD_RES 5: note = 3-(4-thiazolyl)-L-Ala-OH
  MOD_RES 7: note = Beta-cyclohexyl-L-Ala-OH
AMXXXAXRL

SEQ ID NO: 363 - protein, 9 aa, synthetic construct
  MOD_RES 1: note = Beta-cyclopropyl-L-Ala-OH
  MOD_RES 4: note = 3,4-difluoro-L-Phe-OH
  MOD_RES 7: note = 4-(Boc-amino)-L-Phe-OH
  MOD_RES 8: note = N-omega-(Pbf)-L-homoArg-OH
XMSXWAXXL

SEQ ID NO: 364 - protein, 9 aa, synthetic construct
  MOD_RES 2: note = Met(O)
  MOD_RES 4: note = Beta-cyclopropyl-L-Ala-OH
  MOD_RES 7: note = Beta-cyclohexyl-L-Ala-OH
AXDXMAXRL

SEQ ID NO: 365 - protein, 9 aa, synthetic construct
  MOD_RES 2: note = Met(O)
  MOD_RES 3: note = L-alpha-aminoadipic acid(delta tBu)
  MOD_RES 5: note = L-2-aminooctanoic acid
  MOD_RES 7: note = Met(O)
AXXSXAXSL

SEQ ID NO: 366 - protein, 9 aa, synthetic construct
  MOD_RES 1: note = Beta-cyclopropyl-L-Ala-OH
  MOD_RES 2: note = Met(O)
  MOD_RES 4: note = 3,4-difluoro-L-Phe-OH
  MOD_RES 7: note = 4-(Boc-amino)-L-Phe-OH
  MOD_RES 8: note = N-omega-(Pbf)-L-homoArg-OH
XXSXWAXXL

SEQ ID NO: 367 - protein, 9 aa, synthetic construct
  MOD_RES 1: note = Beta-cyclohexyl-L-Ala-OH
  MOD_RES 4: note = Beta-cyclohexyl-L-Ala-OH
  MOD_RES 7: note = 3-(4-thiazolyl)-L-Ala-OH
XMDXLAXDL

SEQ ID NO: 368 - protein, 9 aa, synthetic construct
  MOD_RES 1: note = Beta-cyclohexyl-L-Ala-OH
  MOD_RES 3: note = L-Met sulfone-OH
  MOD_RES 4: note = N-omega-(Pbf)-L-homoArg-OH
  MOD_RES 7: note = 3,4-difluoro-L-Phe-OH
  MOD_RES 8: note = L-alpha-aminoadipic acid(delta tBu)
XMXXLAXXL

SEQ ID NO: 369 - protein, 9 aa, synthetic construct
  MOD_RES 1: note = Beta-cyclohexyl-L-Ala-OH
  MOD_RES 2: note = Met(O)
  MOD_RES 4: note = Beta-cyclohexyl-L-Ala-OH
  MOD_RES 7: note = 3-(4-thiazolyl)-L-Ala-OH
XXDXLAXDL

SEQ ID NO: 370 - protein, 9 aa, synthetic construct
  MOD_RES 4: note = Beta-cyclohexyl-L-Ala-OH
  MOD_RES 5: note = 3,4,5-trifluoro-L-Phe-OH
  MOD_RES 7: note = L-alpha-aminoadipic acid(delta tBu)
DMAXXAXQL

SEQ ID NO: 371 - protein, 9 aa, synthetic construct
  MOD_RES 4: note = 3-(4-thiazolyl)-L-Ala-OH
  MOD_RES 7: note = L-alpha-aminoadipic acid(delta tBu)
  MOD_RES 8: note = L-Met sulfone-OH
DMWXVAXXL

SEQ ID NO: 372 - protein, 9 aa, synthetic construct
  MOD_RES 2: note = Met(O)
  MOD_RES 4: note = Beta-cyclohexyl-L-Ala-OH
  MOD_RES 5: note = 3,4,5-trifluoro-L-Phe-OH
  MOD_RES 7: note = L-alpha-aminoadipic acid(delta tBu)
DXAXXAXQL

SEQ ID NO: 373 - protein, 9 aa, synthetic construct
  MOD_RES 1: note = 4-(Boc-amino)-L-Phe-OH
  MOD_RES 4: note = Beta-cyclopropyl-L-Ala-OH
  MOD_RES 5: note = HomoPhe-OH (hPh)
  MOD_RES 7: note = 3-(4-prime-pyridyl)-L-Ala-OH
XMAXXAXEL

SEQ ID NO: 374 - protein, 9 aa, synthetic construct
  MOD_RES 1: note = 4-(Boc-amino)-L-Phe-OH
  MOD_RES 7: note = Beta-cyclohexyl-L-Ala-OH
XMASAAXRL

SEQ ID NO: 375 - protein, 9 aa, synthetic construct
  MOD_RES 1: note = 4-(Boc-amino)-L-Phe-OH
  MOD_RES 7: note = Beta-cyclohexyl-L-Ala-OH
XMFSAAXRL

SEQ ID NO: 376 - protein, 9 aa, synthetic construct
  MOD_RES 1: note = 4-(Boc-amino)-L-Phe-OH
  MOD_RES 3: note = 3,4-difluoro-L-Phe-OH
  MOD_RES 7: note = Beta-cyclohexyl-L-Ala-OH
XMXSAAXRL

SEQ ID NO: 377 - protein, 9 aa, synthetic construct
  MOD_RES 1: note = 4-(Boc-amino)-L-Phe-OH
  MOD_RES 2: note = Met(O)
  MOD_RES 7: note = Beta-cyclohexyl-L-Ala-OH
XXASAAXRL

SEQ ID NO: 378 - protein, 9 aa, synthetic construct
  MOD_RES 1: note = 4-(Boc-amino)-L-Phe-OH
  MOD_RES 2: note = Met(O)
  MOD_RES 7: note = Beta-cyclohexyl-L-Ala-OH
XXFSAAXRL

SEQ ID NO: 379 - protein, 9 aa, synthetic construct
  MOD_RES 1: note = 4-(Boc-amino)-L-Phe-OH
  MOD_RES 2: note = Met(O)
  MOD_RES 3: note = 3,4-difluoro-L-Phe-OH
  MOD_RES 7: note = Beta-cyclohexyl-L-Ala-OH
XXXDAAXRL

SEQ ID NO: 380 - protein, 9 aa, synthetic construct
  MOD_RES 1: note = 4-(Boc-amino)-L-Phe-OH
  MOD_RES 2: note = Met(O)
  MOD_RES 3: note = 3,4-difluoro-L-Phe-OH
  MOD_RES 7: note = Beta-cyclohexyl-L-Ala-OH
XXXSAAXRL

SEQ ID NO: 381 - protein, 9 aa, synthetic construct
  MOD_RES 7: note = L-alpha-aminoadipic acid(delta tBu)
FMAYAAXRL

SEQ ID NO: 382 - protein, 9 aa, synthetic construct
  MOD_RES 1: note = 3-(4-thiazolyl)-L-Ala-OH
  MOD_RES 4: note = 3-(4-prime-pyridyl)-L-Ala-OH
  MOD_RES 5: note = 3-(2-naphtyl)-L-Ala-OH
XMYXXASRL

SEQ ID NO: 383 - skipped

SEQ ID NO: 384 - protein, 9 aa, synthetic construct
  MOD_RES 1: note = 3,4-difluoro-L-Phe-OH
  MOD_RES 7: note = 3-(4-prime-pyridyl)-L-Ala-OH
XMAYFAXEL

SEQ ID NO: 385 - protein, 9 aa, synthetic construct
  MOD_RES 1: note = 3,4-difluoro-L-Phe-OH
  MOD_RES 7: note = 4-(Boc-amino)-L-Phe-OH
XMAYFAXDL

SEQ ID NO: 386 - protein, 9 aa, synthetic construct
  MOD_RES 1: note = 3,4-difluoro-L-Phe-OH
  MOD_RES 4: note = Beta-cyclohexyl-L-Ala-OH
  MOD_RES 5: note = L-2-aminooctanoic acid
  MOD_RES 7: note = L-alpha-aminoadipic acid(delta tBu)
XMEXXAXKL

SEQ ID NO: 387 - protein, 9 aa, synthetic construct
  MOD_RES 1: note = 3,4-difluoro-L-Phe-OH
  MOD_RES 5: note = 3,4,5-trifluoro-L-Phe-OH
  MOD_RES 7: note = L-alpha-aminoadipic acid(delta tBu)
XMYSXAXRL

SEQ ID NO: 388 - protein, 9 aa, synthetic construct
  MOD_RES 1: note = 3,4-difluoro-L-Phe-OH
  MOD_RES 2: note = Met(O)
  MOD_RES 3: note = Beta-cyclopropyl-L-Ala-OH
  MOD_RES 5: note = 3,4,5-trifluoro-L-Phe-OH
  MOD_RES 8: note = L-Met sulfone-OH
XXXSXAHXL

SEQ ID NO: 389 - protein, 9 aa, synthetic construct
  MOD_RES 1: note = 3,4-difluoro-L-Phe-OH
  MOD_RES 2: note = Met(O)
  MOD_RES 7: note = 3-(4-prime-pyridyl)-L-Ala-OH
XXAYFAXEL

SEQ ID NO: 390 - protein, 9 aa, synthetic construct
  MOD_RES 1: note = 3,4-difluoro-L-Phe-OH
  MOD_RES 2: note = Met(O)
  MOD_RES 7: note = 4-(Boc-amino)-L-Phe-OH
XXAYFAXDL

SEQ ID NO: 391 - protein, 9 aa, synthetic construct
  MOD_RES 1: note = 3,4-difluoro-L-Phe-OH
  MOD_RES 2: note = Met(O)
  MOD_RES 4: note = Beta-cyclohexyl-L-Ala-OH
  MOD_RES 5: note = L-2-aminooctanoic acid
  MOD_RES 7: note = L-alpha-aminoadipic acid(delta tBu)
XXEXXAXKL

SEQ ID NO: 392 - protein, 9 aa, synthetic construct
  MOD_RES 1: note = 3,4-difluoro-L-Phe-OH
  MOD_RES 2: note = Met(O)
  MOD_RES 3: note = L-alpha-aminoadipic acid(delta tBu)
  MOD_RES 4: note = Beta-cyclopropyl-L-Ala-OH
  MOD_RES 7: note = 3-(4-prime-pyridyl)-L-Ala-OH
XXXXFAXEL

SEQ ID NO: 393 - protein, 9 aa, synthetic construct
  MOD_RES 1: note = L-Met sulfone-OH
  MOD_RES 2: note = Met(O)
  MOD_RES 7: note = 3-(4-prime-pyridyl)-L-Ala-OH
XXAYFAXEL

SEQ ID NO: 394 - protein, 9 aa, synthetic construct
  MOD_RES 1: note = N-omega-(Pbf)-L-homoArg-OH
  MOD_RES 7: note = 3-(4-prime-pyridyl)-L-Ala-OH
XMAYFAXEL

SEQ ID NO: 395 - protein, 9 aa, synthetic construct
  MOD_RES 1: note = N-omega-(Pbf)-L-homoArg-OH
  MOD_RES 3: note = L-Met sulfone-OH
  MOD_RES 4: note = 3,4-difluoro-L-Phe-OH
  MOD_RES 5: note = Beta-cyclohexyl-L-Ala-OH
  MOD_RES 8: note = L-Met sulfone-OH
XMXXXARXL

SEQ ID NO: 396 - protein, 9 aa, synthetic construct
  MOD_RES 1: note = N-omega-(Pbf)-L-homoArg-OH
  MOD_RES 3: note = N-omega-(Pbf)-L-homoArg-OH
  MOD_RES 5: note = 4-fluoro-L-phenylalanine
  MOD_RES 7: note = Beta-cyclohexyl-L-Ala-OH
  MOD_RES 8: note = N-omega-(Pbf)-L-homoArg-OH
XMXQXAXXL

SEQ ID NO: 397 - protein, 9 aa, synthetic construct
  MOD_RES 1: note = N-omega-(Pbf)-L-homoArg-OH
  MOD_RES 2: note = Met(O)
  MOD_RES 7: note = 3-(4-prime-pyridyl)-L-Ala-OH
XXAYFAXEL

SEQ ID NO: 398 - protein, 9 aa, synthetic construct
  MOD_RES 1: note = N-omega-(Pbf)-L-homoArg-OH
  MOD_RES 2: note = Met(O)
  MOD_RES 7: note = L-Met sulfone-OH
  MOD_RES 8: note = L-ornithine(Boc)-OH
XXAYFAXXL

SEQ ID NO: 399 - skipped

SEQ ID NO: 400 - protein, 9 aa, synthetic construct
  MOD_RES 1: note = L-alpha-aminoadipic acid(delta tBu)
  MOD_RES 3: note = 3-(4-thiazolyl)-L-Ala-OH
  MOD_RES 4: note = Beta-cyclopropyl-L-Ala-OH
  MOD_RES 5: note = HomoPhe-OH (hPh)
  MOD_RES 7: note = 3-(4-prime-pyridyl)-L-Ala-OH
XMXXXAXEL

SEQ ID NO: 401-403 - skipped (3 entries)

SEQ ID NO: 404 - protein, 9 aa, synthetic construct
  MOD_RES 4: note = Beta-cyclohexyl-L-Ala-OH
  MOD_RES 7: note = 3,4-difluoro-L-Phe-OH
  MOD_RES 8: note = N-omega-(Pbf)-L-homoArg-OH
MMWXMAXXL

SEQ ID NO: 405 - protein, 9 aa, synthetic construct
  MOD_RES 4: note = 3-(4-prime-pyridyl)-L-Ala-OH
  MOD_RES 5: note = 3-(2-naphtyl)-L-Ala-OH
PMYXXARSL

SEQ ID NO: 406 - protein, 9 aa, synthetic construct
  MOD_RES 4: note = 3-(4-prime-pyridyl)-L-Ala-OH
  MOD_RES 5: note = 3-(2-naphtyl)-L-Ala-OH
PMYXXASRL

SEQ ID NO: 407 - protein, 9 aa, synthetic construct
  MOD_RES 2: note = Met(O)
  MOD_RES 4: note = Beta-cyclopropyl-L-Ala-OH
  MOD_RES 5: note = L-2-aminooctanoic acid
PXFXXASKL

SEQ ID NO: 408 - protein, 9 aa, synthetic construct
  MOD_RES 4: note = L-Met sulfone-OH
  MOD_RES 5: note = Beta-cyclopropyl-L-Ala-OH
  MOD_RES 8: note = N-omega-(Pbf)-L-homoArg-OH
QMWXXAMXL

SEQ ID NO: 409 - protein, 9 aa, synthetic construct
  MOD_RES 2: note = Met(O)
  MOD_RES 4: note = L-Met sulfone-OH
  MOD_RES 5: note = Beta-cyclopropyl-L-Ala-OH
  MOD_RES 7: note = Met(O)
  MOD_RES 8: note = N-omega-(Pbf)-L-homoArg-OH
QXWXXAXXL

SEQ ID NO: 410 - protein, 9 aa, synthetic construct
  MOD_RES 3: note = N-omega-(Pbf)-L-homoArg-OH
  MOD_RES 4: note = 3-(4-thiazolyl)-L-Ala-OH
  MOD_RES 8: note = N-omega-(Pbf)-L-homoArg-OH
RMXXFAPXL

SEQ ID NO: 411 - protein, 9 aa, synthetic construct
  MOD_RES 4: note = Beta-cyclopropyl-L-Ala-OH
  MOD_RES 7: note = Beta-cyclohexyl-L-Ala-OH
SMDXMAXRL

SEQ ID NO: 412 - protein, 9 aa, synthetic construct
  MOD_RES 4: note = Beta-cyclopropyl-L-Ala-OH
  MOD_RES 5: note = Met(O)
  MOD_RES 7: note = Beta-cyclohexyl-L-Ala-OH
SMDXXAXRL

SEQ ID NO: 413 - protein, 9 aa, synthetic construct
  MOD_RES 2: note = Met(O)
  MOD_RES 4: note = Beta-cyclopropyl-L-Ala-OH
  MOD_RES 7: note = Beta-cyclohexyl-L-Ala-OH
SXDXMAXRL

SEQ ID NO: 414 - protein, 9 aa, synthetic construct
  MOD_RES 2: note = Met(O)
  MOD_RES 4: note = Beta-cyclopropyl-L-Ala-OH
  MOD_RES 5: note = Met(O)
  MOD_RES 7: note = Beta-cyclohexyl-L-Ala-OH
SXDXVAXRL

SEQ ID NO: 415 - protein, 9 aa, synthetic construct
  MOD_RES 2: note = Met(O)
  MOD_RES 4: note = Beta-cyclohexyl-L-Ala-OH
  MOD_RES 7: note = 3,4-difluoro-L-Phe-OH
  MOD_RES 8: note = N-omega-(Pbf)-L-homoArg-OH
SXRXVAXXL

SEQ ID NO: 416 - protein, 9 aa, synthetic construct
  MOD_RES 2: note = Met(O)
  MOD_RES 4: note = 3,4-difluoro-L-Phe-OH
  MOD_RES 7: note = 3-(4-thiazolyl)-L-Ala-OH
  MOD_RES 8: note = L-ornithine(Boc)-OH
SXYXVAXXL

SEQ ID NO: 417 - protein, 9 aa, synthetic construct
  MOD_RES 7: note = 4-(Boc-amino)-L-Phe-OH
YMAYFAXDL

SEQ ID NO: 418 - protein, 9 aa, synthetic construct
  MOD_RES 2: note = Met(O)
  MOD_RES 7: note = 3-(4-prime-pyridyl)-L-Ala-OH
YXAYFAXEL

SEQ ID NO: 419 - protein, 9 aa, synthetic construct
  MOD_RES 2: note = Met(O)
  MOD_RES 3: note = 3,4-difluoro-L-Phe-OH
  MOD_RES 7: note = Beta-cyclohexyl-L-Ala-OH
YXXSAAXRL